(19) United States
(12) Patent Application Publication
(10) Pub. No.: US 2016/0361360 A1
(43) Pub. Date: Dec. 15, 2016

(54) DISEASE THERAPY WITH CHIMERIC ANTIGEN RECEPTOR (CAR) CONSTRUCTS AND T CELLS (CAR-T) OR NK CELLS (CAR-NK) EXPRESSING CAR CONSTRUCTS

(71) Applicant: Immunomedics, Inc., Morris Plains, NJ (US)

(72) Inventors: Chien-Hsing Chang, Downingtown, PA (US); Donglin Liu, Kendall Park, NJ (US); David M. Goldenberg, Mendham, NJ (US)

(21) Appl. No.: 15/179,472

(22) Filed: Jun. 10, 2016

Related U.S. Application Data
(60) Provisional application No. 62/174,894, filed on Jun. 12, 2015.
(60) Provisional application No. 62/193,853, filed on Jul. 17, 2015.

(51) Int. Cl. A61K 35/17; C07K 14/705; C07K 14/725; C12N 5/0783; C07K 16/28; C07K 16/30; A61K 47/48; C07K 16/44; A61K 38/21

(52) U.S. Cl. CPC A61K 35/17; C07K 16/44; C07K 14/70521; C07K 14/70578; C07K 14/7051; C07K 14/70517; A61K 38/212; C07K 16/28; C07K 16/3007; C07K 16/2818; A61K 47/48615; A61K 47/48384; C12N 5/0636; C12N 5/0646; C07K 16/2833; C07K 16/3046; C07K 2317/24; C07K 2319/03; C07K 2317/622; C12N 2510/00; A61K 2039/507

(57) ABSTRACT

The present invention concerns CAR, CAR-T and CAR-NK constructs, preferably comprising a scFv antibody fragment against a disease-associated antigen or a hapten. More preferably, the antigen is a TAA, such as Trop-2. The constructs may be administered to a subject with a disease, such as cancer, autoimmune disease, or immune dysfunction disease, to induce an immune response against disease-associated cells. Where the constructs bind to a hapten, the subject is first treated with a hapten-conjugated antibody that binds to a disease associated antigen. Therapy may be supplemented by other treatments, such as debulking procedures (e.g., surgery, chemotherapy, radiation therapy) or coadministration of other agents. More preferably, administration of the construct is preceded by predosing with an unconjugated antibody that binds to the same disease-associated antigen. Most preferably, an antibody against CD74 or HLA-DR is administered to reduce systemic immunotoxicity induced by the constructs.

### RELATED APPLICATIONS

This application claims the benefit under 35 U.S.C. 119(e) of U.S. Provisional Patent Application 62/174,894, filed Jun. 12, 2015, and 62/193,853, filed Jul. 17, 2015, the text of each of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Jun. 7, 2016, is named IMM361US1_SL and is 39,678 bytes in size.

### BACKGROUND OF THE INVENTION

Field of the Invention

The present invention concerns chimeric antigen receptor (CAR) constructs and T cells (CAR-T) or NK cells (CAR-NK) engineered to express such CAR constructs, of use to treat a variety of disease states. The CAR constructs are designed to bind to target cells either directly, by incorporation of an antibody against an antigen expressed by the target cell, or indirectly, by incorporation of an antibody against a hapten. The hapten may be associated with the target cell using a hapten-conjugated antibody against an antigen expressed by the target cell. In certain preferred embodiments, the target cell antigen may be Trop-2 and the disease to be treated may be a Trop-2 expressing cancer. However, the person of ordinary skill will realize that antigens expressed by target cells associated with many different disease states are known, and any such antigen may be targeted by the CAR constructs disclosed herein. In preferred embodiments, the CAR may comprise a scFv or Fab antibody fragment, a CD8 hinge, the CD28 transmembrane domain, the co-stimulatory signaling domain of CD28, the co-stimulatory signaling domain of 4-1BB (CD137) and/or the cytoplasmic signaling domain of CD3ζ. In more preferred embodiments, the scFv or Fab may be derived from antibodies h679 (anti-HSG), h734 (anti-In-DTPA), hRS7 (anti-Trop-2) or hMN-15 (anti-CEACAM-6). In one embodiment, the T cells or NK cells used to generate the CAR-T or CAR-NK constructs are autologous cells obtained from the patient to be treated. More preferably, the T cells or NK cells used to generate the constructs are allogeneic cells. The CAR-T or CAR-NK therapeutic constructs are administered in vivo and induce an immune response against the disease-associated target cells. The CAR-T or CAR-NK constructs may be administered with or without a hapten-conjugated antibody, which may be used also in combination with one or more other therapeutic agents, such as a cytokine, an interferon, an antibody-drug conjugate (ADC) or a checkpoint inhibitor antibody. The combinations may be administered simultaneously or sequentially. In more preferred embodiments, the CAR-T or CAR-NK may be administered with an anti-CD74 or anti-HLA-DR antibody, to reduce the immunotoxicity induced by the construct.

### BACKGROUND

Chimeric antigen receptors (CARs, also known as chimeric T cell receptors) are synthetic constructs that are designed to be expressed in host T cells or NK cells and to induce an immune response against a specific target antigen and cells expressing that antigen. The CAR typically comprises an antibody fragment, such as a scFv or Fab fragment, incorporated in a fusion protein that also comprises additional components, such as a CD3-ζ or CD28 transmembrane domain and selective T-cell activating moieties, including the endodomains of CD3-ζ, CD28, OX40, 4-1BB, Lck and/or ICOS. Various combinations of such elements have been used.

The construction and use of CAR and CAR-T were reviewed by Sadelain et al. (Cancer Discov 3:388-98, 2013). As discussed in Sadelain et al. (2013), the design of CAR constructs has evolved through several generations. First generation CARs comprised a scFv attached to a CD3-ζ transmembrane domain, with an intracellular CD3-ζ or FcRγ endodomain. Such early constructs provided for T-cell activation only, and were generally found to be not clinically effective when tested in human subjects (Sadelain et al., 2013). Second generation CAR constructs provided a dual signaling function to combine T-cell activation with costimulatory signals, such as cytokine (e.g., IL-2, IL-7, IL-15, IL-21) release (Sadelain et al., 2013). The second generation constructs comprised CD28 or CD3-ζ transmembrane domains, attached to two or more intracellular effectors selected from CD28 endodomain, CD3-ζ endodomain, ICOS, 4-1BB, DAP10 and OX40. Third generation CARs comprised three or more signaling functions, typically incorporating CD28 transmembrane and endodomains, attached to the signaling subunits of 4-1BB, OX-40 or Lck, and the cytoplasmic domain of CD3-ζ. More recent clinical trials with second or third generation CAR-T have shown some promising results. Anti-CD19 CAR-T therapy has been reported to be effective for treatment of B-cell malignancies, with 1 complete response (CR) and 1 stable disease out of 4 CLL patients treated in a preliminary study (Kochendorfer et al., Blood 119:2709-20, 2012). Ramos et al. (Cancer J 20:112-18, 2014) reviewed published phase 1 trials of anti-CD19 CAR-T in patients with relapsed B-cell malignancies. One trial using a second generation anti-CD19 CAR-T reported that of 5 patients with refractory B-cell ALL, all 5 achieved complete remission (negative minimal residual disease) after treatment with cyclophosphamide and CAR-T infusion (Ramos et al., 2014). The potential duration of remission was not known. Ritchie et al. (Mol Ther 21:2122-29, 2013) reported a phase 1 trial of a second generation anti-Le^{Y} CAR-T given to AML patients, following preconditioning with fludarabine. One of four patients achieved a cytogenetic remission, while a second showed a protracted remission of up to 23 months.

More recently, CAR constructs have been used to direct natural killer (NK) cell activity, reviewed by Hermanson & Kaufman (2015, Front Immunol 6:195) and Carlsten & Childs (2015, Front Immunol 6:266). Like T cells, NK cells can be transfected with CAR expression constructs and used to induce an immune response. Because NK cells do not require HLA matching, they can be used as allogeneic effector cells (Harmanson & Kaufman, 2015). Also, peripheral blood NK cells (PB-NK), of use for therapy, may be isolated from donors by a simple blood draw. The CAR constructs of use may contain similar elements to those used to make CAR-T cells. CAR-NK cells may contain a targeting molecule, such as a scFV or Fab, that binds to a disease associated antigen, such as a tumor-associated antigen (TAA), or to a hapten on a targetable construct. This avoids the problem that NK cells, unlike T cells, lack antigen specificity for targeting cells to be killed. The cell-targeting scFv or Fab may be linked via a transmembrane domain to one or more intracellular signaling domains to effect lymphocyte activation. Signaling domains used with CAR-NK cells have included CD3-ζ, CD28, 4-1BB, DAP10 and OX40. NK cell lines of use have included NK-92, NKG, YT, NK-YS, HANK-1, YTS and NKL cells. Transfection with genes encoding IL-2 and/or IL-15 has been proposed to reduce dependence on the need for exogenous cytokines for in vivo persistence and cell population expansion. Clinical trials using NK cells from haploidentical donors have demonstrated long-term remissions in patients with refractory acute myelogenous leukemia (Miller et al., 2004, Blood 105:3051-57). Efficacy has also been demonstrated against breast and ovarian cancer (Geller et al., 2011, Cytotherapy 13:98-107).

Nucleotide sequences encoding the cDNA of CAR constructs are incorporated in an expression vector, such as a retroviral or lentiviral vector, for transfer into T cells or NK cells. Following infection, transfection, lipofection or alternative means of introducing the vector into the host cell (CAR-T or CAR-NK), the cells are administered to a subject to induce an immune response against antigen-expressing target cells. Binding of CARs on the surface of transduced T cells or NK cells to antigens expressed by a target cells activates the T or NK cell. Activation of T or NK cells by CARs does not require antigen processing and presentation by the HLA system.

A variety of CAR-T or CAR-NK cells have been used for therapy of disease states, primarily hematopoietic cancers or some solid tumors. Antigens targeted have included α-folate receptor (ovarian and epithelial cancers), CAIX (renal carcinoma), CD19 (B-cell malignancies, CLL, ALL), CD20 (B-cell malignancies, lymphomas), CD22 (B-cell malignancies), CD23 (CLL), CD24 (pancreatic CA), CD30 (lymphomas), CD33 (AML), CD38 (NHL), CD44v7/8 (cervical CA), CEA (colorectal CA), EGFRvIII (glioblastoma), EGP-2 (multiple malignancies), EGP-40 (colorectal CA), EphA2 (glioblastoma), Erb-B2 (breast, prostate, colon CA), FBP (ovarian CA), G_{D2} (neuroblastoma, melanoma), G_{D3} (melanoma), HER2 (pancreatic CA, ovarian CA, glioblastoma, osteosarcoma), HMW-MAA (melanoma), IL-11Rα (osteosarcoma), IL-13Rα2 (glioma, glioblastoma), KDR (tumor vasculature), κ-light chain (B-cell malignancies), Lewis Y (various carcinomas), L1 (neuroblastoma), MAGE-A1 (melanoma), mesothelin (mesothelioma), MUC1 (breast and ovarian CA), MUC16 (ovarian CA), NKG2D (myeloma, ovarian CA), NY-ESO-1 (multiple myeloma), oncofetal antigen (various tumors), PSCA (prostate CA), PSMA (prostate CA), ROR1 (B-CLL), TAG-72 (adenocarcinomas), and VEGF-R2 (tumor neovasculature). (Sadelain et al., Cancer Discov 3:388-98, 2013).

A major concern with CAR-T therapy is the danger of a “cytokine storm” associated with intense antitumor responses mediated by large numbers of activated T cells (Sadelain et al., Cancer Discov 3:388-98, 2013). Side effects can include high fever, hypotension and/or organ failure, potentially resulting in death. The cytokines produced by CAR-NK cells differ from CAR-T cells, reducing the risk of an adverse cytokine-mediated reaction. Nevertheless, a need exists for improved CAR, CAR-T and CAR-NK constructs, with better efficacy and decreased systemic toxicity, and for adjunct therapies to reduce the risk of a cytokine storm or other systemic toxicities. A need also exists to prevent or mitigate non-tumor, on-target toxicity, where normal tissues expressing the target antigen are affected by toxicity due to the CAR-T or CAR-NK therapy also including these cells, as exemplified by the induction of a severe transient inflammatory colitis in all three cancer patients with metastatic colorectal cancer who have received CEA-targeting T cells (Parkhurst et al., Mol Ther, 19: 620-6, 2010).

### SUMMARY OF THE INVENTION

The present invention provides compositions and methods for therapeutic use of novel CAR, CAR-T and CAR-NK constructs. The constructs comprise an antibody moiety, preferably a scFv or Fab, attached via a linker to a transmembrane domain and two or more intracellular signaling domains, such as CD28 endodomain, CD3-ζ endodomain, and the signaling moieties of ICOS, 4-1BB (CD137), DAP10 and/or OX40. Examples of preferred embodiments of CAR constructs are shown in FIG. 1 and FIG. 2. Other exemplary structures may include a scFv/CD28/CD3-ζ or scFv/CD28/CD137/CD3-ζ. The fusion protein will comprise a linker sequence between the antibody and the rest of the CAR, to allow for increased flexibility of binding to antigen, as well as a transmembrane domain (typically CD28) connecting the scFv or Fab and intracellular effectors. As shown in FIG. 1 and FIG. 2, the fusion protein may comprise a short linker (e.g., GGGGSGGGGSGGGGS, SEQ ID NO: 18) between the the V_{H} and V_{L} portions of the scFv, and a hinge, such as a CD8α hinge, attaching the scFc to the transmembrane domain. Intracellular effectors may comprise two or more of CD28 intracellular domains (endodomain), CD3-ζ intracellular domain (endodomain), and 4-1BB intracellular domain (FIG. 1, FIG. 2). Other intracellular effectors known in the art for CAR-T and CAR-NK constructs, as discussed elsewhere in this application, may also be used.

In various embodiments, the CAR, CAR-T and CAR-NK may be designed so that the scFv, Fab or other antibody moiety binds directly to a cell surface antigen expressed by a target cell. In alternative embodiments, the CAR, CAR-T and CAR-NK may contain a scFv that binds to a hapten attached to a target cell, allowing indirect binding of CAR, CAR-T and CAR-NK to the target cell. In the latter case, the hapten may be conjugated to a different antibody or antibody fragment, which binds to a target cell antigen. Preferred haptens include HSG (histamine-succinyl-glycine) or In-DTPA (indium-diethylenetriaminepentaacetic acid). After administration of an antibody labeled with DTPA or HSG, the labeled antibody is allowed to localize to target cells or tissues. The CAR-T or CAR-NK construct is added and binds to the HSG or In-DTPA, co-localizing with the HSG- or In-DTPA-labeled antibody and inducing an immune response against the target cell. Although any known anti-hapten antibody may be utilized, in preferred embodiments the anti-HSG antibody is h679 (see, e.g., U.S. Pat. No. 7,563,439, the Figures and Examples section incorporated herein by reference) or the anti-In-DTPA antibody is h734 (see, e.g., published PCT Application WO 99/66951 or U.S. Pat. No. 7,534,431, the Figures and Examples section of each incorporated herein by reference). HSG or In-DTPA conjugated targeting antibodies may be prepared as described in the Examples below.

In alternative embodiments, a predetermined amount of a parental, unconjugated antibody is administered at least one day, preferably 1 to 10 days, prior to adding the disease-targeting CAR-T or CAR-NK construct, or the disease-targeting antibody-hapten conjugate (followed by hapten-binding CAR-T or CAR-NK). Such a predosing protocol is designed to reduce or eliminate the off-tumor, on-target toxicity against normal tissues expressing the same antigen recognized by the disease-targeting antibody in the CAR-T, CAR-NK or antibody-hapten complex. The predose may be repeated, after a delay of up to 7 days. Preclinical studies carried out in nude mice bearing xenografts of the GW-39 human colon carcinoma have shown that the antitumor activity of IMMU-130 (ADC comprising SN-38 conjugated to anti-CEACAM5 mAb hMN-14) was little affected by administering various doses of naked hMN-14 prior to treatment with IMMU-130, indicating that predosing of the parental antibody does not diminish the subsequent targeting of agents recognizing the same antigen on tumor or other diseased cells (FIG. 3). However, such predosing can mitigate the cytotoxic effect of CAR-T, CAR-NK or hapten-mAb/CAR-T or CAR-NK binding to normal tissues. While disease-associated antigens, such as tumor-associated antigens, may be specific to diseased cells, more commonly the antigen will be expressed in some normal tissues as well as on diseased cells, although typically at a lower expression level in normal cells. Pre-dosing with unconjugated antibody against the same disease-associated antigen may saturate normal tissues with lower antigen expression levels, while still allowing a cytotoxic effect against the higher antigen levels found in diseased cells, such as tumor cells. Preferably, the unconjugated antibody is administered at a dosage of from 1 to 16 mg/kg, more preferably about 10 mg/kg, with 1 to 2 predosing injections. Where two predosing injections are given, they may be administered about 1 week apart and the CAR-T or CAR-NK construct may be administered 4-6 days after the second predose injection.

In the absence of predosing, T-cell based targeted therapies may results in systemic toxicities, such as colitis. Bos et al. (Cancer Res 68:8446-55, 2008) reported on the use of autologous T-cells transduced with CEA-targeting recombinant T-cell receptors for treatment of colon cancer. According to Bos et al. (2008), “Although CEA [CEACAM5] is overexpressed in colorectal cancers, considerable levels of this antigen are present in normal intestinal epithelia.” The authors observed that CEA-targeted immunotherapy was accompanied by intestinal autoimmune colitis, with severe weight loss that occasionally resulted in death of the subject mice. Parkhurst et al. (Mol Ther 19:620-26, 2011) observed similar toxicity of CEA-targeting T cells transduced with recombinant T cell receptors, when administered to three human patients with refractory metastatic colorectal cancer. All three patients exhibited a severe transient inflammatory colitis that represented a dose-limiting toxicity. One patient showed an objective regression of cancer metasteses to lung and liver. Katz et al. (Clin Cancer Res, Epub ahead of print, Apr. 7, 2015), reported the results of a phase I trial of anti-CEA CAR-T used to treat CEA positive liver metasteses. Hepatic artery infusion (HAI) was used in an attempt to limit extrahepatic toxicity. One of six patients remained alive with stable disease at 23 months following CAR-T therapy. Although no patients suffered a grade 3 or 4 adverse event related to CAR-T therapy, febrile AEs (adverse events) were observed in 4 patients, with one patient experiencing grade 3 fever and tachycardia, apparently related to systemic IL2 infusion. One patient receiving both IL2 and CAR-T therapy developed colitis, which led to IL2 dose reduction.

Another approach to suppressing colitis without reducing anti-tumor effects of anti-CEA CAR-T in a mouse model was reported by Blat et al. (Mol Ther 22:1018-28, 2014), who used anti-CEA-CAR-Treg cells to attempt to reduce systemic toxicity of CAR-T therapy. CEA-specific CAR Tregs were reported to significantly reduce the severity of colitis compared to control Tregs, while the CEA-specific CAR Tregs significantly reduced colorectal tumor burden.

None of the studies disclosing normal tissue toxicity of CAR-T or CAR-NK cells attempted to reduce systemic toxicity by predosing with unconjugated antibody against the same target antigen. A need exists in the art for better methods of CAR-T or CAR-NK based therapy, incorporating predosing with unconjugated antibody to reduce toxicity to normal cells that also express the disease-associated antigen.

Another alternative approach to reducing systemic toxicities of CAR-T or CAR-NK constructs is to administer an antibody that reduces or prevents the hyperactivated T-cell response that is frequently seen with CAR-T, CAR-NK or checkpoint inhibitor therapies (see, e.g., Weber et al., 2015, J Clin Oncol 33:2092-99). Such systemic immune responses may be decreased or eliminated by administering anti-CD74 or anti-HLA-DR antibodies, such as hL243 or hLL1 (milatuzumab) as described below. The person of ordinary skill will realize that the claims are not limited to the specific embodiments disclosed herein and that other known anti-CD74 or anti-HLA-DR antibodies may be utilized.

Many examples of anti-CD74 antibodies are known in the art and any such known antibody, fragment, immunoconjugate or fusion protein thereof may be utilized. In a preferred embodiment, the anti-CD74 antibody is an hLL1 antibody (also known as milatuzumab). A humanized LL1 (hLL1) anti-CD74 antibody suitable for use is disclosed in U.S. Pat. No. 7,312,318, incorporated herein by reference from Col. 35, line 1 through Col. 42, line 27 and FIG. 1 through FIG. 4. However, in alternative embodiments, other known anti-CD74 antibodies may be utilized, such as LS-B1963, LS-B2594, LS-B1859, LS-B2598, LS-05525, LS-C44929, etc. (LSBio, Seattle, Wash.); LN2 (BIOLEGEND®, San Diego, Calif.); PIN. 1, SPM523, LN3, CerCLIP.1 (ABCAM®, Cambridge, Mass.); At14/19, Bu45 (SEROTEC®, Raleigh, N.C.); 1D1 (ABNOVA®, Taipei City, Taiwan); 5-329 (EBIOSCIENCE®, San Diego, Calif.); and any other anti-CD74 antibody known in the art.

In a preferred embodiment, the anti-HLA-DR antibody is an hL243 antibody (also known as IMMU-114). A humanized L243 anti-HLA-DR antibody suitable for use is disclosed in U.S. Pat. No. 7,612,180, incorporated herein by reference from Col. 46, line 45 through Col. 60, line 50 and FIG. 1 through FIG. 6. However, in alternative embodiments, other known anti-HLA-DR antibodies may be utilized, such as 1D10 (apolizumab) (Kostelny et al., 2001, Int J Cancer 93:556-65); MS-GPC-1, MS-GPC-6, MS-GPC-8, MS-GPC-10, etc. (U.S. Pat. No. 7,521,047); Lym-1, TAL 8.1, 520B, ML11C11, SPM289, MEM-267, TAL 15.1, TAL 1B5, G-7, 4D12, Bra30, etc. (Santa Cruz Biotechnology, Inc., Santa Cruz, Calif.); TAL 16.1, TU36, C120 (ABCAM®, Cambridge, Mass.); and any other anti-HLA-DR antibody known in the art.

Although any targeting antibody that binds to a cell associated with a disease may be utilized in a CAR-T or CAR-NK construct, in preferred embodiments the antibody is an anti-tumor-associated antigen (TAA) antibody, as discussed below. In a more preferred embodiment, the antibody utilized in the CAR, CAR-T and CAR-NK is an anti-Trop-2 antibody, such as hRS7. However, many other antigens expressed in disease-associated cells are known and may be utilized. Preferably, the tumor-associated antigen is alpha-fetoprotein (AFP), α-actinin-4, A3, antigen specific for A33 antibody, ART-4, B7, Ba 733, BAGE, BrE3-antigen, CA125, CAMEL, CAP-1, carbonic anhydrase IX, CASP-8/m, CCLl9, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CTLA4, CXCR4, CXCR7, CXCL12, HIF-1α, colon-specific antigen-p (CSAp), CEA (CEACAM-5), CEACAM-6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-β, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, PD1 receptor, placental growth factor, p53, PLAGL2, prostatic acid phosphatase, PSA, PRAME, PSMA, PlGF, ILGF, ILGF-R, IL-6, IL-25, RS5, RANTES, T101, SAGE, S100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker or an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207).

Exemplary antibodies against TAAs include, but are not limited to, hA19 (anti-CD19, U.S. Pat. No. 7,109,304), hR1 (anti-IGF-1R, U.S. patent application Ser. No. 13/688,812, filed Mar. 12, 2010), hPAM4 (anti-MUC5ac, U.S. Pat. No. 7,282,567), hA20 (anti-CD20, U.S. Pat. No. 7,151,164), hIMMU31 (anti-AFP, U.S. Pat. No. 7,300,655), hLL1 (anti-CD74, U.S. Pat. No. 7,312,318), hLL2 (anti-CD22, U.S. Pat. No. 5,789,554), hMu-9 (anti-CSAp, U.S. Pat. No. 7,387,773), hL243 (anti-HLA-DR, U.S. Pat. No. 7,612,180), hMN-14 (anti-CEACAM-5, U.S. Pat. No. 6,676,924), hMN-15 (anti-CEACAM-6, U.S. Pat. No. 8,287,865), hRS7 (anti-EGP-1, U.S. Pat. No. 7,238,785), hMN-3 (anti-CEACAM-6, U.S. Pat. No. 7,541,440), hRFB4 (anti-CD22, U.S. Pat. No. 9,139,649), Ab124 and Ab125 (anti-CXCR4, U.S. Pat. No. 7,138,496), the Examples section of each cited patent or application incorporated herein by reference.

The antibodies of use can be of various isotypes, preferably human IgG1, IgG2, IgG3 or IgG4, more preferably comprising human IgG1 hinge and constant region sequences. The antibodies or fragments thereof can be chimeric human-mouse, humanized (human framework and murine hypervariable (CDR) regions), or fully human, as well as variations thereof, such as half-IgG4 antibodies (referred to as “unibodies”), as described by van der Neut Kolfschoten et al. (Science 2007; 317:1554-1557). More preferably, the antibodies or fragments thereof may be designed or selected to comprise human constant region sequences that belong to specific allotypes, which may result in reduced immunogenicity when administered to a human subject. Preferred allotypes for administration include a non-Glm1 allotype (nGlm1), such as Glm3, Glm3,1, Glm3,2 or Glm3,1,2. More preferably, the allotype is selected from the group consisting of the nGlm1, Glm3, nGlm1,2 and Km3 allotypes.

Other embodiments concern combinations of CAR-T or CAR-NK therapy with cytokine treatment, such as with interferon-α, interferon-β or interferon-λ (most preferably interferon-α). Interferons are cytokine type immunomodulators that can enhance immune system function by activating NK cells and macrophages. Interferons can also have direct effects as antipathogenic agents and act in part by inducing expression of target antigens or other effector proteins. The subject interferon may be administered as free interferon, PEGylated interferon, an interferon fusion protein or interferon conjugated to an antibody.

Another promising approach to immunotherapy concerns use of antagonistic antibodies against immune checkpoint proteins (e.g., Pardoll, Nature Reviews Cancer 12:252-64, 2012). Immune checkpoints function as endogenous inhibitory pathways for immune system function that act to maintain self-tolerance and to modulate the duration and extent of immune response to antigenic stimulation (Pardoll, 2012). However, it appears that tumor tissues and possibly certain pathogens may co-opt the checkpoint system to reduce the effectiveness of host immune response, resulting in tumor growth and/or chronic infection (see, e.g., Pardoll, Nature Reviews Cancer 12:252-64, 2012; Nirschl & Drake, Clin Cancer Res 19:4917-24, 2013). Checkpoint molecules include CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3) and several others (Pardoll, Nature Reviews Cancer 12:252-64, 2012; Nirschl & Drake, Clin Cancer Res 19:4917-24, 2013). Many such antibodies are known in the art, such as lambrolizumab (MK-3475, Merck), nivolumab (BMS-936558, Bristol-Myers Squibb), pidilizumab (CT-011, CureTech Ltd.), AMP-224 (Merck), MDX-1105 (Medarex), MEDI4736 (MedImmune), MPDL3280A (Genentech), BMS-936559 (Bristol-Myers Squibb), ipilimumab (Bristol-Myers Squibb) and tremelimumab (Pfizer). Any known checkpoint inhibitor antibody may be used in combination with CAR-T or CAR-NK therapy. Antibodies against several of the checkpoint proteins are in clinical trials and have shown unexpected efficacy against tumors that were resistant to standard treatments. Exemplary checkpoint inhibitor antibodies against CTLA4 (also known as CD152), PD1 (also known as CD279) and PD-L1 (also known as CD274), are described in more detail below and may be used in combination with CAR-T or CAR-NK to enhance the effectiveness of immune response against disease cells, tissues or pathogens.

The efficacy of immune system induction for disease therapy may be enhanced by combination with other agents that, for example, reduce tumor burden prior to administration of CAR-T or CAR-NK. Antibody-drug conjugates (ADCs) can effectively reduce tumor burden in many types of cancers, as documented by pathological complete response (pCR) in neoadjuvant therapy of TNBC. Numerous exemplary ADCs are known in the art, such as IMMU-130 (labetuzumab-SN-38), IMMU-132 (hRS7-SN-38) and milatuzumab-doxorubicin or antibody conjugates of pro-2-pyrrolinodoxorubicin (Pro2PDox), as discussed below. Other exemplary ADCs of use may include gemtuzumab ozogamicin for AML (subsequently withdrawn from the market), brentuximab vedotin for ALCL and Hodgkin lymphoma, and trastuzumab emtansine for HER2-positive metastatic breast cancer (Verma et al., N Engl J Med 367:1783-91, 2012; Bross et al., Clin Cancer Res 7:1490-96, 2001; Francisco et al., Blood 102:1458-65, 2003). Numerous other candidate ADCs are currently in clinical testing, such as inotuzumab ozogamicin (Pfizer), glembatumomab vedotin (Celldex Therapeutics), SAR3419 (Sanofi-Aventis), SAR56658 (Sanofi-Aventis), AMG-172 (Amgen), AMG-595 (Amgen), BAY-94-9343 (Bayer), BIIB015 (Biogen Idec), BT062 (Biotest), SGN-75 (Seattle Genetics), SGN-CD19A (Seattle Genetics), vorsetuzumab mafodotin (Seattle Genetics), ABT-414 (AbbVie), ASG-5ME (Agensys), ASG-22ME (Agensys), ASG-16M8F (Agensys), IMGN-529 (ImmunoGen), IMGN-853 (ImmunoGen), MDX-1203 (Medarex), MLN-0264 (Millenium), RG-7450 (Roche/Genentech), RG-7458 (Roche/Genentech), RG-7593 (Roche/Genentech), RG-7596 (Roche/Genentech), RG-7598 (Roche/Genentech), RG-7599 (Roche/Genentech), RG-7600 (Roche/Genentech), RG-7636 (Roche/Genentech), anti-PSMA ADC (Progenics), lorvotuzumab mertansine (ImmunoGen), milatuzumab-doxorubicin (Immunomedics), IMMU-130 (Immunomedics) and IMMU-132 (Immunomedics). (See, e.g., Li et al., Drug Disc Ther 7:178-84, 2013; Firer & Gellerman, J Hematol Oncol 5:70, 2012; Beck et al., Discov Med 10:329-39, 2010; Mullard, Nature Rev Drug Discovery 12:329, 2013.) Any such known ADC may be used in combination with a CAR-T or CAR-NK construct as described herein. Preferably, where an ADC is used in combination with a CAR-T or CAR-NK, the ADC is administered prior to the CAR-T or CAR-NK.

In certain embodiments, the CAR-T or CAR-NK therapy may be of use for treating cancer. It is anticipated that any type of tumor and any type of tumor antigen may be targeted. Exemplary types of cancers that may be targeted include acute lymphoblastic leukemia, acute myelogenous leukemia, biliary cancer, breast cancer, cervical cancer, chronic lymphocytic leukemia, chronic myelogenous leukemia, colorectal cancer, endometrial cancer, esophageal, gastric, head and neck cancer, Hodgkin's lymphoma, lung cancer, medullary thyroid cancer, non-Hodgkin's lymphoma, multiple myeloma, renal cancer, ovarian cancer, pancreatic cancer, glioma, melanoma, liver cancer, prostate cancer, and urinary bladder cancer. However, the skilled artisan will realize that tumor-associated antigens are known for virtually any type of cancer.

Combination therapy with immunostimulatory antibodies has been reported to enhance efficacy, for example against tumor cells. Morales-Kastresana et al. (Clin Cancer Res 19:6151-62, 2013) showed that the combination of anti-PD-L1 (10B5) antibody with anti-CD137 (1D8) and anti-OX40 (OX86) antibodies provided enhanced efficacy in a transgenic mouse model of hepatocellular carcinoma. Combination of anti-CTLA4 and anti-PD1 antibodies has also been reported to be highly efficacious (Wolchok et al., N Engl J Med 369:122-33, 2013). Combination of rituximab with anti-KIR antibody, such as lirlumab (Innate Pharma) or IPH2101 (Innate Pharma), was also more efficacious against hematopoietic tumors (Kohrt et al., 2012). The person of ordinary skill will realize that the subject combination therapy may include combinations with multiple antibodies that are immunostimulatory, anti-tumor or anti-infectious agent.

Alternative antibodies that may be used for treatment of various disease states include, but are not limited to, abciximab (anti-glycoprotein IIb/IIIa), alemtuzumab (anti-CD52), bevacizumab (anti-VEGF), cetuximab (anti-EGFR), gemtuzumab (anti-CD33), ibritumomab (anti-CD20), panitumumab (anti-EGFR), rituximab (anti-CD20), tositumomab (anti-CD20), trastuzumab (anti-ErbB2), lambrolizumab (anti-PD1 receptor), nivolumab (anti-PD1 receptor), ipilimumab (anti-CTLA4), abagovomab (anti-CA-125), adecatumumab (anti-EpCAM), atlizumab (anti-IL-6 receptor), benralizumab (anti-CD125), obinutuzumab (GA101, anti-CD20), CC49 (anti-TAG-72), AB-PG1-XG1-026 (anti-PSMA, U.S. patent application Ser. No. 11/983,372, deposited as ATCC PTA-4405 and PTA-4406), D2/B (anti-PSMA, WO 2009/130575), tocilizumab (anti-IL-6 receptor), basiliximab (anti-CD25), daclizumab (anti-CD25), efalizumab (anti-CD11a), GA101 (anti-CD20; Glycart Roche), atalizumab (anti-α4 integrin), omalizumab (anti-IgE); anti-TNF-α antibodies such as CDP571 (Ofei et al., Diabetes 45:881-85, 2011), MTNFAI, M2TNFAI, M3TNFAI, M3TNFABI, M302B, M303 (Thermo Scientific, Rockford, Ill.), infliximab (Centocor, Malvern, Pa.), certolizumab pegol (UCB, Brussels, Belgium), anti-CD40L (UCB, Brussels, Belgium), adalimumab (Abbott, Abbott Park, Ill.), belimumab (Human Genome Sciences); anti-CD38 antibodies such as MOR03087 (MorphoSys AG), MOR202 (Celgene), HuMax-CD38 (Genmab) or daratumumab (Johnson & Johnson); anti-HIV antibodies such as P4/D10 (U.S. Pat. No. 8,333,971), Ab 75, Ab 76, Ab 77 (Paulik et al., Biochem Pharmacol 58:1781-90, 1999), as well as the anti-HIV antibodies described and sold by Polymun (Vienna, Austria), also described in U.S. Pat. No. 5,831,034, U.S. Pat. No. 5,911,989, and Vcelar et al., AIDS 2007; 21(16):2161-2170 and Joos et al., Antimicrob. Agents Chemother. 2006; 50(5):1773-9.

In other embodiments, the CAR-T or CAR-NK therapy may be of use to treat subjects infected with pathogenic organisms, such as bacteria, viruses or fungi. Exemplary fungi that may be treated include Microsporum, Trichophyton, Epidermophyton, Sporothrix schenckii, Cryptococcus neoformans, Coccidioides immitis, Histoplasma capsulatum, Blastomyces dermatitidis or Candida albican. Exemplary viruses include human immunodeficiency virus (HIV), herpes virus, cytomegalovirus, rabies virus, influenza virus, human papilloma virus, hepatitis B virus, hepatitis C virus, Sendai virus, feline leukemia virus, Reo virus, polio virus, human serum parvo-like virus, simian virus 40, respiratory syncytial virus, mouse mammary tumor virus, Varicella-Zoster virus, dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus or blue tongue virus. Exemplary bacteria include Bacillus anthracis, Streptococcus agalactiae, Legionella pneumophilia, Streptococcus pyogenes, Escherichia coli, Neisseria gonorrhoeae, Neisseria meningitidis, Pneumococcus spp., Hemophilis influenzae B, Treponema pallidum, Lyme disease spirochetes, Pseudomonas aeruginosa, Mycobacterium leprae, Brucella abortus, Mycobacterium tuberculosis or a Mycoplasma. Exemplary use of ADCs against infectious agents are disclosed in Johannson et al. (AIDS 20:1911-15, 2006) and Chang et al., PLos One 7:e41235, 2012).

Known antibodies against pathogens include, but are not limited to, P4D10 (anti-HIV), CR6261 (anti-influenza), exbivirumab (anti-hepatitis B), felvizumab (anti-respiratory syncytial virus), foravirumab (anti-rabies virus), motavizumab (anti-respiratory syncytial virus), palivizumab (anti-respiratory syncytial virus), panobacumab (anti-Pseudomonas), rafivirumab (anti-rabies virus), regavirumab (anti-cytomegalovirus), sevirumab (anti-cytomegalovirus), tivirumab (anti-hepatitis B), and urtoxazumab (anti-E. coli).

The subject agents may be administered in combination with one or more other immunomodulators to enhance the immune response. Immunomodulators may include, but are not limited to, a cytokine, a chemokine, a stem cell growth factor, a lymphotoxin, an hematopoietic factor, a colony stimulating factor (CSF), erythropoietin, thrombopoietin, tumor necrosis factor-α (TNF), TNF-β, granulocyte-colony stimulating factor (G-CSF), granulocyte macrophage-colony stimulating factor (GM-CSF), interferon-α, interferon-β, interferon-γ, interferon-λ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, or lymphotoxin.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1. Schematic drawing of an exemplary CAR. Figure discloses “(GGGGS)₃” as SEQ ID NO: 18.

FIG. 2. Schematic drawing of another exemplary CAR.

FIG. 3. Lack of impact on antitumor activity of IMMU-130 (ADC comprising SN-38 and labetuzumab), after predosing with unconjugated labetuzumab (anti-CEACAM5). Unconjugated labetuzumab was added at various doses (6.25, 12.5, 25 mg/kg) 1 day prior to each administration of a fixed dose (12.5 mg/kg) of IMMU-130 in a twice weekly x 2 weeks regimen in the GW-39 lung metastasis model of human colon carcinoma in nude mice (N=10). Adapted from Govindan et al, 2015, Mol Pharmaceutics, 12: 1836-47.

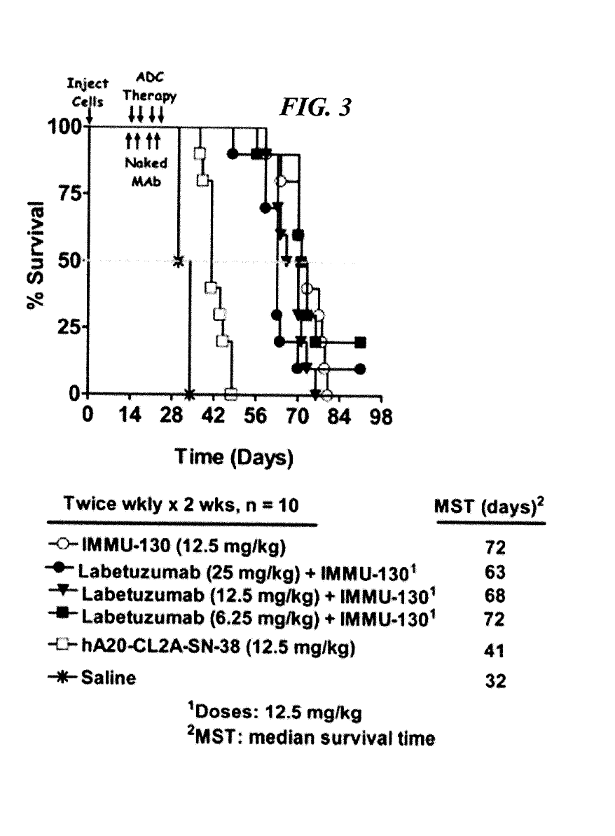

FIG. 4A. Structure of an exemplary maleimide-(PEG)ₙ-(HSG)peptide (SEQ ID NO: 23) of use for labeling antibodies with multiple HSG hapten moieties.

FIG. 4B. Structure of an exemplary SM-(PEG)ₙ moiety of use for labeling antibodies with multiple hapten moieties.

FIG. 5. Amino acid sequence of hRS7-CAR. The organization of elements within the coding sequence is shown at the top of the Figure. The complete sequence (SEQ ID NO: 26) comprises the signal peptide of CD8α (SEQ ID NO: 1), the Vk region of hRS7 (anti-Trop-2) (SEQ ID NO: 28), a linker sequence (SEQ ID NO: 18), the VH region of hRS7 (SEQ ID NO: 12), the hinge region of CD8α (SEQ ID NO: 2), the trans-membrane region of CD8α (SEQ ID NO: 3), the intracellular domain of 4-1BB (SEQ ID NO: 7), and the intracellular domain of CD3ζ (SEQ ID NO: 5). In an alternative embodiment, an optimized CD8α hinge region as disclosed in Schonfeld et al., US 20130280285 may be utilized (TTTPAPRPPTPAPTIASQPLSLRPEACRPAAGGAVHTRGLD, SEQ ID NO: 29).

FIG. 6. DNA sequence of the hRS7-CAR template (SEQ ID NO: 27).

FIG. 7. Schematic diagram of pLVX-puro-hRS7-CAR lentiviral vector.

FIG. 8. Expression of hRS7 on NK-92ML transfected with hRS7-CAR mRNA.

FIG. 9. Significant killing of Trop-2-expressing HCC1806 cells by NK-92MI transfected with hRS7-CAR mRNA.

FIG. 10. Enhanced cytotoxicity induced by NK-92MI transfected with hRS7-CAR mRNA.

FIG. 11. Expression of hRS7 on NK-92MI. Lentiviral particles were produced from lenti-X 293T cells and the supernatants were used to transduce NK-92MI. After 48-h incubation at 37° C. and 5% CO₂, cells were assessed on BD FACSCANTO flow cytometer for the expression of hRS7 by WU-AF647. The results of two experiments are shown.

FIG. 12. Histograms of Nk-92MI cells transduced with pVLX-puro-hRS7-CAR.

### DETAILED DESCRIPTION

### Definitions

Unless otherwise specified, “a” or “an” means “one or more”.

As used herein, the terms “and” and “or” may be used to mean either the conjunctive or disjunctive. That is, both terms should be understood as equivalent to “and/or” unless otherwise stated.

A “therapeutic agent” is an atom, molecule, or compound that is useful in the treatment of a disease. Examples of therapeutic agents include antibodies, antibody fragments, peptides, drugs, toxins, enzymes, nucleases, hormones, immunomodulators, antisense oligonucleotides, small interfering RNA (siRNA), chelators, boron compounds, photoactive agents, dyes, and radioisotopes.

An “antibody” as used herein refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. An “antibody” includes monoclonal, polyclonal, bispecific, multispecific, murine, chimeric, humanized and human antibodies.

A “naked antibody” is an antibody or antigen binding fragment thereof that is not attached to a therapeutic or diagnostic agent. The Fc portion of an intact naked antibody can provide effector functions, such as complement fixation and ADCC (see, e.g., Markrides, Pharmacol Rev 50:59-87, 1998). Other mechanisms by which naked antibodies induce cell death may include apoptosis. (Vaswani and Hamilton, Ann Allergy Asthma Immunol 81: 105-119, 1998.)

An “antibody fragment” is a portion of an intact antibody such as F(ab′)₂, F(ab)₂, Fab′, Fab, Fv, scFv, or dAb. Regardless of structure, an antibody fragment as used herein binds with the same antigen that is recognized by the full-length antibody. For example, antibody fragments include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). “Single-chain antibodies”, often abbreviated as “scFv” consist of a polypeptide chain that comprises both a V_{H} and a V_{L} domain which interact to form an antigen-binding site. The V_{H} and V_{L} domains are usually linked by a peptide of 1 to 25 amino acid residues. Antibody fragments also include diabodies, triabodies and single domain antibodies (dAb).

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains, including human framework region (FR) sequences. The constant domains of the antibody molecule are derived from those of a human antibody. To maintain binding activity, a limited number of FR amino acid residues from the parent (e.g., murine) antibody may be substituted for the corresponding human FR residues.

A “human antibody” is an antibody obtained from transgenic mice that have been genetically engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A human antibody also can be constructed by genetic or chromosomal transfection methods, as well as phage display technology, all of which are known in the art. (See, e.g., McCafferty et al., 1990, Nature 348:552-553 for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

As used herein, the term “antibody fusion protein” is a recombinantly produced antigen-binding molecule in which an antibody or antibody fragment is linked to another protein or peptide, such as the same or different antibody or antibody fragment or another peptide or protein. The fusion protein may comprise a single antibody component, a multivalent or multispecific combination of different antibody components or multiple copies of the same antibody component. The fusion protein may additionally comprise an antibody or an antibody fragment and a therapeutic agent.

An antibody preparation, or a composition described herein, is said to be administered in a “therapeutically effective amount” if the amount administered is physiologically significant. An agent is physiologically significant if its presence results in a detectable change in the physiology of a recipient subject. In particular embodiments, an antibody preparation is physiologically significant if its presence invokes an antitumor response or mitigates the signs and symptoms of an infectious disease state. A physiologically significant effect could also be the evocation of a humoral and/or cellular immune response in the recipient subject leading to growth inhibition or death of target cells.

CAR, CAR-T and CAR-NK Constructs

CAR constructs may be produced and used as disclosed in the following Examples. Generally, the constructs may comprise a leader sequence linked to a scFv, Fab or other antibody moiety, generally with a hinge or other linker between the scFv and a transmembrane domain. The transmembrane domain will be attached to an intracellular signaling domain, such as CD28 or CD3-ζ, and typically will include one or more co-stimulatory domains as discussed below.

The CAR, CAR-T and CAR-NK constructs of use may include any such constructs known in the art. A wide variety of CAR constructs have been reported. Ren-Heidenreich et al. (2000, Hum Gene Ther 11:9-19) disclosed a chimeric T-cell receptor comprising a scFv from the GA733.2 (anti-EGP-2) antibody, either directly fused to the transmembrane/cytoplasmic portions of FcRIγ or with a CD8α hinge between the scFv and γ chain. Activated T cells from patients were stimulated ex vivo with anti-CD3 antibody and then transduced with recombinant retrovirus encoding the chimeric receptor.

Urbanska et al. (2012, Cancer Res 72:1844-52) reported CAR constructs comprising a biotin-binding immune receptor (BBIR) incorporating avidin instead of anti-tumor antibody. After labeling tumor cells with biotinylated anti-EpCAM antibody, CAR-T cells were administered and localized to target cells by avidin-biotin binding. CAR constructs were incorporated in a lentivirus vector and in addition to the BBIR contained CD8α hinge and transmembrane sequences, attached to the intracellular domain of CD3-ζ alone, or CD3-ζ combined with the CD28 intracellular domain. Direct intratumoral injection of BBIR-CAR-T cells and biotinylated antibody in a murine xenograft model of human ovarian cancer resulted in reduced tumor growth. These constructs were further discussed in WO 2013/044225. Additional costimulatory intracellular domains of use included CD27, CD2, CD30, CD40, PD-1, LFA-1, CD7, LIGHT, NKG2C and B7-H3. Additional transmembrane domains of use could be derived from the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 or CD154.

Sadelain et al. (2013, Cancer Discov 3:388-98) discussed a large number of CAR and CAR-T constructs known in the art, comprising scFv binding moieties attached via spacer sequences to a CD3-ζ or CD28 transmembrane domain, and one or more intracellular effectors such as a CD3-ζ endodomain, CD28 endodomain, OX40, 4-1BB, Lck and/or ICOS.

Shirasu et al. (J Biomed Biotech 2012:853879) produced lentiviral CAR and CAR-T constructs incorporating a CD8 leader sequence, anti-EpCAM scFv derived from a fully human antibody, CD8 hinge, CD28 transmembrane and intracellular domains and CD3-ζ intracellular domain.

Hermanson & Kaufman (2015, Front Immunol 6:195) reported numerous CAR constructs used in CAR-NK cell lines, including anti-HER-2/mCD8α hinge/CD3ζ; anti-CD20/mCD8α hinge/CD3ζ; anti-CD19/CD8α TM/CD3ζ; anti-EpCAM/CD8α hinge/CD28/CD3ζ; anti-HLA-A2 EBNA3C/CD8α hinge/CD28/CD3ζ; anti-GD2/mCD8α hinge/CD3ζ; anti-CS1/CD28 TM/CD28/CD3ζ; anti-CD138/CD8α hinge/CD3ζ; anti-HER-2/CD8α hinge/CD137/CD3ζ; anti-PSCA/CD28 hinge/CD28 TM/CD3ζ; and anti-PSCA/DAP12 TM and signaling. The person of ordinary skill will realize that any of the known components of CAR constructs may be incorporated in T cells or NK cells to produce CAR-T or CAR-NK cells within the scope of the instant invention.

Other co-stimulatory and co-inhibitory receptors have been reported (see, e.g., Chen & Flies, Nat Rev Immunol 12:227-42, 2013), including CD28, ICOS, CTLA4, PD1, PTLA, HVEM, CD27, 4-1BB, OX40, DR3, DcR3, FAS (CD95), GITR, CD30, CD40, SLAM, CD2, 2B4, TIM1, TIM2, TIM3, TIM4, TNFR1 (CD120a), TNFR2 (CD120b), LTβR, Ly108, CD84, Ly9, CRACC, BTN1, BTN2, BTN3, TIGIT, CD226, CRTAM (CD355), CD96, CD160, LAG3, LAIR1, B7-1, RANK (CD265), TACI, BAFFR, BCMA, TWEAKR, EDAR, XEDAR, RELT, DR6, TROY, NGFR, OPG, TRAILR1-4 and B7-H1. These or other known co-factors for T-cell dependent immune response may be incorporated in the subject CAR, CAR-T and CAR-NK or alternatively may be administered as adjuvants for CAR-T or CAR-NK immunotherapy.

Although the majority of CAR, CAR-T and CAR-NK constructs have been based on the scFv antibody fragment for disease cell targeting, use of other antibody fragments has also been disclosed for this purpose. In an exemplary disclosure, Nolan et al. (1999, Clin Cancer Res 5:3928-41) disclosed use of anti-CEA Fab antibody fragments to make chimeric immunoglobulin-T cell receptors. A direct comparison showed that Fab fragments were as effective as scFv fragments for expression and antigen binding. Fab fragments may be advantageous over scFv fragments in terms of stability of antigen-binding affinity.

The CAR sequences will be incorporated in an expression vector. Various expression vectors are known in the art and any such vector may be utilized. In preferred embodiments, the vector will be a retroviral or lentiviral vector. Techniques for genetic manipulation of NK cells for cancer immunotherapy have been discussed by Carlsten & Childs (2015, Front Immunol 6:266). Viral vectors used for NK cell infection have primarily included retroviral and lentiviral vectors (Carlsten & Childs, 2015). However, decreased viability of primary NK cells undergoing retroviral transduction may limit this approach (Carlsten & Childs, 2015). Lentiviral transduction has been somewhat more effective, with efficiencies of 15 to 40% (Carlsten & Childs, 2015). Transfection by electroporation or lipofection is reported to result in lower induction of apoptosis than viral transduction, with more rapid but transient expression of the transgene(s) (Carlsten & Childs, 2015). Strategies used to increase efficacy have included transduction with IL-2 or IL-15 (promoting clone persistence and expansion), CCR7 and CXCR3 to improve migration, and various genes such as CARs, CD17, IL-2, IL-15, NKG2A and double negative TGF-β II receptor to increase cytotoxicity. The skilled artisan will realize that these and other effectors known to be of use for CAR, CAR-T and CAR-NK constructs may be utilized in the instant methods and compositions.

Interferon Therapy

In various embodiments, the CAR-T or CAR-NK constructs may be used in combination with one or more interferons, such as interferon-α, interferon-β or interferon-λ. Human interferons are well known in the art and the amino acid sequences of human interferons may be readily obtained from public databases (e.g., GenBank Accession Nos. AAA52716.1; AAA52724; AAC41702.1; EAW56871.1; EAW56870.1; EAW56869.1). Human interferons may also be commercially obtained from a variety of vendors (e.g., Cell Signaling Technology, Inc., Danvers, Mass.; Genentech, South San Francisco, Calif.; EMD Millipore, Billerica, Mass.).

Interferon-α (IFNα) has been reported to have anti-tumor activity in animal models of cancer (Ferrantini et al., 1994, J Immunol 153:4604-15) and human cancer patients (Gutterman et al., 1980, Ann Intern Med 93:399-406). IFNα can exert a variety of direct anti-tumor effects, including down-regulation of oncogenes, up-regulation of tumor suppressors, enhancement of immune recognition via increased expression of tumor surface MHC class I proteins, potentiation of apoptosis, and sensitization to chemotherapeutic agents (Gutterman et al., 1994, PNAS USA 91:1198-205; Matarrese et al., 2002, Am J Pathol 160:1507-20; Mecchia et al., 2000, Gene Ther 7:167-79; Sabaawy et al., 1999, Int J Oncol 14:1143-51; Takaoka et al, 2003, Nature 424:516-23). For some tumors, IFNα can have a direct and potent anti-proliferative effect through activation of STAT1 (Grimley et al., 1998 Blood 91:3017-27). Interferon-α2b has been conjugated to anti-tumor antibodies, such as the hL243 anti-HLA-DR antibody and depletes lymphoma and myeloma cells in vitro and in vivo (Rossi et al., 2011, Blood 118:1877-84).

Indirectly, IFNα can inhibit angiogenesis (Sidky and Borden, 1987, Cancer Res 47:5155-61) and stimulate host immune cells, which may be vital to the overall antitumor response but has been largely under-appreciated (Belardelli et al., 1996, Immunol Today 17:369-72). IFNα has a pleiotropic influence on immune responses through effects on myeloid cells (Raefsky et al, 1985, J Immunol 135:2507-12; Luft et al, 1998, J Immunol 161:1947-53), T-cells (Carrero et al, 2006, J Exp Med 203:933-40; Pilling et al., 1999, Eur J Immunol 29:1041-50), and B-cells (Le et al, 2001, Immunity 14:461-70). As an important modulator of the innate immune system, IFNα induces the rapid differentiation and activation of dendritic cells (Belardelli et al, 2004, Cancer Res 64:6827-30; Paquette et al., 1998, J Leukoc Biol 64:358-67; Santini et al., 2000, J Exp Med 191:1777-88) and enhances the cytotoxicity, migration, cytokine production and antibody-dependent cellular cytotoxicity (ADCC) of NK cells (Biron et al., 1999, Ann Rev Immunol 17:189-220; Brunda et al. 1984, Cancer Res 44:597-601).

Interferon-β has been reported to be efficacious for therapy of a variety of solid tumors. Patients treated with 6 million units of IFN-β twice a week for 36 months showed a decreased recurrence of hepatocellular carcinoma after complete resection or ablation of the primary tumor in patients with HCV-related liver cancer (Ikeda et al., 2000, Hepatology 32:228-32). Gene therapy with interferon-β induced apoptosis of glioma, melanoma and renal cell carcinoma (Yoshida et al., 2004, Cancer Sci 95:858-65). Endogenous IFN-β has been observed to inhibit tumor growth by inhibiting angiogenesis in vivo (Jablonska et al., 2010, J Clin Invest. 120:1151-64.)

IFN-λs, designated as type III interferons, are a newly described group of cytokines that consist of IFN-λ1, 2, 3 (also referred to as interleukin-29, 28A, and 28B, respectively), that are genetically encoded by three different genes located on chromosome 19 (Kotenko et al., 2003, Nat Immunol 4:69-77; Sheppard et al., 2003, Nat Immunol 4:63-8). At the protein level, IFN-λ2 and −λ3 are is highly homologous, with 96% amino acid identity, while IFN-λ1 shares approximately 81% homology with IFN-λ2 and −λ3 (Sheppard et al., 2003, Nat Immunol 4:63-8). IFN-λs activate signal transduction via the JAK/STAT pathway similar to that induced by type I IFN, including the activation of JAK1 and TYK2 kinases, the phosphorylation of STAT proteins, and the activation of the transcription complex of IFN-stimulated gene factor 3 (ISGF3) (Witte et al., 2010, Cytokine Growth Factor Rev 21:237-51; Zhou et al., 2007, J Virol 81:7749-58).

A major difference between type III and type I IFN systems is the distribution of their respective receptor complexes. IFN-α/β signals through two extensively expressed type I interferon receptors, and the resulting systemic toxicity associated with IFN-α/β administration has limited their use as therapeutic agents (Pestka et al., 2007, J Biol Chem 282:20047-51). In contrast, IFN-λs signal through a heterodimeric receptor complex consisting of unique IFN-λ receptor 1 (IFN-λR1) and IL-10 receptor 2 (IL-10R2). As previously reported (Witte et al., 2009, Genes Immun 10:702-14), IFN-λR1 has a very restricted expression pattern with the highest levels in epithelial cells, melanocytes, and hepatocytes, and the lowest level in primary central nervous system (CNS) cells. Blood immune system cells express high levels of a short IFN-λ receptor splice variant (sIFN-λR1) that inhibits IFN-λ action. The limited responsiveness of neuronal cells and immune cells implies that the severe toxicity frequently associated with IFN-α therapy may be absent or significantly reduced with IFN-λs (Witte et al., 2009, Genes Immun 10:702-14; Witte et al., 2010, Cytokine Growth Factor Rev 21:237-51). A recent publication reported that while IFN-α and IFN-λ induce expression of a common set of ISGs (interferon-stimulated genes) in hepatocytes, unlike IFN-α, administration of IFN-λ did not induce STAT activation or ISG expression in purified lymphocytes or monocytes (Dickensheets et al., 2013, J Leukoc Biol. 93, published online 12/20/12). It was suggested that IFN-λ may be superior to IFN-α for treatment of chronic HCV infection, as it is less likely to induce leukopenias that are often associated with IFN-α therapy (Dickensheets et al., 2013).

IFN-λs display structural features similar to IL-10-related cytokines, but functionally possess type I IFN-like anti-viral and anti-proliferative activity (Witte et al., 2009, Genes Immun 10:702-14; Ank et al., 2006, J Virol 80:4501-9; Robek et al., 2005, J Virol 79:3851-4). IFN-λ1 and −λ2 have been demonstrated to reduce viral replication or the cytopathic effect of various viruses, including DNA viruses (hepatitis B virus (Robek et al., 2005, J Virol 79:3851-4, Doyle et al., 2006, Hepatology 44:896-906) and herpes simplex virus 2 (Ank et al., 2008, J Immunol 180:2474-85)), ss (+) RNA viruses (EMCV; Sheppard et al., 2003, Nat Immunol 4:63-8) and hepatitis C virus (Robek et al., 2005, J Virol 79:3851-4, Doyle et al., 2006, Hepatology 44:896-906; Marcello et al., 2006, Gastroenterol 131:1887-98; Pagliaccetti et al., 2008, J Biol Chem 283:30079-89), ss (−) RNA viruses (vesicular stomatitis virus; Pagliaccetti et al., 2008, J Biol Chem 283:30079-89) and influenza-A virus (Jewell et al., 2010, J Virol 84:11515-22) and double-stranded RNA viruses, such as rotavirus (Pott et al., 2011, PNAS USA 108:7944049). IFN-λ3 has been identified from genetic studies as a key cytokine in HCV infection (Ge et al., 2009, Nature 461:399-401), and has also shown potent activity against EMCV (Dellgren et al., 2009, Genes Immun 10:125-31). A deficiency of rhinovirus-induced IFN-λ production was reported to be highly correlated with the severity of rhinovirus-induced asthma exacerbation (Contoli et al., 2006, Nature Med 12:1023-26) and IFN-λ therapy has been suggested as a new approach for treatment of allergic asthma (Edwards and Johnston, 2011, EMBO Mol Med 3:306-8; Koltsida et al., 2011, EMBO Mol Med 3:348-61).

The anti-proliferative activity of IFN-λs has been established in several human cancer cell lines, including neuroendocrine carcinoma BON1 (Zitzmann et al., 2006, Biochem Biophys Res Commun 344:1334-41), glioblastoma LN319 (Meager et al., 2005, Cytokine 31:109-18), immortalized keratinocyte HaCaT (Maher et al., 2008, Cancer Biol Ther 7:1109-15), melanoma F01 (Guenterberg et al., 2010, Mol Cancer Ther 9:510-20), and esophageal carcinoma TE-11 (Li et al., 2010, Eur J Cancer 46:180-90). In animal models, IFN-λs induce both tumor apoptosis and destruction through innate and adaptive immune responses, suggesting that local delivery of IFN-λ might be a useful adjunctive strategy in the treatment of human malignancies (Numasaki et al., 2007, J Immunol 178:5086-98). A Fab-linked interferon-λ was demonstrated to have potent anti-tumor and anti-viral activity in targeted cells (Liu et al., 2013, PLoS One 8:e63940).

In clinical settings, PEGylated IFN-λ1 (PEG-IFN-λ1) has been provisionally used for patients with chronic hepatitis C virus infection. In a phase Ib study (n=56), antiviral activity was observed at all dose levels (0.5-3.0 μg/kg), and viral load reduced 2.3 to 4.0 logs when PEG-IFN-λ1 was administrated to genotype 1 HCV patients who relapsed after IFN-α therapy (Muir et al., 2010, Hepatology 52:822-32). A phase IIb study (n=526) showed that patients with HCV genotypes 1 and 4 had significantly higher response rates to treatment with PEG-IFN-λ1 compared to PEG-IFN-α. At the same time, rates of adverse events commonly associated with type I interferon treatment were lower with PEG-IFN-λ1 than with PEG-IFN-α. Neutropenia and thrombocytopenia were infrequently observed and the rates of flu-like symptoms, anemia, and musculoskeletal symptoms decreased to about ⅓ of that seen with PEG-IFN-α treatment. However, rates of serious adverse events, depression and other common adverse events (≧10%) were similar between PEG-IFN-λ1 and PEG-IFN-α. Higher rates of hepatotoxicity were seen in the highest-dose PEG-IFN-λ1 compared with PEG-IFN-α (“Investigational Compound PEG-Interferon Lambda Achieved Higher Response Rates with Fewer Flu-like and Musculoskeletal Symptoms and Cytopenias Than PEG-Interferon Alfa in Phase IIb Study of 526 Treatment-Naive Hepatitis C Patients,” Apr. 2, 2011, Press Release from Bristol-Myers Squibb).

The therapeutic effectiveness of IFNs has been validated to date by the regulatory approval of IFN-α2 for treating hairy cell leukemia, chronic myelogenous leukemia, malignant melanoma, follicular lymphoma, condylomata acuminata, AIDs-related Kaposi sarcoma, and chronic hepatitis B and C; IFN-β for treating multiple sclerosis; and IFN-γ for treating chronic granulomatous disease and malignant osteopetrosis. When used with CAR-T or CAR-NK and/or other agents, the interferon may be administered prior to, concurrently with, or after the other agent. When administered concurrently, the interferon may be either conjugated to or separate from the other agent.

Checkpoint Inhibitor Antibodies

In certain embodiments, the CAR-T or CAR-NK constructs may be utilized in combination with one or more checkpoint inhibitors, such as checkpoint inhibitor antibodies. Studies with checkpoint inhibitor antibodies for cancer therapy have generated unprecedented response rates in cancers previously thought to be resistant to cancer treatment (see, e.g., Ott & Bhardwaj, 2013, Frontiers in Immunology 4:346; Menzies & Long, 2013, Ther Adv Med Oncol 5:278-85; Pardoll, 2012, Nature Reviews Cancer 12:252-64; Mavilio & Lugli,). Therapy with antagonistic checkpoint blocking antibodies against immune system checkpoints such as CTLA4, PD1 and PD-L1 are one of the most promising new avenues of immunotherapy for cancer and other diseases.

In contrast to the majority of anti-cancer agents, checkpoint inhibitors do not target tumor cells directly, but rather target lymphocyte receptors or their ligands in order to enhance the endogenous antitumor activity of the immune system. (Pardoll, 2012, Nature Reviews Cancer 12:252-264) Because such antibodies act primarily by regulating the immune response to diseased cells, tissues or pathogens, they may be used in combination with other therapeutic modalities, such as the subject CAR-T or CAR-NK to enhance the anti-tumor effect of such agents. Because checkpoint activation may also be associated with chronic infections (Nirschl & Drake, 2013, Clin Cancer Res 19:4917-24), such combination therapies may also be of use to treat infectious disease.

It is now clear that tumors can escape immune surveillance by co-opting certain immune-checkpoint pathways, particularly in T cells that are specific for tumor antigens (Pardoll, 2012, Nature Reviews Cancer 12:252-264). Because many such immune checkpoints are initiated by ligand-receptor interactions, they can be readily blocked by antibodies against the ligands and/or their receptors (Pardoll, 2012, Nature Reviews Cancer 12:252-264). Although checkpoint inhibitor antibodies against CTLA4, PD1 and PD-L1 are the most clinically advanced, other potential checkpoint antigens are known and may be used as the target of therapeutic antibodies, such as LAG3, B7-H3, B7-H4 and TIM3 (Pardoll, 2012, Nature Reviews Cancer 12:252-264).

Programmed cell death protein 1 (PD1, also known as CD279) encodes a cell surface membrane protein of the immunoglobulin superfamily, which is expressed in B cells and NK cells (Shinohara et al., 1995, Genomics 23:704-6; Blank et al., 2007, Cancer Immunol Immunother 56:739-45; Finger et al., 1997, Gene 197:177-87; Pardoll, 2012, Nature Reviews Cancer 12:252-264). The major role of PD1 is to limit the activity of T cells in peripheral tissues during inflammation in response to infection, as well as to limit autoimmunity (Pardoll, 2012, Nature Reviews Cancer 12:252-264). PD1 expression is induced in activated T cells and binding of PD1 to one of its endogenous ligands acts to inhibit T-cell activation by inhibiting stimulatory kinases (Pardoll, 2012, Nature Reviews Cancer 12:252-264). PD1 also acts to inhibit the TCR “stop signal” (Pardoll, 2012, Nature Reviews Cancer 12:252-264). PD1 is highly expressed on T, cells and may increase their proliferation in the presence of ligand (Pardoll, 2012, Nature Reviews Cancer 12:252-264).

Anti-PD1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Because PD1/PD-L1 and CTLA4 act by different pathways, it is possible that combination therapy with checkpoint inhibitor antibodies against each may provide an enhanced immune response.

Exemplary anti-PD1 antibodies include lambrolizumab (MK-3475, MERCK), nivolumab (BMS-936558, BRISTOL-MYERS SQUIBB), AMP-224 (MERCK), and pidilizumab (CT-011, CURETECH LTD.). Anti-PD1 antibodies are commercially available, for example from ABCAM® (AB137132), BIOLEGEND® (EH12.2H7, RMP1-14) and AFFYMETRIX EBIOSCIENCE (J105, J116, MIH4).

Programmed cell death 1 ligand 1 (PD-L1, also known as CD274 and B7-H1) is a ligand for PD1, found on activated T cells, B cells, myeloid cells and macrophages. Although there are two endogenous ligands for PD1-PD-L1 and PD-L2, anti-tumor therapies have focused on anti-PD-L1 antibodies. The complex of PD1 and PD-L1 inhibits proliferation of CD8+ T cells and reduces the immune response (Topalian et al., 2012, N Engl J Med 366:2443-54; Brahmer et al., 2012, N Eng J Med 366:2455-65). Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51).

Exemplary anti-PD-L1 antibodies include MDX-1105 (MEDAREX), MEDI4736 (MEDIMMUNE) MPDL3280A (GENENTECH) and BMS-936559 (BRISTOL-MYERS SQUIBB). Anti-PD-L1 antibodies are also commercially available, for example from AFFYMETRIX EBIOSCIENCE (MIH1).

Cytotoxic T-lymphocyte antigen 4 (CTLA4, also known as CD152) is also a member of the immunoglobulin superfamily that is expressed exclusively on T-cells. CTLA4 acts to inhibit T-cell activation and is reported to inhibit helper T-cell activity and enhance regulatory T-cell immunosuppressive activity (Pardoll, 2012, Nature Reviews Cancer 12:252-264). Although the precise mechanism of action of CTLA4 remains under investigation, it has been suggested that it inhibits T cell activation by outcompeting CD28 in binding to CD80 and CD86, as well as actively delivering inhibitor signals to the T cell (Pardoll, 2012, Nature Reviews Cancer 12:252-264). Anti-CTL4A antibodies have been used in clinical trials for treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). A significant feature of anti-CTL4A is the kinetics of anti-tumor effect, with a lag period of up to 6 months after initial treatment required for physiologic response (Pardoll, 2012, Nature Reviews Cancer 12:252-264). In some cases, tumors may actually increase in size after treatment initiation, before a reduction is seen (Pardoll, 2012, Nature Reviews Cancer 12:252-264).

Exemplary anti-CTLA4 antibodies include ipilimumab (Bristol-Myers Squibb) and tremelimumab (PFIZER). Anti-PD1 antibodies are commercially available, for example, from ABCAM® (AB134090), SINO BIOLOGICAL INC. (11159-H03H, 11159-H08H), and THERMO SCIENTIFIC PIERCE (PA5-29572, PA5-23967, PA5-26465, MA1-12205, MA1-35914). Ipilimumab has recently received FDA approval for treatment of metastatic melanoma (Wada et al., 2013, J Transl Med 11:89).

The person of ordinary skill will realize that methods of determining optimal dosages of checkpoint inhibitor antibodies to administer to a patient in need thereof, either alone or in combination with one or more other agents, may be determined by standard dose-response and toxicity studies that are well known in the art. In an exemplary embodiment, a checkpoint inhibitor antibody may preferably be administered at about 0.3-10 mg/kg, or the maximum tolerated dose, administered about every three weeks or about every six weeks. Alternatively, the checkpoint inhibitor antibody may be administered by an escalating dosage regimen including administering a first dosage at about 3 mg/kg, a second dosage at about 5 mg/kg, and a third dosage at about 9 mg/kg. Alternatively, the escalating dosage regimen includes administering a first dosage of checkpoint inhibitor antibody at about 5 mg/kg and a second dosage at about 9 mg/kg. Another stepwise escalating dosage regimen may include administering a first dosage of checkpoint inhibitor antibody about 3 mg/kg, a second dosage of about 3 mg/kg, a third dosage of about 5 mg/kg, a fourth dosage of about 5 mg/kg, and a fifth dosage of about 9 mg/kg. In another aspect, a stepwise escalating dosage regimen may include administering a first dosage of 5 mg/kg, a second dosage of 5 mg/kg, and a third dosage of 9 mg/kg. Exemplary reported dosages of checkpoint inhibitor mAbs include 3 mg/kg ipilimumab administered every three weeks for four doses; 10 mg/kg ipilimumab every three weeks for eight cycles; 10 mg/kg every three weeks for four cycles then every 12 weeks for a total of three years; 10 mg/kg MK-3475 every two or every three weeks; 2 mg/kg MK-3475 every three weeks; 15 mg/kg tremilimumab every three months; 0.1, 0.3, 1, 3 or 10 mg/kg nivolumab every two weeks for up to 96 weeks; 0.3, 1, 3, or 10 mg/kg BMS-936559 every two weeks for up to 96 weeks (Kyi & Postow, Oct. 23, 2013, FEBS Lett [Epub ahead of print]; Callahan & Wolchok, 2013, J Leukoc Biol 94:41-53).

These and other known agents that stimulate immune response to tumors and/or pathogens may be used in combination with CAR-T or CAR-NK alone or in further combination with an interferon, such as interferon-α, and/or an antibody-drug conjugate for improved cancer therapy. Other known co-stimulatory pathway modulators that may be used in combination include, but are not limited to, agatolimod, belatacept, blinatumomab, CD40 ligand, anti-B7-1 antibody, anti-B7-2 antibody, anti-B7-H4 antibody, AG4263, eritoran, anti-OX40 antibody, ISF-154, and SGN-70; B7-1, B7-2, ICAM-1, ICAM-2, ICAM-3, CD48, LFA-3, CD30 ligand, CD40 ligand, heat stable antigen, B7h, OX40 ligand, LIGHT, CD70 and CD24.

In certain embodiments, anti-KIR antibodies may also be used in combination with CAR-T or CAR-NK, interferons, ADCs and/or checkpoint inhibitor antibodies. NK cells mediate anti-tumor and anti-infectious agent activity by spontaneous cytotoxicity and by ADCC when activated by antibodies (Kohrt et al., 2014, Blood, 123: 678-86). The degree of cytotoxic response is determined by a balance of inhibitory and activating signals received by the NK cells (Kohrt et al., 2013). The killer cell immunoglobulin-like receptor (KIR) mediates an inhibitory signal that decreases NK cell response. Anti-KIR antibodies, such as lirlumab (Innate Pharma) and IPH2101 (Innate Pharma) have demonstrated anti-tumor activity in multiple myeloma (Benson et al., 2012, Blood 120:4324-33). In vitro, anti-KIR antibodies prevent the tolerogenic interaction of NK cells with target cells and augments the NK cell cytotoxic response to tumor cells (Kohrt et al., 2014, Blood, 123: 678-86). In vivo, in combination with rituximab (anti-CD20), anti-KIR antibodies at a dose of 0.5 mg/kg induced enhanced NK cell-mediated, rituximab-dependent cytotoxicity against lymphomas (Kohrt et al., 2014, Blood, 123: 678-86). Anti-KIR mAbs may be combined with ADCs, CAR-T or CAR-NK, interferons and/or checkpoint inhibitor antibodies to potentiate cytotoxicity to tumor cells or pathogenic organisms.

Antibody-Drug Conjugates

The subject CAR-T or CAR-NK constructs may be utilized in combination with one or more standard anti-cancer therapies, such as surgery, radiation therapy, chemotherapy and the like. In specific embodiments, the CAR-T or CAR-NK may be administered following use of a tumor debulking therapy, such as surgery, chemotherapy or immunotherapy. A preferred embodiment utilizes CAR-T or CAR-NK in combination with antibody-drug conjugates (ADCs).

ADCs are a potent class of therapeutic constructs that allow targeted delivery of cytotoxic agents to target cells, such as cancer cells. Because of the targeting function, these compounds show a much higher therapeutic index compared to the same systemically delivered agents. ADCs have been developed as intact antibodies or antibody fragments, such as scFvs. The antibody or fragment is linked to one or more copies of drug via a linker that is stable under physiological conditions, but that may be cleaved once inside the target cell. ADCs approved for therapeutic use include gemtuzumab ozogamicin for AML (subsequently withdrawn from the market), brentuximab vedotin for ALCL and Hodgkin lymphoma, and trastuzumab emtansine for HER2-positive metastatic breast cancer (Verma et al., 2012, N Engl J Med 367:1783-91; Bross et al., 2001, Clin Cancer Res 7:1490-96; Francisco et al., 2003, Blood 102:1458-65). Numerous other candidate ADCs are currently in clinical testing, such as inotuzumab ozogamicin (Pfizer), glembatumomab vedotin (Celldex Therapeutics), SAR3419 (Sanofi-Aventis), SAR56658 (Sanofi-Aventis), AMG-172 (Amgen), AMG-595 (Amgen), BAY-94-9343 (Bayer), BIIB015 (Biogen Idec), BT062 (Biotest), SGN-75 (Seattle Genetics), SGN-CD19A (Seattle Genetics), vorsetuzumab mafodotin (Seattle Genetics), ABT-414 (AbbVie), ASG-5ME (Agensys), ASG-22ME (Agensys), ASG-16M8F (Agensys), IMGN-529 (ImmunoGen), IMGN-853 (ImmunoGen), MDX-1203 (Medarex), MLN-0264 (Millenium), RG-7450 (Roche/Genentech), RG-7458 (Roche/Genentech), RG-7593 (Roche/Genentech), RG-7596 (Roche/Genentech), RG-7598 (Roche/Genentech), RG-7599 (Roche/Genentech), RG-7600 (Roche/Genentech), RG-7636 (Roche/Genentech), anti-PSMA ADC (Progenics), lorvotuzumab mertansine (ImmunoGen), milatuzumab-doxorubicin (Immunomedics), IMMU-130 (Immunomedics), IMMU-132 (Immunomedics) and antibody conjugates of pro-2-pyrrolinodoxorubicin. (See, e.g., Li et al., 2013, Drug Disc Ther 7:178-84; Firer & Gellerman, J Hematol Oncol 5:70; Beck et al., 2010, Discov Med 10:329-39; Mullard, 2013, Nature Rev Drug Discovery 12:329, U.S. Pat. Nos. 8,877,202; 9,095,628.) Because of the potential of ADCs to act as potent anti-cancer agents with reduced systemic toxicity, they may be used either alone or as an adjunct therapy to reduce tumor burden.

In particularly preferred embodiments, an ADC of use may be selected from the group consisting of IMMU-130 (hMN-14-SN-38), IMMU-132 (hRS7-SN-38), other antibody-SN-38 conjugates, or antibody conjugates of a prodrug form of 2-pyrrolinodoxorubicin (P2PDOX). (See, e.g., U.S. Pat. Nos. 7,999,083; 8,080,250; 8,741,300; 8,759,496; 8,999,344; 8,877,202 and 9,028,833, the Figures and Examples sections of each incorporated herein by reference.)

General Antibody Techniques

Techniques for preparing monoclonal antibodies against virtually any target antigen are well known in the art. See, for example, Kohler and Milstein, Nature 256: 495 (1975), and Coligan et al. (eds.), CURRENT PROTOCOLS IMMUNOLOGY, VOL. 1, pages 2.5.1-2.6.7 (John Wiley & Sons 1991). Briefly, monoclonal antibodies can be obtained by injecting mice with a composition comprising an antigen, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

MAbs can be isolated and purified from hybridoma cultures by a variety of well-established techniques. Such isolation techniques include affinity chromatography with Protein-A Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in METHODS IN MOLECULAR BIOLOGY, VOL. 10, pages 79-104 (The Humana Press, Inc. 1992).

After the initial raising of antibodies to the immunogen, the antibodies can be sequenced and subsequently prepared by recombinant techniques. Humanization and chimerization of murine antibodies and antibody fragments are well known to those skilled in the art. The use of antibody components derived from humanized, chimeric or human antibodies obviates potential problems associated with the immunogenicity of murine constant regions. The person of ordinary skill will realize that for human therapeutic use, antibodies that bind to human antigens, as opposed to their animal homologs, are preferred.

Chimeric Antibodies

A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. General techniques for cloning murine immunoglobulin variable domains are disclosed, for example, in Orlandi et al., Proc. Nat'l Acad. Sci. USA 86: 3833 (1989). Techniques for constructing chimeric antibodies are well known to those of skill in the art. As an example, Leung et al., Hybridoma 13:469 (1994), produced an LL2 chimera by combining DNA sequences encoding the V_{κ} and V_{H} domains of murine LL2, an anti-CD22 monoclonal antibody, with respective human κ and IgG₁ constant region domains.

Humanized Antibodies

Techniques for producing humanized MAbs are well known in the art (see, e.g., Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993)). A chimeric or murine monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. As simply transferring mouse CDRs into human FRs often results in a reduction or even loss of antibody affinity, additional modification might be required in order to restore the original affinity of the murine antibody. This can be accomplished by the replacement of one or more human residues in the FR regions with their murine counterparts to obtain an antibody that possesses good binding affinity to its epitope. See, for example, Tempest et al., Biotechnology 9:266 (1991) and Verhoeyen et al., Science 239: 1534 (1988). Generally, those human FR amino acid residues that differ from their murine counterparts and are located close to or touching one or more CDR amino acid residues would be candidates for substitution.

Human Antibodies

Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Pharmacol. 3:544-50). A fully human antibody also can be constructed by genetic or chromosomal transfection methods, as well as phage display technology, all of which are known in the art. See for example, McCafferty et al., Nature 348:552-553 (1990). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.). Recombinant Fab were cloned from the μ, γ and κ chain antibody repertoires and inserted into a phage display library (Id.). RNAs were converted to cDNAs and used to make Fab cDNA libraries using specific primers against the heavy and light chain immunoglobulin sequences (Marks et al., 1991, J. Mol. Biol. 222:581-97). Library construction was performed according to Andris-Widhopf et al. (2000, In: PHAGE DISPLAY LABORATORY MANUAL, Barbas et al. (eds), 1^{st} edition, Cold Spring Harbor Laboratory Press, Cold Spring Harbor, N.Y. pp. 9.1 to 9.22). The final Fab fragments were digested with restriction endonucleases and inserted into the bacteriophage genome to make the phage display library. Such libraries may be screened by standard phage display methods, as known in the art (see, e.g., Pasqualini and Ruoslahti, 1996, Nature 380:364-366; Pasqualini, 1999, The Quart. J. Nucl. Med. 43:159-162).

Phage display can be performed in a variety of formats, for their review, see e.g. Johnson and Chiswell, Current Opinion in Structural Biology 3:5564-571 (1993). Human antibodies may also be generated by in vitro activated B cells. See U.S. Pat. Nos. 5,567,610 and 5,229,275, incorporated herein by reference in their entirety. The skilled artisan will realize that these techniques are exemplary and any known method for making and screening human antibodies or antibody fragments may be utilized.

In another alternative, transgenic animals that have been genetically engineered to produce human antibodies may be used to generate antibodies against essentially any immunogenic target, using standard immunization protocols. Methods for obtaining human antibodies from transgenic mice are disclosed by Green et al., Nature Genet. 7:13 (1994), Lonberg et al., Nature 368:856 (1994), and Taylor et al., Int. Immun. 6:579 (1994). A non-limiting example of such a system is the XENOMOUSE® (e.g., Green et al., 1999, J. Immunol. Methods 231:11-23) from Abgenix (Fremont, Calif.). In these and similar animals, the mouse antibody genes have been inactivated and replaced by functional human antibody genes, while the remainder of the mouse immune system remains intact.

The XENOMOUSE® was transformed with germline-configured YACs (yeast artificial chromosomes) that contained portions of the human IgH and Igkappa loci, including the majority of the variable region sequences, along accessory genes and regulatory sequences. The human variable region repertoire may be used to generate antibody producing B cells, which may be processed into hybridomas by known techniques. A XENOMOUSE® immunized with a target antigen will produce human antibodies by the normal immune response, which may be harvested and/or produced by standard techniques discussed above. A variety of strains of XENOMOUSE® are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XENOMOUSE® system but may utilize any transgenic animal that has been genetically engineered to produce human antibodies.

Antibody Cloning and Production

Various techniques, such as production of chimeric or humanized antibodies, may involve procedures of antibody cloning and construction. The antigen-binding Vκ (variable light chain) and V_{H} (variable heavy chain) sequences for an antibody of interest may be obtained by a variety of molecular cloning procedures, such as RT-PCR, 5′-RACE, and cDNA library screening. The V genes of an antibody from a cell that expresses a murine antibody can be cloned by PCR amplification and sequenced. To confirm their authenticity, the cloned V_{L} and V_{H} genes can be expressed in cell culture as a chimeric Ab as described by Orlandi et al., (Proc. Natl. Acad Sci. USA, 86: 3833 (1989)). Based on the V gene sequences, a humanized antibody can then be designed and constructed as described by Leung et al. (Mol. Immunol., 32: 1413 (1995)).

cDNA can be prepared from any known hybridoma line or transfected cell line producing a murine antibody by general molecular cloning techniques (Sambrook et al., Molecular Cloning, A laboratory manual, 2^{nd} Ed (1989)). The Vκ sequence for the antibody may be amplified using the primers VK1BACK and VK1FOR (Orlandi et al., 1989) or the extended primer set described by Leung et al. (BioTechniques, 15: 286 (1993)). The V_{H} sequences can be amplified using the primer pair VH1BACK/VH1FOR (Orlandi et al., 1989) or the primers annealing to the constant region of murine IgG described by Leung et al. (Hybridoma, 13:469 (1994)). Humanized V genes can be constructed by a combination of long oligonucleotide template syntheses and PCR amplification as described by Leung et al. (Mol. Immunol., 32: 1413 (1995)).

PCR products for Vκ can be subcloned into a staging vector, such as a pBR327-based staging vector, VKpBR, that contains an Ig promoter, a signal peptide sequence and convenient restriction sites. PCR products for V_{H} can be subcloned into a similar staging vector, such as the pBluescript-based VHpBS. Expression cassettes containing the Vκ and V_{H} sequences together with the promoter and signal peptide sequences can be excised from VKpBR and VHpBS and ligated into appropriate expression vectors, such as pKh and pG1g, respectively (Leung et al., Hybridoma, 13:469 (1994)). The expression vectors can be co-transfected into an appropriate cell and supernatant fluids monitored for production of a chimeric, humanized or human antibody. Alternatively, the Vκ and V_{H} expression cassettes can be excised and subcloned into a single expression vector, such as pdHL2, as described by Gillies et al. (J. Immunol. Methods 125:191 (1989) and also shown in Losman et al., Cancer, 80:2660 (1997)).

In an alternative embodiment, expression vectors may be transfected into host cells that have been pre-adapted for transfection, growth and expression in serum-free medium. Exemplary cell lines that may be used include the Sp/EEE, Sp/ESF and Sp/ESF-X cell lines (see, e.g., U.S. Pat. Nos. 7,531,327; 7,537,930 and 7,608,425; the Examples section of each of which is incorporated herein by reference). These exemplary cell lines are based on the Sp2/0 myeloma cell line, transfected with a mutant Bcl-EEE gene, exposed to methotrexate to amplify transfected gene sequences and pre-adapted to serum-free cell line for protein expression.

Antibody Fragments

Antibody fragments which recognize specific epitopes can be generated by known techniques. Antibody fragments are antigen binding portions of an antibody, such as F(ab′)₂, Fab′, F(ab)₂, Fab, Fv, scFv and the like. F(ab′)₂ fragments can be produced by pepsin digestion of the antibody molecule and Fab′ fragments can be generated by reducing disulfide bridges of the F(ab′)₂ fragments. Alternatively, Fab′ expression libraries can be constructed (Huse et al., 1989, Science, 246:1274-1281) to allow rapid and easy identification of monoclonal Fab′ fragments with the desired specificity. F(ab)₂ fragments may be generated by papain digestion of an antibody.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are described in U.S. Pat. No. 4,704,692; U.S. Pat. No. 4,946,778; Raag and Whitlow, FASEB 9:73-80 (1995) and Bird and Walker, TIBTECH, 9: 132-137 (1991).

Techniques for producing single domain antibodies (DABs or VHH) are also known in the art, as disclosed for example in Cossins et al. (2006, Prot Express Purif 51:253-259), incorporated herein by reference. Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; Maass et al., J Immunol Methods 324:13-25, 2007). The VHH may have potent antigen-binding capacity and can interact with novel epitopes that are inaccessible to conventional VH-VL pairs. (Muyldermans et al., 2001). Alpaca serum IgG contains about 50% camelid heavy chain only IgG antibodies (HCAbs) (Maass et al., 2007). Alpacas may be immunized with known antigens, such as TNF-α, and VHHs can be isolated that bind to and neutralize the target antigen (Maass et al., 2007). PCR primers that amplify virtually all alpaca VHH coding sequences have been identified and may be used to construct alpaca VHH phage display libraries, which can be used for antibody fragment isolation by standard biopanning techniques well known in the art (Maass et al., 2007). In certain embodiments, anti-pancreatic cancer VHH antibody fragments may be utilized in the claimed compositions and methods.

An antibody fragment can be prepared by proteolytic hydrolysis of the full length antibody or by expression in E. coli or another host of the DNA coding for the fragment. An antibody fragment can be obtained by pepsin or papain digestion of full length antibodies by conventional methods. These methods are described, for example, by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

Antibody Allotypes

Immunogenicity of therapeutic antibodies is associated with increased risk of infusion reactions and decreased duration of therapeutic response (Baert et al., 2003, N Engl J Med 348:602-08). The extent to which therapeutic antibodies induce an immune response in the host may be determined in part by the allotype of the antibody (Stickler et al., 2011, Genes and immunity 12:213-21). Antibody allotype is related to amino acid sequence variations at specific locations in the constant region sequences of the antibody. The allotypes of IgG antibodies containing a heavy chain γ-type constant region are designated as Gm allotypes (1976, J Immunol 117:1056-59).

For the common IgG1 human antibodies, the most prevalent allotype is Glm1 (Stickler et al., 2011, Genes and Immunity 12:213-21). However, the Glm3 allotype also occurs frequently in Caucasians (Stickler et al., 2011). It has been reported that Glm1 antibodies contain allotypic sequences that tend to induce an immune response when administered to non-Glm1 (nGlm1) recipients, such as Glm3 patients (Stickler et al., 2011). Non-Glm1 allotype antibodies are not as immunogenic when administered to Glm1 patients (Stickler et al., 2011).

The human Glm1 allotype comprises the amino acids aspartic acid at Kabat position 356 and leucine at Kabat position 358 in the CH3 sequence of the heavy chain IgG1. The nGlm1 allotype comprises the amino acids glutamic acid at Kabat position 356 and methionine at Kabat position 358. Both Glm1 and nGlm1 allotypes comprise a glutamic acid residue at Kabat position 357 and the allotypes are sometimes referred to as DEL and EEM allotypes. A non-limiting example of the heavy chain constant region sequences for Glm1 and nGlm1 allotype antibodies is shown for the exemplary antibodies rituximab (SEQ ID NO: 19) and veltuzumab (SEQ ID NO:20).

Rituximab heavy chain variable region sequence

(SEQ ID NO: 19)

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Veltuzumab heavy chain variable region

(SEQ ID NO: 20)

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFELYSKLTVDKSRW
QQGNVFSCSVMHEALHNHYTQKSLSLSPGK

Jefferis and Lefranc (2009, mAbs 1:1-7) reviewed sequence variations characteristic of IgG allotypes and their effect on immunogenicity. They reported that the Glm3 allotype is characterized by an arginine residue at Kabat position 214, compared to a lysine residue at Kabat 214 in the Glm17 allotype. The nGlm1,2 allotype was characterized by glutamic acid at Kabat position 356, methionine at Kabat position 358 and alanine at Kabat position 431. The Glm1,2 allotype was characterized by aspartic acid at Kabat position 356, leucine at Kabat position 358 and glycine at Kabat position 431. In addition to heavy chain constant region sequence variants, Jefferis and Lefranc (2009) reported allotypic variants in the kappa light chain constant region, with the Km1 allotype characterized by valine at Kabat position 153 and leucine at Kabat position 191, the Km1,2 allotype by alanine at Kabat position 153 and leucine at Kabat position 191, and the Km3 allotype characterized by alanine at Kabat position 153 and valine at Kabat position 191.

With regard to therapeutic antibodies, veltuzumab and rituximab are, respectively, humanized and chimeric IgG1 antibodies against CD20, of use for therapy of a wide variety of hematological malignancies and/or autoimmune diseases. Table 1 compares the allotype sequences of rituximab vs. veltuzumab. As shown in Table 1, rituximab (Glm17,1) is a DEL allotype IgG1, with an additional sequence variation at Kabat position 214 (heavy chain CHI) of lysine in rituximab vs. arginine in veltuzumab. It has been reported that veltuzumab is less immunogenic in subjects than rituximab (see, e.g., Morchhauser et al., 2009, J Clin Oncol 27:3346-53; Goldenberg et al., 2009, Blood 113:1062-70; Robak & Robak, 2011, BioDrugs 25:13-25), an effect that has been attributed to the difference between humanized and chimeric antibodies. However, the difference in allotypes between the EEM and DEL allotypes likely also accounts for the lower immunogenicity of veltuzumab.

TABLE 1
Allotypes of Rituximab vs. Veltuzumab

| | | Heavy chain position and | | | | | |
|---|---|---|---|---|---|---|---|
| | | associated allotypes | | | | | |
| | | 214 | | 356/358 | | 431 | |
| | Complete allotype | (allotype) | | (allotype) | | (allotype) | |
| Rituximab | G1m17,1 | K | 17 | D/L | 1 | A | — |
| Veltuzumab | G1m3 | R | 3 | E/M | — | A | — |

In order to reduce the immunogenicity of therapeutic antibodies in individuals of nGlm1 genotype, it is desirable to select the allotype of the antibody to correspond to the Glm3 allotype, characterized by arginine at Kabat 214, and the nGlm1,2 null-allotype, characterized by glutamic acid at Kabat position 356, methionine at Kabat position 358 and alanine at Kabat position 431. Surprisingly, it was found that repeated subcutaneous administration of Glm3 antibodies over a long period of time did not result in a significant immune response. In alternative embodiments, the human IgG4 heavy chain in common with the Glm3 allotype has arginine at Kabat 214, glutamic acid at Kabat 356, methionine at Kabat 359 and alanine at Kabat 431. Since immunogenicity appears to relate at least in part to the residues at those locations, use of the human IgG4 heavy chain constant region sequence for therapeutic antibodies is also a preferred embodiment. Combinations of Glm3 IgG1 antibodies with IgG4 antibodies may also be of use for therapeutic administration.

Known Antibodies

Target Antigens and Exemplary Antibodies

In a preferred embodiment, antibodies are used that recognize and/or bind to antigens that are expressed at high levels on target cells and that are expressed predominantly or exclusively on diseased cells versus normal tissues. Exemplary antibodies of use for therapy of, for example, cancer include but are not limited to LL1 (anti-CD74), LL2 or RFB4 (anti-CD22), veltuzumab (hA20, anti-CD20), rituxumab (anti-CD20), obinutuzumab (GA101, anti-CD20), lambrolizumab (anti-PD1), nivolumab (anti-PD1), MK-3475 (anti-PD1), AMP-224 (anti-PD1), pidilizumab (anti-PD1), MDX-1105 (anti-PD-L1), MEDI4736 (anti-PD-L1), MPDL3280A (anti-PD-L1), BMS-936559 (anti-PD-L1), ipilimumab (anti-CTLA4), trevilizumab (anti-CTL4A), RS7 (anti-epithelial glycoprotein-1 (EGP-1, also known as TROP-2)), PAM4 or KC4 (both anti-mucin), MN-14 (anti-carcinoembryonic antigen (CEA, also known as CD66e or CEACAM-5), MN-15 or MN-3 (anti-CEACAM-6), Mu-9 (anti-colon-specific antigen-p), Immu 31 (an anti-alpha-fetoprotein), R1 (anti-IGF-1R), A19 (anti-CD19), TAG-72 (e.g., CC49), Tn, J591 or HuJ591 (anti-PSMA (prostate-specific membrane antigen)), AB-PG1-XG1-026 (anti-PSMA dimer), D2/B (anti-PSMA), G250 (an anti-carbonic anhydrase IX MAb), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin), BWA-3 (anti-histone H2A/H4), LG2-1 (anti-histone H3), MRA12 (anti-histone H1), PR1-1 (anti-histone H2B), LG11-2 (anti-histone H2B), LG2-2 (anti-histone H2B), and trastuzumab (anti-ErbB2). Such antibodies are known in the art (e.g., U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20050271671; 20060193865; 20060210475; 20070087001; the Examples section of each incorporated herein by reference.) Specific known antibodies of use include hPAM4 (U.S. Pat. No. 7,282,567), hA20 (U.S. Pat. No. 7,151,164), hA19 (U.S. Pat. No. 7,109,304), hIMMU-31 (U.S. Pat. No. 7,300,655), hLL1 (U.S. Pat. No. 7,312,318,), hLL2 (U.S. Pat. No. 5,789,554), hMu-9 (U.S. Pat. No. 7,387,773), hL243 (U.S. Pat. No. 7,612,180), hMN-14 (U.S. Pat. No. 6,676,924), hMN-15 (U.S. Pat. No. 8,287,865), hR1 (U.S. patent application Ser. No. 13/688,812), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575) the text of each recited patent or application is incorporated herein by reference with respect to the Figures and Examples sections.

Other useful antigens that may be targeted using the described conjugates include carbonic anhydrase IX, B7, CCL19, CCL21, CSAp, HER-2/neu, BrE3, CD1, CD1a, CD2, CD3, CD4, CD5, CD8, CD11A, CD14, CD15, CD16, CD18, CD19, CD20 (e.g., C2B8, hA20, 1F5 MAbs), CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM-5, CEACAM-6, CTLA4, alpha-fetoprotein (AFP), VEGF (e.g., AVASTIN®, fibronectin splice variant), ED-B fibronectin (e.g., L19), EGP-1 (TROP-2), EGP-2 (e.g., 17-1A), EGF receptor (ErbB1) (e.g., ERBITUX®), ErbB2, ErbB3, Factor H, FHL-1, Flt-3, folate receptor, Ga 733, GRO-β, HMGB-1, hypoxia inducible factor (HIF), HM1.24, HER-2/neu, insulin-like growth factor (ILGF), IFN-γ, IFN-α, IFN-β, IFN-λ, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, the HLA-DR antigen to which L243 binds, CD66 antigens, i.e., CD66a-d or a combination thereof, MAGE, mCRP, MCP-1, MIP-1A, MIP-1B, macrophage migration-inhibitory factor (MIF), MUC1, MUC2, MUC3, MUC4, MUC5ac, placental growth factor (PlGF), PSA (prostate-specific antigen), PSMA, PAM4 antigen, PD1 receptor, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-α, TRAIL receptor (R1 and R2), TROP-2, VEGFR, RANTES, T101, as well as cancer stem cell antigens, complement factors C3, C3a, C3b, C5a, C5, and an oncogene product.

A comprehensive analysis of suitable antigen (Cluster Designation, or CD) targets on hematopoietic malignant cells, as shown by flow cytometry and which can be a guide to selecting suitable antibodies for immunotherapy, is Craig and Foon, Blood prepublished online Jan. 15, 2008; DOL 10.1182/blood-2007-11-120535.

The CD66 antigens consist of five different glycoproteins with similar structures, CD66a-e, encoded by the carcinoembryonic antigen (CEA) gene family members, BCG, CGM6, NCA, CGM1 and CEA, respectively. These CD66 antigens (e.g., CEACAM-6) are expressed mainly in granulocytes, normal epithelial cells of the digestive tract and tumor cells of various tissues. Also included as suitable targets for cancers are cancer testis antigens, such as NY-ESO-1 (Theurillat et al., Int. J. Cancer 2007; 120(11):2411-7), as well as CD79a in myeloid leukemia (Kozlov et al., Cancer Genet. Cytogenet. 2005; 163(1):62-7) and also B-cell diseases, and CD79b for non-Hodgkin's lymphoma (Poison et al., Blood 110(2):616-623). A number of the aforementioned antigens are disclosed in U.S. Provisional Application Ser. No. 60/426,379, entitled “Use of Multi-specific, Non-covalent Complexes for Targeted Delivery of Therapeutics,” filed Nov. 15, 2002. Cancer stem cells, which are ascribed to be more therapy-resistant precursor malignant cell populations (Hill and Perris, J. Natl. Cancer Inst. 2007; 99:1435-40), have antigens that can be targeted in certain cancer types, such as CD133 in prostate cancer (Maitland et al., Ernst Schering Found. Sympos. Proc. 2006; 5:155-79), non-small-cell lung cancer (Donnenberg et al., J. Control Release 2007; 122(3):385-91), and glioblastoma (Beier et al., Cancer Res. 2007; 67(9):4010-5), and CD44 in colorectal cancer (Dalerba er al., Proc. Natl. Acad. Sci. USA 2007; 104(24)10158-63), pancreatic cancer (Li et al., Cancer Res. 2007; 67(3): 1030-7), and in head and neck squamous cell carcinoma (Prince et al., Proc. Natl. Acad. Sci. USA 2007; 104(3)973-8).

Anti-cancer antibodies have been demonstrated to bind to histones in some case. Kato et al. (1991, Hum Antibodies Hybridomas 2:94-101) reported that the lung cancer-specific human monoclonal antibody HB4C5 binds to histone H2B. Garzelli et al. (1994, Immunol Lett 39:277-82) observed that Epstein-Barr virus-transformed human B lymphocytes produce natural antibodies to histones. In certain embodiments, antibodies against histones may be of use in the subject combinations. Known anti-histone antibodies include, but are not limited to, BWA-3 (anti-histone H2A/H4), LG2-1 (anti-histone H3), MRA12 (anti-histone H1), PR1-1 (anti-histone H2B), LG11-2 (anti-histone H2B), and LG2-2 (anti-histone H2B) (see, e.g., Monestier et al., 1991, Eur J Immunol 21:1725-31; Monestier et al., 1993, Molec Immunol 30:1069-75).

For multiple myeloma therapy, suitable targeting antibodies have been described against, for example, CD38 and CD138 (Stevenson, Mol Med 2006; 12(11-12):345-346; Tassone et al., Blood 2004; 104(12):3688-96), CD74 (Stein et al., ibid.), CS1 (Tai et al., Blood 2008; 112(4):1329-37, and CD40 (Tai et al., 2005; Cancer Res. 65(13):5898-5906).

Macrophage migration inhibitory factor (MIF) is an important regulator of innate and adaptive immunity and apoptosis. It has been reported that CD74 is the endogenous receptor for MIF (Leng et al., 2003, J Exp Med 197:1467-76). The therapeutic effect of antagonistic anti-CD74 antibodies on MIF-mediated intracellular pathways may be of use for treatment of a broad range of disease states, such as cancers of the bladder, prostate, breast, lung, colon and chronic lymphocytic leukemia (e.g., Meyer-Siegler et al., 2004, BMC Cancer 12:34; Shachar & Haran, 2011, Leuk Lymphoma 52:1446-54). Milatuzumab (hLL1) is an exemplary anti-CD74 antibody of therapeutic use for treatment of MIF-mediated diseases.

An example of a most-preferred antibody/antigen pair is LL1, an anti-CD74 MAb (invariant chain, class II-specific chaperone, Ii) (see, e.g., U.S. Pat. Nos. 6,653,104; 7,312,318; the Examples section of each incorporated herein by reference). The CD74 antigen is highly expressed on B-cell lymphomas (including multiple myeloma) and leukemias, certain T-cell lymphomas, melanomas, colonic, lung, and renal cancers, glioblastomas, and certain other cancers (Ong et al., Immunology 98:296-302 (1999)). A review of the use of CD74 antibodies in cancer is contained in Stein et al., Clin Cancer Res. 2007 Sep. 15; 13(18 Pt 2):5556s-5563s, incorporated herein by reference. The diseases that are preferably treated with anti-CD74 antibodies include, but are not limited to, non-Hodgkin's lymphoma, Hodgkin's disease, melanoma, lung, renal, colonic cancers, glioblastome multiforme, histiocytomas, myeloid leukemias, and multiple myeloma.

In another preferred embodiment, the therapeutic combinations can be used against pathogens, since antibodies against pathogens are known. For example, antibodies and antibody fragments which specifically bind markers produced by or associated with infectious lesions, including viral, bacterial, fungal and parasitic infections, for example caused by pathogens such as bacteria, rickettsia, mycoplasma, protozoa, fungi, and viruses, and antigens and products associated with such microorganisms have been disclosed, inter alia, in Hansen et al., U.S. Pat. No. 3,927,193 and Goldenberg U.S. Pat. Nos. 4,331,647, 4,348,376, 4,361,544, 4,468,457, 4,444,744, 4,818,709 and 4,624,846, the Examples section of each incorporated herein by reference, and in Reichert and Dewitz (Nat Rev Drug Discovery 2006; 5:191-195). A review listing antibodies against infectious organisms (antitoxin and antiviral antibodies), as well as other targets, is contained in Casadevall, Clin Immunol 1999; 93(1):5-15, incorporated herein by reference. Commercially antibodies (e.g., KPL, Inc., Gaithersburg, Md.) are available against a wide variety of human pathogens including Staphylococcus aureaus (Cat. #011-90-05), Streptococcus agalactiae (Cat. #011-90-08), Streptococcus pyogenes (Cat. #01-90-07), Helicobacter pylori (Cat. #01-93-94), Borrelia burgdorferi (Cat. #05-97-91), Escherichia coli (Cat. #01-95-91; 01-95-96), Legionella spp. (Cat. #01-90-03), Listeria spp. (Cat. #01-90-90), Vibrio cholera (Cat. #01-90-50), Shigella spp. (Cat. #16-90-01), and Campylobacter spp. (Cat. #01-92-93).

In a preferred embodiment, the pathogens are selected from the group consisting of HIV virus, Mycobacterium tuberculosis, Streptococcus agalactiae, methicillin-resistant Staphylococcus aureus, Legionella pneumophilia, Streptococcus pyogenes, Escherichia coli, Neisseria gonorrhoeae, Neisseria meningitidis, Pneumococcus, Cryptococcus neoformans, Histoplasma capsulatum, Hemophilis influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, sindbis virus, lymphocytic choriomeningitis virus, wart virus, blue tongue virus, Sendai virus, feline leukemia virus, reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japonicum, Babesia bovis, Elmeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae, as disclosed in U.S. Pat. No. 6,440,416, the Examples section of which is incorporated herein by reference.

In various embodiments, the claimed methods and compositions may utilize any of a variety of antibodies known in the art. Antibodies of use may be commercially obtained from a number of known sources. For example, a variety of antibody secreting hybridoma lines are available from the American Type Culture Collection (ATCC, Manassas, Va.). A large number of antibodies against various disease targets, including but not limited to tumor-associated antigens, have been deposited at the ATCC and/or have published variable region sequences and are available for use in the claimed methods and compositions. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572,856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040, 6,451,310; 6,444,206, 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953, 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art (see, e.g., U.S. Pat. Nos. 7,531,327; 7,537,930; 7,608,425 and 7,785,880, the Examples section of each of which is incorporated herein by reference).

In other embodiments, the antibody complexes bind to a MHC class I, MHC class II or accessory molecule, such as CD40, CD54, CD80 or CD86. The antibody complex also may bind to a leukocyte activation cytokine, or to a cytokine mediator, such as NF-κB.

In certain embodiments, one of the two different targets may be a cancer cell receptor or cancer-associated antigen, particularly one that is selected from the group consisting of B-cell lineage antigens (CD19, CD20, CD21, CD22, CD23, etc.), VEGF, VEGFR, EGFR, carcinoembryonic antigen (CEA), placental growth factor (PlGF), tenascin, HER-2/neu, EGP-1, EGP-2, CD25, CD30, CD33, CD38, CD40, CD45, CD52, CD74, CD80, CD138, NCA66, CEACAM-1, CEACAM-5, CEACAM-6 (carcinoembryonic antigen-related cellular adhesion molecule 6), MUC1, MUC2, MUC3, MUC4, MUC16, IL-6, α-fetoprotein (AFP), A3, CA125, colon-specific antigen-p (CSAp), folate receptor, HLA-DR, human chorionic gonadotropin (HCG), Ia, EL-2, insulin-like growth factor (IGF) and IGF receptor, KS-1, Le(y), MAGE, necrosis antigens, PAM-4, prostatic acid phosphatase (PAP), Pr1, prostate specific antigen (PSA), prostate specific membrane antigen (PSMA), S100, T101, TAC, TAG72, TRAIL receptors, and carbonic anhydrase IX.

Other antibodies that may be used include antibodies against infectious disease agents, such as bacteria, viruses, mycoplasms or other pathogens. Many antibodies against such infectious agents are known in the art and any such known antibody may be used in the claimed methods and compositions. For example, antibodies against the gp120 glycoprotein antigen of human immunodeficiency virus I (HIV-1) are known, and certain of such antibodies can have an immunoprotective role in humans. See, e.g., Rossi et al., Proc. Natl. Acad. Sci. USA. 86:8055-8058, 1990. Known anti-HIV antibodies include the anti-envelope antibody described by Johansson et al. (AIDS, 2006 Oct. 3; 20(15):1911-5), as well as the anti-HIV antibodies described and sold by Polymun (Vienna, Austria), also described in U.S. Pat. No. 5,831,034, U.S. Pat. No. 5,911,989, and Vcelar et al., AIDS 2007; 21(16):2161-2170 and Joos et al., Antimicrob. Agents Chemother. 2006; 50(5):1773-9, all incorporated herein by reference.

Antibodies against malaria parasites can be directed against the sporozoite, merozoite, schizont and gametocyte stages. Monoclonal antibodies have been generated against sporozoites (cirumsporozoite antigen), and have been shown to neutralize sporozoites in vitro and in rodents (N. Yoshida et al., Science 207:71-73, 1980). Several groups have developed antibodies to T. gondii, the protozoan parasite involved in toxoplasmosis (Kasper et al., J. Immunol. 129:1694-1699, 1982; Id., 30:2407-2412, 1983). Antibodies have been developed against schistosomular surface antigens and have been found to act against schistosomulae in vivo or in vitro (Simpson et al., Parasitology, 83:163-177, 1981; Smith et al., Parasitology, 84:83-91, 1982: Gryzch et al., J. Immunol., 129:2739-2743, 1982; Zodda et al., J. Immunol. 129:2326-2328, 1982; Dissous et al., J. Immunol., 129:2232-2234, 1982)

Trypanosoma cruzi is the causative agent of Chagas' disease, and is transmitted by blood-sucking reduviid insects. An antibody has been generated that specifically inhibits the differentiation of one form of the parasite to another (epimastigote to trypomastigote stage) in vitro, and which reacts with a cell-surface glycoprotein; however, this antigen is absent from the mammalian (bloodstream) forms of the parasite (Sher et al., Nature, 300:639-640, 1982).

Anti-fungal antibodies are known in the art, such as anti-Sclerotinia antibody (U.S. Pat. No. 7,910,702); antiglucuronoxylomannan antibody (Zhong and Priofski, 1998, Clin Diag Lab Immunol 5:58-64); anti-Candida antibodies (Matthews and Burnie, 2001, Curr Opin Investig Drugs 2:472-76); and anti-glycosphingolipid antibodies (Toledo et al., 2010, BMC Microbiol 10:47).

Suitable antibodies have been developed against most of the microorganism (bacteria, viruses, protozoa, fungi, other parasites) responsible for the majority of infections in humans, and many have been used previously for in vitro diagnostic purposes. These antibodies, and newer antibodies that can be generated by conventional methods, are appropriate for use in the present invention.

Immunoconjugates

In certain embodiments, antibodies or fragments thereof may be conjugated to one or more therapeutic or diagnostic agents. The therapeutic agents do not need to be the same but can be different, e.g. a drug and a radioisotope. For example, ¹³¹I can be incorporated into a tyrosine of an antibody or fusion protein and a drug attached to an epsilon amino group of a lysine residue. Therapeutic and diagnostic agents also can be attached, for example to reduced SH groups and/or to carbohydrate side chains. Many methods for making covalent or non-covalent conjugates of therapeutic or diagnostic agents with antibodies or fusion proteins are known in the art and any such known method may be utilized.

A therapeutic or diagnostic agent can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995). Alternatively, the therapeutic or diagnostic agent can be conjugated via a carbohydrate moiety in the Fc region of the antibody. The carbohydrate group can be used to increase the loading of the same agent that is bound to a thiol group, or the carbohydrate moiety can be used to bind a different therapeutic or diagnostic agent.

Methods for conjugating peptides to antibody components via an antibody carbohydrate moiety are well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, incorporated herein in their entirety by reference. The general method involves reacting an antibody component having an oxidized carbohydrate portion with a carrier polymer that has at least one free amine function. This reaction results in an initial Schiff base (imine) linkage, which can be stabilized by reduction to a secondary amine to form the final conjugate.

The Fc region may be absent if the antibody used as the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); Hansen et al., U.S. Pat. No. 5,443,953 (1995), Leung et al., U.S. Pat. No. 6,254,868, incorporated herein by reference in their entirety. The engineered carbohydrate moiety is used to attach the therapeutic or diagnostic agent.

In some embodiments, a chelating agent may be attached to an antibody, antibody fragment or fusion protein and used to chelate a therapeutic or diagnostic agent, such as a radionuclide. Exemplary chelators include but are not limited to DTPA (such as Mx-DTPA), DOTA, TETA, NETA or NOTA. Methods of conjugation and use of chelating agents to attach metals or other ligands to proteins are well known in the art (see, e.g., U.S. Pat. No. 7,563,433, the Examples section of which is incorporated herein by reference).

In certain embodiments, radioactive metals or paramagnetic ions may be attached to proteins or peptides by reaction with a reagent having a long tail, to which may be attached a multiplicity of chelating groups for binding ions. Such a tail can be a polymer such as a polylysine, polysaccharide, or other derivatized or derivatizable chains having pendant groups to which can be bound chelating groups such as, e.g., ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose.

Chelates may be directly linked to antibodies or peptides, for example as disclosed in U.S. Pat. No. 4,824,659, incorporated herein in its entirety by reference. Particularly useful metal-chelate combinations include 2-benzyl-DTPA and its monomethyl and cyclohexyl analogs, used with diagnostic isotopes in the general energy range of 60 to 4,000 keV, such as ¹²⁵I, ¹³¹I, ¹²³I, ¹²⁴I, ⁶²Cu, ⁶⁴Cu, ¹⁸F, ¹¹¹In, ⁶⁷Ga, ⁶⁸Ga, ^{99m}Tc, ^{94m}Tc, ¹¹C, ¹³N, ¹⁵O, ⁷⁶Br, for radioimaging. The same chelates, when complexed with non-radioactive metals, such as manganese, iron and gadolinium are useful for MRI. Macrocyclic chelates such as NOTA, DOTA, and TETA are of use with a variety of metals and radiometals, most particularly with radionuclides of gallium, yttrium and copper, respectively. Such metal-chelate complexes can be made very stable by tailoring the ring size to the metal of interest. Other ring-type chelates such as macrocyclic polyethers, which are of interest for stably binding nuclides, such as ²²³Ra for RAIT are encompassed.

More recently, methods of ¹⁸F-labeling of use in PET scanning techniques have been disclosed, for example by reaction of F-18 with a metal or other atom, such as aluminum. The ¹⁸F—Al conjugate may be complexed with chelating groups, such as DOTA, NOTA or NETA that are attached directly to antibodies or used to label targetable constructs in pre-targeting methods. Such F-18 labeling techniques are disclosed in U.S. Pat. No. 7,563,433, the Examples section of which is incorporated herein by reference.

Another exemplary immunoconjugate was disclosed in Johannson et al. (2006, AIDS 20:1911-15), in which a doxorubicin-conjugated P4/D10 (anti-gp120) antibody was found to be highly efficacious in treating cells infected with HIV.

Camptothecin Conjugates

In certain preferred embodiments, the immunoconjugate may comprise a camptothecin drug, such as SN-38. Camptothecin (CPT) and its derivatives are a class of potent antitumor agents. Irinotecan (also referred to as CPT-11) and topotecan are CPT analogs that are approved cancer therapeutics (Iyer and Ratain, Cancer Chemother. Phamacol. 42: S31-S43 (1998)). CPTs act by inhibiting topoisomerase I enzyme by stabilizing topoisomerase I-DNA complex (Liu, et al. in The Camptothecins: Unfolding Their Anticancer Potential, Liehr J. G., Giovanella, B. C. and Verschraegen (eds), NY Acad Sci., NY 922:1-10 (2000)).

Preferred optimal dosing of immunoconjugates may include a dosage of between 3 mg/kg and 20 mg/kg, more preferably 4 to 18 mg/kg, more preferably 6 to 12 mg/kg, more preferably 8 to 10 mg/kg, preferably given either weekly, twice weekly or every other week. The optimal dosing schedule may include treatment cycles of two consecutive weeks of therapy followed by one, two, three or four weeks of rest, or alternating weeks of therapy and rest, or one week of therapy followed by two, three or four weeks of rest, or three weeks of therapy followed by one, two, three or four weeks of rest, or four weeks of therapy followed by one, two, three or four weeks of rest, or five weeks of therapy followed by one, two, three, four or five weeks of rest, or administration once every two weeks, once every three weeks or once a month. Treatment may be extended for any number of cycles, preferably at least 2, at least 4, at least 6, at least 8, at least 10, at least 12, at least 14, or at least 16 cycles. Exemplary dosages of use may include 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, and 18 mg/kg. Preferred dosages are 4, 6, 8, 9, 10, 12, 14, 16 or 18 mg/kg. The person of ordinary skill will realize that a variety of factors, such as age, general health, specific organ function or weight, as well as effects of prior therapy on specific organ systems (e.g., bone marrow) may be considered in selecting an optimal dosage of immunoconjugate, and that the dosage and/or frequency of administration may be increased or decreased during the course of therapy. The dosage may be repeated as needed, with evidence of tumor shrinkage observed after as few as 4 to 8 doses. The optimized dosages and schedules of administration disclosed herein show unexpected superior efficacy and reduced toxicity in human subjects, which could not have been predicted from animal model studies. Surprisingly, the superior efficacy allows treatment of tumors that were previously found to be resistant to one or more standard anti-cancer therapies, including the parental compound, CPT-11, from which SN-38 is derived in vivo.

An example of an immunoconjugate, referred to as MAb-CL2A-SN-38, is shown below. Methods of preparing CL2A-SN-38 and for making and using antibody conjugates thereof are known in the art (see, e.g., U.S. Pat. Nos. 7,999,083 and 8,080,250, the Examples sections of each incorporated herein by reference).

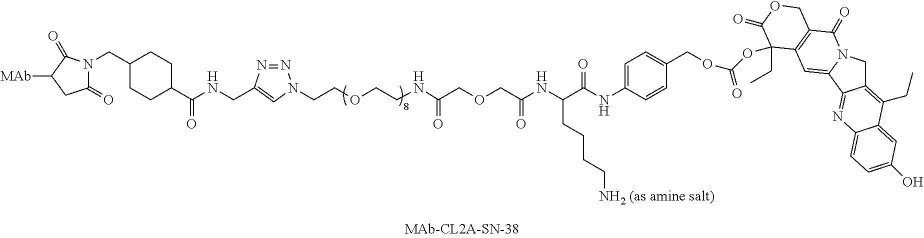

Pro-2-Pyrrolinodoxorubicin Conjugates

The compound 2-pyrrolinodoxorubicin was described first in 1996 by Schally's group, who later used it for conjugating to a number of receptor-targeted peptides for preclinical explorations (Nagy et al., 1996, Proc Natl Aad Sci USA 93:7269-73; Nagy et al., 1996, Proc Natl Acad Sci USA 96:2464-29). This is a derivative of doxorubicin, with the daunosamine nitrogen incorporated into a 5-membered enamine, making it a highly potent alkylating agent, with cytotoxicity 500-1000 times that of doxorubicin. The drug's ultratoxicity necessitates special handling in isolators, for safety. A prodrug form of the same is N-(4,4-diacetoxybutyl)doxorubicin, which is converted to 2-pyrrolinodoxorubicin in vivo. Pro-2-pyrrolinodoxorubicin (Pro-2-P-Dox) may be prepared as disclosed herein and conjugated to antibodies or antibody fragments for use in ADC therapy.

The scheme below shows the structures of Dox, 2-PDox, Pro-2-P-Dox (P2PDox), and activated Pro-2-P-Dox. For coupling to IgG, Pro-2-P-Dox may be activated with SMCC-hydrazide, a procedure that introduces acid-labile hydrazone as well as the maleimide group, the latter for conjugation to thiols of mildly reduced antibody.

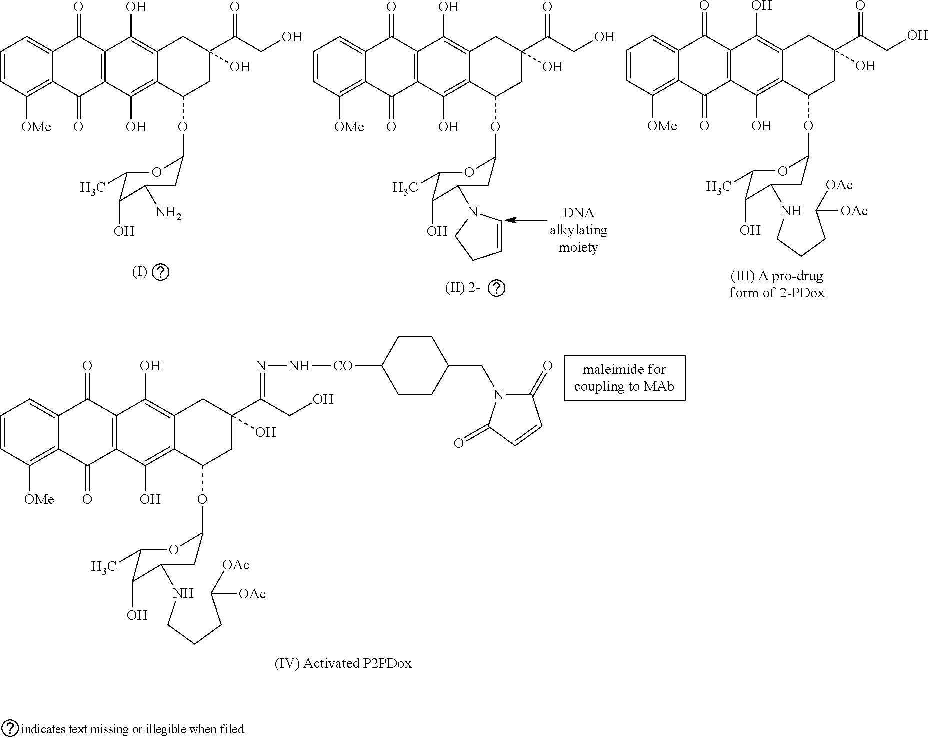

Most of the ADCs currently being clinically examined incorporate tubulin-acting, ultratoxic, maytansinoids and auristatins, which are cell-cycle-phase-specific. Anecdotally, except for trastuzumab-DM1, these ADCs appear to exhibit a relatively narrow therapeutic index clinically in solid cancers. A DNA-alkylating agent, such as 2-PDox, is cell-cycle-phase-nonspecific and should provide an improved therapeutic index. Preliminary studies (not shown) in 2 aggressive xenograft models of pancreatic and gastric cancers showed the hRS7-6 conjugate to be very active at low and safe doses (e.g., 2.25 mg/kg protein dose, or 0.064 mg/kg of drug dose), leading to complete regressions.

Reductive alkylation of doxorubicin with 4,4-diacetoxybutyraldehyde, using sodium cyanoborohydride yields P2PDox (scheme below). Diacetoxylation of commercially available 4-benzyloxybutyraldehyde, followed by hydrogenolysis and oxidation furnished the aldehyde, which was reductively coupled to doxorubicin to obtain P2PDox. The latter was activated with SMCC-hydrazide.

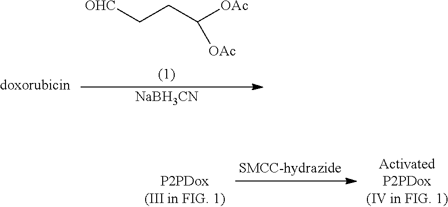 [SMILES: CC(=O)OC(CCC=O)OC(C)=O ; "NaBH3CN"]

The conjugate preparation mixed mildly reducing interchain disulfides of IgG with TCEP in PBS, followed by coupling to a 10-fold excess of activated P2PDox. The conjugates were purified on centrifuged SEC on SEPHADEX® equilibrated in 25 mM histidine, pH 7, followed by passage over a hydrophobic column. The products were formulated with trehalose and Tween 80, and lyophilized. The conjugated product, with a typical substitution of 6-7 drug/IgG, eluted as a single peak by size-exclusion HPLC, and contained typically <1% of unconjugated free drug by reversed-phase HPLC.

The person of ordinary skill will realize that P2PDox may be conjugated to any known antibody or fragment thereof, for use in ADC treatment of tumors and/or infectious disease, in combination with immunomodulating agents discussed herein.

Therapeutic Agents

In alternative embodiments, therapeutic agents such as cytotoxic agents, anti-angiogenic agents, pro-apoptotic agents, antibiotics, hormones, hormone antagonists, chemokines, drugs, prodrugs, toxins, enzymes or other agents may be used, either conjugated to the ADCs and/or other antibodies or separately administered. Drugs of use may possess a pharmaceutical property selected from the group consisting of antimitotic, antikinase, alkylating, antimetabolite, antibiotic, alkaloid, anti-angiogenic, pro-apoptotic agents and combinations thereof.

Exemplary drugs of use may include, but are not limited to, 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, Cox-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, doxorubicin, 2-pyrrolinodoxorubicine (2P-DOX), cyano-morpholino doxorubicin, doxorubicin glucuronide, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, PCI-32765, pentostatin, PSI-341, raloxifene, semustine, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839.

Toxins of use may include ricin, abrin, alpha toxin, saporin, ribonuclease (RNase), e.g., onconase, DNase I, Staphylococcal enterotoxin-A, pokeweed antiviral protein, gelonin, diphtheria toxin, Pseudomonas exotoxin, and Pseudomonas endotoxin.

Chemokines of use may include RANTES, MCAF, MIP1-alpha, MIP1-Beta and IP-10.

In certain embodiments, anti-angiogenic agents, such as angiostatin, baculostatin, canstatin, maspin, anti-VEGF antibodies, anti-PlGF peptides and antibodies, anti-vascular growth factor antibodies, anti-Flk-1 antibodies, anti-Flt-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, IP-10, Gro-β, thrombospondin, 2-methoxyoestradiol, proliferin-related protein, carboxiamidotriazole, CM101, Marimastat, pentosan polysulphate, angiopoietin-2, interferon-alpha, herbimycin A, PNU145156E, 16K prolactin fragment, Linomide (roquinimex), thalidomide, pentoxifylline, genistein, TNP-470, endostatin, paclitaxel, accutin, angiostatin, cidofovir, vincristine, bleomycin, AGM-1470, platelet factor 4 or minocycline may be of use.

Immunomodulators of use may be selected from a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β or -λ, and stem cell growth factor, such as that designated “S1 factor”. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin, tumor necrosis factor and LT.

Radionuclides of use include, but are not limited to—¹¹¹In, ¹⁷⁷Lu, ²¹²Bi, ²¹³Bi, ²¹¹At, ⁶²Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹pd, ¹⁴³Pr, ¹⁴⁹Pm, ¹⁶⁹Er, ¹⁹⁴Ir, ¹⁹⁸Au, ¹⁹⁹Au, ²¹¹Pb, and ²²⁷Th. The therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, 1-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227 and Fm-255. Decay energies of useful alpha-particle-emitting radionuclides are preferably 2,000-10,000 keV, more preferably 3,000-8,000 keV, and most preferably 4,000-7,000 keV. Additional potential radioisotopes of use include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like. Some useful diagnostic nuclides may include ¹⁸F, ⁵²Fe, ⁶²Cu, ⁶⁴Cu, ⁶⁷Cu, ⁶⁸Ga, ⁸⁶Y, ⁸⁹Zr, ⁹⁴Tc, ^{94m}Tc, ^{99m}Tc, or ¹¹¹In.

Therapeutic agents may include a photoactive agent or dye. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See Jori et al. (eds.), PHOTODYNAMIC THERAPY OF TUMORS AND OTHER DISEASES (Libreria Progetto 1985); van den Bergh, Chem. Britain (1986), 22:430. Moreover, monoclonal antibodies have been coupled with photoactivated dyes for achieving phototherapy. See Mew et al., J. Immunol. (1983), 130:1473; idem., Cancer Res. (1985), 45:4380; Oseroff et al., Proc. Natl. Acad. Sci. USA (1986), 83:8744; idem., Photochem. Photobiol. (1987), 46:83; Hasan et al., Prog. Clin. Biol. Res. (1989), 288:471; Tatsuta et al., Lasers Surg. Med. (1989), 9:422; Pelegrin et al., Cancer (1991), 67:2529.

Other useful therapeutic agents may comprise oligonucleotides, especially antisense oligonucleotides that preferably are directed against oncogenes and oncogene products, such as bcl-2 or p53. A preferred form of therapeutic oligonucleotide is siRNA. The skilled artisan will realize that any siRNA or interference RNA species may be attached to an antibody or fragment thereof for delivery to a targeted tissue. Many siRNA species against a wide variety of targets are known in the art, and any such known siRNA may be utilized in the claimed methods and compositions.

Known siRNA species of potential use include those specific for IKK-gamma (U.S. Pat. No. 7,022,828); VEGF, Flt-1 and Flk-1/KDR (U.S. Pat. No. 7,148,342); Bcl2 and EGFR (U.S. Pat. No. 7,541,453); CDC20 (U.S. Pat. No. 7,550,572); transducin (beta)-like 3 (U.S. Pat. No. 7,576,196); KRAS (U.S. Pat. No. 7,576,197); carbonic anhydrase II (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (U.S. Pat. No. 7,781,575), and apolipoprotein B (U.S. Pat. No. 7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Mirus Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools.

Methods of Therapeutic Treatment

Various embodiments concern methods of treating a cancer in a subject, such as a mammal, including humans, domestic or companion pets, such as dogs and cats, comprising administering to the subject a therapeutically effective amount of a combination of cytotoxic and/or immunomodulatory agents.

The administration of the CAR-Ts, CAR-NKs, interferons, ADCs and/or checkpoint inhibitor antibodies can be supplemented by administering concurrently or sequentially a therapeutically effective amount of another antibody that binds to or is reactive with another antigen on the surface of the target cell. Preferred additional MAbs comprise at least one humanized, chimeric or human MAb selected from the group consisting of a MAb reactive with CD4, CD5, CD8, CD14, CD15, CD16, CD19, IGF-1R, CD20, CD21, CD22, CD23, CD25, CD30, CD32b, CD33, CD37, CD38, CD40, CD40L, CD45, CD46, CD52, CD54, CD70, CD74, CD79a, CD79b, CD80, CD95, CD126, CD133, CD138, CD154, CEACAM-5, CEACAM-6, B7, AFP, PSMA, EGP-1, EGP-2, carbonic anhydrase IX, PAM4 antigen, MUC1, MUC2, MUC3, MUC4, MUC5, Ia, MIF, HM1.24, HLA-DR, tenascin, Flt-3, VEGFR, PlGF, ILGF, IL-6, IL-25, tenascin, TRAIL-R1, TRAIL-R2, complement factor C5, oncogene product, or a combination thereof. Various antibodies of use, such as anti-CD19, anti-CD20, and anti-CD22 antibodies, are known to those of skill in the art. See, for example, Ghetie et al., Cancer Res. 48:2610 (1988); Hekman et al., Cancer Immunol. Immunother. 32:364 (1991); Longo, Curr. Opin. Oncol. 8:353 (1996), U.S. Pat. Nos. 5,798,554; 6,187,287; 6,306,393; 6,676,924; 7,109,304; 7,151,164; 7,230,084; 7,230,085; 7,238,785; 7,238,786; 7,282,567; 7,300,655; 7,312,318; 7,501,498; 7,612,180; 7,670,804; and U.S. Patent Application Publ. Nos. 20080131363; 20070172920; 20060193865; and 20080138333, the Examples section of each incorporated herein by reference.

The combination therapy can be further supplemented with the administration, either concurrently or sequentially, of at least one therapeutic agent. For example, “CVB” (1.5 g/m² cyclophosphamide, 200-400 mg/m² etoposide, and 150-200 mg/m² carmustine) is a regimen used to treat non-Hodgkin's lymphoma. Patti et al., Eur. J. Haematol. 51: 18 (1993). Other suitable combination chemotherapeutic regimens are well-known to those of skill in the art. See, for example, Freedman et al., “Non-Hodgkin's Lymphomas,” in CANCER MEDICINE, VOLUME 2, 3rd Edition, Holland et al. (eds.), pages 2028-2068 (Lea & Febiger 1993). As an illustration, first generation chemotherapeutic regimens for treatment of intermediate-grade non-Hodgkin's lymphoma (NHL) include C-MOPP (cyclophosphamide, vincristine, procarbazine and prednisone) and CHOP (cyclophosphamide, doxorubicin, vincristine, and prednisone). A useful second generation chemotherapeutic regimen is m-BACOD (methotrexate, bleomycin, doxorubicin, cyclophosphamide, vincristine, dexamethasone and leucovorin), while a suitable third generation regimen is MACOP-B (methotrexate, doxorubicin, cyclophosphamide, vincristine, prednisone, bleomycin and leucovorin). Additional useful drugs include phenyl butyrate, bendamustine, and bryostatin-1.

The combinations of therapeutic agents can be formulated according to known methods to prepare pharmaceutically useful compositions, whereby the CAR-T or CAR-NK, ADC, interferon and/or checkpoint inhibitor antibody is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The subject CAR-Ts, CAR-NKs, ADCs, interferons and/or antibodies can be formulated for intravenous administration via, for example, bolus injection or continuous infusion. Preferably, the CAR-T or CAR-NK, ADC and/or antibody is infused over a period of less than about 4 hours, and more preferably, over a period of less than about 3 hours. For example, the first bolus could be infused within 30 minutes, preferably even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

Additional pharmaceutical methods may be employed to control the duration of action of the therapeutic combinations. Control release preparations can be prepared through the use of polymers to complex or adsorb the agents to be administered. For example, biocompatible polymers include matrices of poly(ethylene-co-vinyl acetate) and matrices of a polyanhydride copolymer of a stearic acid dimer and sebacic acid. Sherwood et al., Bio/Technology 10: 1446 (1992). The rate of release from such a matrix depends upon the molecular weight of the therapeutic agent, the amount of agent within the matrix, and the size of dispersed particles. Saltzman et al., Biophys. J. 55: 163 (1989); Sherwood et al., supra. Other solid dosage forms are described in Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The CAR-Ts, CAR-NKs, interferons and/or checkpoint inhibitor antibodies may be administered to a mammal subcutaneously or even by other parenteral routes, such as intravenously, intramuscularly, intraperitoneally or intravascularly. ADCs may be administered intravenously, intraperitoneally or intravascularly. Moreover, the administration may be by continuous infusion or by single or multiple boluses. Preferably, the CAR-T or CAR-NK, ADC, interferon and/or checkpoint inhibitor antibody is infused over a period of less than about 4 hours, and more preferably, over a period of less than about 3 hours.

More generally, the dosage of an administered CAR-T or CAR-NK, ADC, interferon and/or checkpoint inhibitor antibody for humans will vary depending upon such factors as the patient's age, weight, height, sex, general medical condition and previous medical history. It may be desirable to provide the recipient with a dosage of CAR-T or CAR-NK, ADC and/or antibody that is in the range of from about 1 mg/kg to 25 mg/kg as a single intravenous infusion, although a lower or higher dosage also may be administered as circumstances dictate. A dosage of 1-20 mg/kg for a 70 kg patient, for example, is 70-1,400 mg, or 41-824 mg/m² for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed in a maintenance therapy.

Alternatively, a CAR-T or CAR-NK, ADC, and/or checkpoint inhibitor antibody may be administered as one dosage every 2 or 3 weeks, repeated for a total of at least 3 dosages. Or, the combination may be administered twice per week for 4-6 weeks. If the dosage is lowered to approximately 200-300 mg/m² (340 mg per dosage for a 1.7-m patient, or 4.9 mg/kg for a 70 kg patient), it may be administered once or even twice weekly for 4 to 10 weeks. Alternatively, the dosage schedule may be decreased, namely every 2 or 3 weeks for 2-3 months. It has been determined, however, that even higher doses, such as 20 mg/kg once weekly or once every 2-3 weeks can be administered by slow i.v. infusion, for repeated dosing cycles. The dosing schedule can optionally be repeated at other intervals and dosage may be given through various parenteral routes, with appropriate adjustment of the dose and schedule.

The person of ordinary skill will realize that while the dosage schedules discussed above are relevant for ADCs, CAR-Ts, CAR-NKs and/or mAbs, the interferon agents should be administered at substantially lower dosages to avoid systemic toxicity. Dosages of interferons (such as PEGINTERFERON) for humans are more typically in the microgram range, for example 180 μg s.c. once per week, or 100 to 180 μg, or 135 μg, or 135 μg/1.73 m², or 90 μg/1.73 m², or 250 μg s.c. every other day may be of use, depending on the type of interferon.

While the CAR-Ts, CAR-NKs, interferons, ADCs and/or checkpoint inhibitor antibodies may be administered as a periodic bolus injection, in alternative embodiments the CAR-Ts, CAR-NKs, ADCs, interferons and/or checkpoint inhibitor antibodies may be administered by continuous infusion. In order to increase the Cmax and extend the PK of the therapeutic agents in the blood, a continuous infusion may be administered for example by indwelling catheter. Such devices are known in the art, such as HICKMAN®, BROVIAC® or PORT-A-CATH® catheters (see, e.g., Skolnik et al., Ther Drug Monit 32:741-48, 2010) and any such known indwelling catheter may be used. A variety of continuous infusion pumps are also known in the art and any such known infusion pump may be used. The dosage range for continuous infusion may be between 0.1 and 3.0 mg/kg per day. More preferably, the CAR-Ts, CAR-NKs, ADCs, interferons and/or checkpoint inhibitor antibodies can be administered by intravenous infusions over relatively short periods of 2 to 5 hours, more preferably 2-3 hours.

In preferred embodiments, the combination of agents is of use for therapy of cancer. Examples of cancers include, but are not limited to, carcinoma, lymphoma, glioblastoma, melanoma, sarcoma, and leukemia, myeloma, or lymphoid malignancies. More particular examples of such cancers are noted below and include: squamous cell cancer (e.g., epithelial squamous cell cancer), Ewing sarcoma, Wilms tumor, astrocytomas, lung cancer including small-cell lung cancer, non-small-cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma multiforme, cervical cancer, ovarian cancer, liver cancer, bladder cancer, hepatoma, hepatocellular carcinoma, neuroendocrine tumors, medullary thyroid cancer, differentiated thyroid carcinoma, breast cancer, ovarian cancer, colon cancer, rectal cancer, endometrial or uterine carcinoma, salivary gland carcinoma, kidney or renal cancer, prostate cancer, vulvar cancer, anal carcinoma, penile carcinoma, as well as head-and-neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). Cancers conducive to treatment methods of the present invention involve cells which express, over-express, or abnormally express IGF-1R.

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Breast Cancer, Gaucher's Disease, Gallbladder Cancer, Gastric Cancer, Gastrointestinal Carcinoid Tumor, Gastrointestinal Tumors, Germ Cell Tumors, Gestational TROPhoblastic Tumor, Hairy Cell Leukemia, Head and Neck Cancer, Hepatocellular Cancer, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Polycythemia vera, Parathyroid Cancer, Penile Cancer, Pheochromocytoma, Pituitary Tumor, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urothelial Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The methods and compositions described and claimed herein may be used to treat malignant or premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, BASIC PATHOLOGY, 2d Ed., W. B. Saunders Co., Philadelphia, pp. 68-79 (1976)).

Dysplasia is frequently a forerunner of cancer, and is found mainly in the epithelia. It is the most disorderly form of non-neoplastic cell growth, involving a loss in individual cell uniformity and in the architectural orientation of cells. Dysplasia characteristically occurs where there exists chronic irritation or inflammation. Dysplastic disorders which can be treated include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated include, but are not limited to, benign dysproliferative disorders (e.g., benign tumors, fibrocystic conditions, tissue hypertrophy, intestinal polyps or adenomas, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, meningioma, melanoma, neuroblastoma, and retinoblastoma.

Kits

Various embodiments may concern kits containing components suitable for treating or diagnosing diseased tissue in a patient. Exemplary kits may contain one or more CAR-Ts or CAR-NKs, ADCs, interferons, and/or checkpoint inhibitor antibodies as described herein. If the composition containing components for administration is not formulated for delivery via the alimentary canal, such as by oral delivery, a device capable of delivering the kit components through some other route may be included. One type of device, for applications such as parenteral delivery, is a syringe that is used to inject the composition into the body of a subject. Inhalation devices may also be used. In certain embodiments, a therapeutic agent may be provided in the form of a prefilled syringe or autoinjection pen containing a sterile, liquid formulation or lyophilized preparation.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

### Examples

The following examples are provided to illustrate, but not to limit, the claims of the present invention.

### Example 1

### Amino Acid Sequences for Chimeric Antigen Receptor Production

The design, composition, and use of several families of novel T or NK cells, each engineered with a chimeric antigen receptor (CAR) capable of binding to histamine-succinyl-glycine (HSG), DTPA-labeled indium (DTPA-In), or Trop-2, are described below. One preferred embodiment of such CAR-T or CAR-NK cells relates to third generation CARs (Sadelain et al., 2013, Cancer Discov 3:388-98) comprising, for example, an extracellularly located single-chain Fv (scFv) linked to intracellularly located signaling domains of CD28, 4-1BB (CD137) and CD3ζ via a spacer derived from the CD8α hinge and a transmembrane domain derived from CD28. Another preferred embodiment concerns second generation CARs (Sadelain et al., 2013) comprising, for example, an extracellularly located scFv linked to intracellularly located signaling domains of CD28 and CD3ζ via a spacer derived from the CD8α hinge and a transmembrane domain derived from CD28. Suitable scFvs for such CAR-T or CAR-NK cells of either the second or third generation may be obtained from h679 (anti-HSG), h734 (anti-In-DTPA), hRS7 (anti-Trop-2), hMN-15 (anti-CEACAM6), hMN-3 (anti-CEACAM6), hMN-14 (anti-CEACAM5), hR1 (anti-IGF-1R), hPAM4 (anti-mucin), KC4 (anti-mucin), hA20 (anti-CD20), hA19 (anti-CD19), hIMMU31 (anti-AFP), hLL1 (anti-CD74), hLL2 (anti-CD22), RFB4 (anti-CD22), hMu-9 (anti-CSAp), and hL243 (anti-HLA-DR).

Amino acid sequences of use are provided below. Additional sequences of use are known in the art, as disclosed in paragraphs [018] and [0105] above.

Leader peptide

(SEQ ID NO: 1)

Met Ala Leu Pro Val Thr Ala Leu Leu Leu Pro Leu
Ala Leu Leu Leu His Ala Ala Arg Pro
CD8α Hinge

(SEQ ID NO: 2)

Thr Thr Thr Pro Ala Pro Arg Pro Pro Thr Pro Ala
Pro Thr Ile Ala Ser Gln Pro Leu Ser Leu Arg Pro
Glu Ala Cys Arg Pro Ala Ala Gly Gly Ala Val His
Thr Arg Gly Leu Asp Phe Ala Cys Asp
CD8 TM

(SEQ ID NO: 3)

Ile Tyr Ile Trp Ala Pro Leu Ala Gly Thr Cys Gly
Val Leu Leu Leu Ser Leu Val Ile Thr Leu Tyr Cys
CD28 TM

(SEQ ID NO: 4)

Phe Trp Val Leu Val Val Val Gly Gly Val Leu Ala
Cys Tyr Ser Leu Leu Val Thr Val Ala Phe Ile Ile
Phe Trp Val
CD3ζICD

(SEQ ID NO: 5)

Arg Val Lys Phe Ser Arg Ser Ala Asp Ala Pro Ala
Tyr Gln Gln Gly Gln Asn Gln Leu Tyr Asn Glu Leu
Asn Leu Gly Arg Arg Glu Glu Tyr Asp Val Leu Asp
Lys Arg Arg Gly Arg Asp Pro Glu Met Gly Gly Lys
Pro Arg Arg Lys Asn Pro Gln Glu Gly Leu Tyr Asn
Glu Leu Gln Lys Asp Lys Met Ala Glu Ala Tyr Ser
Glu Ile Gly Met Lys Gly Glu Arg Arg Arg Gly Lys
Gly His Asp Gly Leu Tyr Gln Gly Leu Ser Thr Ala
Thr Lys Asp Thr Tyr Asp Ala Leu His Met Gln Ala
Leu Pro Pro Arg
CD28 ICD

(SEQ ID NO. 6)

Arg Ser Lys Arg Ser Arg Leu Leu His Ser Asp Tyr
Met Asn Met Thr Pro Arg Arg Pro Gly Pro Thr Arg
Lys His Tyr Gln Pro Tyr Ala Pro Pro Arg Asp Phe
Ala Ala Tyr Arg Ser Ile Asp
4-1BB ICD

(SEQ ID NO: 7)

Lys Arg Gly Arg Lys Lys Leu Leu Tyr Ile Phe Lys
Gln Pro Phe Met Arg Pro Val Gln Thr Thr Gln Glu
Glu Asp Gly Cys Ser Cys Arg Phe Pro Glu Glu Glu
Glu Gly Gly Cys Glu Leu
h734-V_{H}

(SEQ ID NO: 8)

QVQLQESGGGLVQPGGSLRLSCAASGFTFSHYTMSWVRQAPGKGLEWVTY
ITNGGVSSYHPDTVKGRFTVSRDNSKNTLYLQMNSLRAEDTAVYFCTRHA
VYAFAYWGQGSLVTVSS
h734-V_{L}

(SEQ ID NO: 9)

DIQLVVTQEPSFSVSPGGTVTFTCRSSAGAVTTSNYANWVQEKPGQAPRG
LIGGTTNRAPGVPARFSGSILGNKAALTITGAQADDESIYFCVLWYSDRW
VFGGGTKLKIKR
h679-V_{H}

(SEQ ID NO: 10)

QVQLQESGGDLVKPGGSLKLSCAASGFTFSIYTMSWLRQTPGKGLEWVAT
LSGDGDDIYYPDSVKGRFTISRDNAKNSLYLQMNSLRAEDTALYYCARVR
LGDWDFDVWGQGTTVTVSS
h679-V_{L}

(SEQ ID NO. 11)

DIQLTQSPSSLAVSPGERVTLTCKSSQSLFNSRTRKNYLGWYQQKPGQSP
KWYWASTRESGVPDRFSGSGSGTDFTLTINSLQAEDVAVYYCTQVYYLCT
FGAGTKLEIKR
hRS7-V_{H}

(SEQ ID NO: 12)

QVQLQQSGSELKKPGASVKVSCKASGYTFTNYGMNWVKQAPGQGLKWMGW
INTYTGEPTYTDDFKGRFAFSLDTSVSTAYLQISSLKADDTAVYFCARGG
FGSSYWYFDVWGQGSLVTVSS
hRS7-V_{L}

(SEQ ID NO: 13)

DIQLTQSPSSLSASVGDRVSITCKASQDVSIAVAWYQQKPGKAPKLLIYS
ASYRYTGVPDRFSGSGSGTDFTLTISSLQPEDFAVYYCQQHYITPLTFGA
GTKV
hMN-15-V_{H}

(SEQ ID NO: 14)

QVQLQESGGGVVQPGRSLRLSCSSSGFALTDYYMSWVRQAPGKGLEWLGF
IANKANGHTTDYSPSVKGRFTISRDNSKNTLFLQMDSLRPEDTGVYFCAR
DMGIRWNFDVWGQGTPVTVSS
hMN-15-V_{L}

(SEQ ID NO: 15)

DIQLTQSPSSLSASVGDRVTMTCSASSRVSYIHWYQQKPGKAPKRWIYGT
STLASGVPARFSGSGSGTDFTFTISSLQPEDIATYYCQQWSYNPPTFGQG
TKVEIKR
hMN-14-V_{H}

(SEQ ID NO: 16)

EVQLVESGGGVVQPGRSLRLSCSASGFDFTTYWMSWVRQAPGKGLEWIGE
IHPDSSTINYAPSLKDRFTISRDNAKNTLFLQMDSLRPEDTGVYFCASLY
FGFPWFAYWGQGTPVTVSS
hMN-14-V_{L}

(SEQ ID NO: 17)

DIQLTQSPSSLSASVGDRVTITCKASQDVGTSVAWYQQKPGKAPKLLIYW
TSTRHTGVPSRFSGSGSGTDFTFTISSLQPEDIATYYCQQYSLYRSFGQG
TKVEIKR

### Example 2

### Construction of Lentiviral Vectors for Expressing a Third-Generation CAR

HSG-Binding CAR

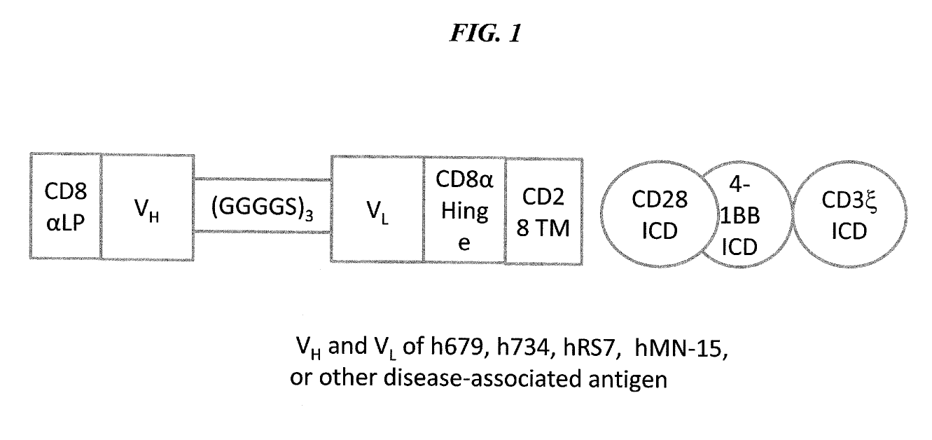

A schematic diagram showing an exemplary third-generation CAR construct is provided in FIG. 1. The CAR construct is produced as follows. The nucleotide sequence for the cDNA encoding a fusion protein CAR, comprising the amino acid sequences of h679-scFv, CD8α hinge, CD28 TM, CD28 ICD, 4-1BB ICD, and CD3ζ ICD linked in tandem (h679-28-BB-z, FIG. 1) is synthesized by standard techniques, PCR-amplified, and ligated into pCLPS, a third generation self-inactivating lentiviral vector based on pRRL-SIN-CMV-eGFP-WPRE (Dull et al, 1998, J Virol 72: 8463-71), or pELNS (Carpenito et al, 2009, Proc Natl Acad Sci USA 106:3360-5), which differs from pCLPS by replacing CMV with EF-1α as the promoter for transgene expression. The encoded CAR comprises an h679 scFv for binding to HSG.

In-DTPA-Binding CAR

The lentiviral vector for expressing the CAR comprising h734-scFv, CD8α hinge, CD28 TM, CD28 ICD, 4-1BB ICD, and CD3ζ ICD linked in tandem (h734-28-BB-z, FIG. 1) is constructed as described above, except that the nucleotide sequence encoding h679-scFv is replaced with that of h734-scFv.

Anti-Trop-2 CAR

The lentiviral vector for expressing the CAR comprising hRS7-scFv, CD8α hinge, CD28 TM, CD28 ICD, 4-1BB ICD, and CD3ζICD linked in tandem (hRS7-28-BB-z, FIG. 1) is constructed as described above except that the nucleotide sequence encoding h679-scFv is replaced by that of hRS7-scFv.

Anti-CEACAM6 CAR

The lentiviral vector for expressing the CAR comprising hMN-15-scFv, CD8α hinge, CD28 TM, CD28 ICD, 4-1BB ICD, and CD3ζ ICD linked in tandem (hMN-15-28-BB-z FIG. 1) is constructed as described above except that the nucleotide sequence encoding h679-scFv is replaced by that of hMN-15-scFv.

Anti-CEACAM5 CAR

The lentiviral vector for expressing the CAR comprising hMN-14-scFv, CD8α hinge, CD28 TM, CD28 ICD, 4-1BB ICD, and CD3ζ ICD linked in tandem (hMN-14-28-BB-z, FIG. 1) is constructed as described above except that the nucleotide sequence encoding h679-scFv is replaced by that of hMN-14-scFv.

### Example 3

### Construction of Lentiviral Vectors for Expressing Second-Generation CAR

HSG-Binding CAR

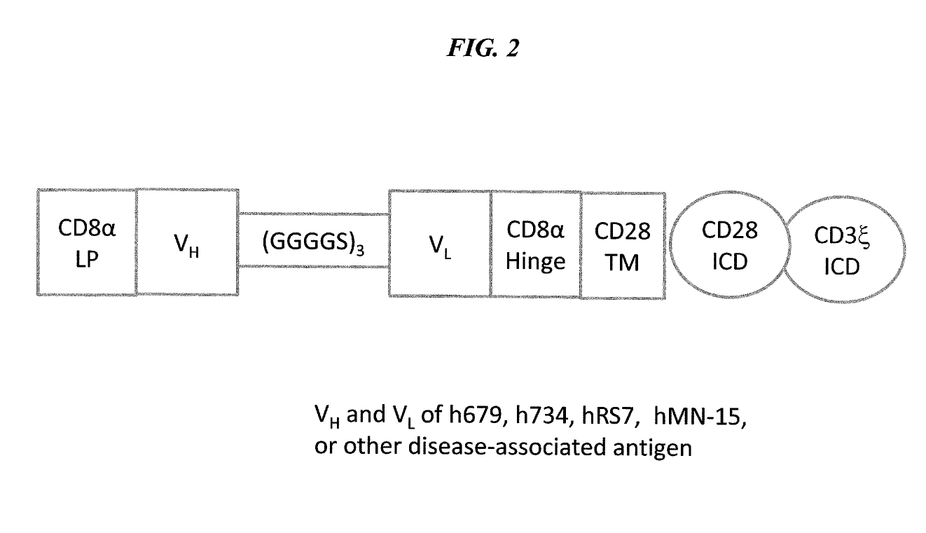

A schematic diagram showing an exemplary second-generation CAR construct is provided in FIG. 2. The CAR construct is produced as follows. The nucleotide sequence for the cDNA encoding the CAR comprising h679-scFv, CD8α hinge, CD28 TM, CD28 ICD, and CD3ζ ICD linked in tandem (h679-28-z, FIG. 2) is synthesized by standard techniques, PCR-amplified, and ligated into pELNS, a self-inactivating lentiviral vector based on pRRL-SIN-CMV-eGFP-WPRE (Dull et al, 1998, J Virol 72: 8463-71), in which transgene expression is driven by the EF-1α promoter.

In-DTPA-Binding CAR

The lentiviral vector for expressing the CAR comprising h734-scFv, CD8α hinge, CD28 TM, CD28 ICD, and CD3ζ ICD linked in tandem (h734-28-z, FIG. 2) is constructed as described above, except that the nucleotide sequence encoding h679-scFv is replaced by that of h734-scFv.

Anti-Trop-2 CAR

The lentiviral vector for expressing the CAR comprising hRS7-scFv, CD8α hinge, CD28 TM, CD28 ICD, and CD3ζ ICD linked in tandem (hRS7-28-z, FIG. 2) is constructed as described above, except that the nucleotide sequence encoding h679-scFv is replaced by that of hRS7-scFv.

Anti-CEACAM6 CAR

The lentiviral vector for expressing the CAR comprising hMN-15-scFv, CD8α hinge, CD28 TM, CD28 ICD, and CD3ζ ICD linked in tandem (hMN-15-28-z, FIG. 2) is constructed as described above, except that the nucleotide sequence encoding h679-scFv is replaced by that of hMN-15-scFv.

### Example 4

### Production of Lentiviral Particles

High-titer, replication-defective lentiviral vectors constructed as described in the Examples above are produced and concentrated as described by Parry R V et al. (2003, J Immunol, 171: 166-74). Briefly, HEK 293T cells (ATCC CRL-3216) are cultured in RPMI 1640, 10% heat-inactivated FCS, 2 mM glutamine, 100 U/mL penicillin, and 100 μg/mL streptomycin sulfate. Cells are seeded at 5×10⁶ per T 150 tissue culture flask 24 h before transfection with 7 μg of pMDG.1 (VSV-G envelop), 18 μg of pRSV.rev (HIV-1 Rev encoding plasmid), 18 μg of pMDLg/p.RRE (packaging plasmid), and 15 μg of the lentiviral vector of interest using Fugene 6 (Roche Molecular Biochemicals). Media are changed 6 h after transfection and the viral supernatant is harvested at 24 and 48 h posttransfection. Viral particles are concentrated 10-fold by ultracentrifugation for 3 h at 28,000 rpm with a Beckman SW28 rotor.

### Example 5

### Transduction of T Cells

For certain purposes, T cells from normal individuals may be used with the subject CAR constructs for construct testing and design. Primary human CD4+ and CD8+ T cells are isolated from the PBMCs of healthy volunteer donors following leukapheresis by negative selection with RosetteSep kits (Stem Cell Technologies). T cells are cultured in complete media (RPMI 1640 supplemented with 10% heat-inactivated FCS, 2 mM glutamine, 100 U/mL penicillin, 100 μg/mL streptomycin sulfate, and 10 mM HEPES), stimulated with monoclonal anti-CD3 and anti-CD28 coated beads for 12 to 24 h, and transduced with a lentiviral vector of interest at MOI (multiplicity of infection) of 5 to 10. Human recombinant IL-2 is added every other day to a 50 U/mL final concentration and a cell density of 0.5 to 1.0×10⁶/mL is maintained.

### Example 6

### Generation and Assessment of Autologous CAR-T Cells from Cancer Patients

The method as described by Brentjens et al (2013, Sci Transl Med 5:177ra38) is followed. Briefly, PBMCs are obtained from cancer patients by leukapheresis, washed, and cryopreserved. T cells are isolated from thawed leukapheresis product, activated with Dynabeads Human T-Activator CD3/CD28 magnetic beads (Invitrogen), and transduced with a lentiviral vector of interest. Transduced T cells are further expanded with the WAVE bioreactor to achieve the desired modified T cell dose.

Modified T cells are assessed for persistence in patient peripheral blood and bone marrow by FACS, anti-tumor activity by in vitro killing of antigen-positive cancer cells, and cytokine profiles by analyzing serial serum samples obtained before and after infusion of modified T cells with the Luminex IS 100 System and commercially available 39-plex cytokine detection assays (Brentjens R L et al., 2011, Blood 118:4817-28).

### Example 7

### Generation of Allogeneic CAR-T Cells from Unrelated Third-Party Donors

PBMCs are obtained from unrelated third-party donors by leukapheresis, washed, and cryopreserved. T cells are isolated from thawed leukapheresis product, and the TCRα constant (TRAC) gene is inactivated using Transcription Activator-Like Effector Nuclease (TALEN™) gene editing technology (Cellectis) to generate TCR-deficient T cells, which are activated with Dynabeads Human T-Activator CD3/CD28 magnetic beads (Invitrogen), and transduced with a lentiviral vector of interest. Transduced TCR-deficient T cells are further expanded with the WAVE bioreactor to achieve the desired modified T cell dose.

### Example 8

### Preparation of HSG-Conjugated IgG

In certain embodiments, an antibody, such as an anti-TAA antibody, may be conjugated to a hapten, such as HSG or In-DTPA, and used for indirect targeting of CAR-T or CAR-NK cells to tumors or other disease targets. The hapten-labeled antibody is allowed to localize to target (e.g., tumor) cells. Then a CAR-T or CAR-NK construct containing a binding site for the hapten is administered to the patient and co-localizes with the hapten-labeled antibody. The advantage to this approach is that the same CAR-T or CAR-NK construct may be targeted to a wide variety of different target cell antigens, merely by changing the specificity of the antibody labeled with hapten.

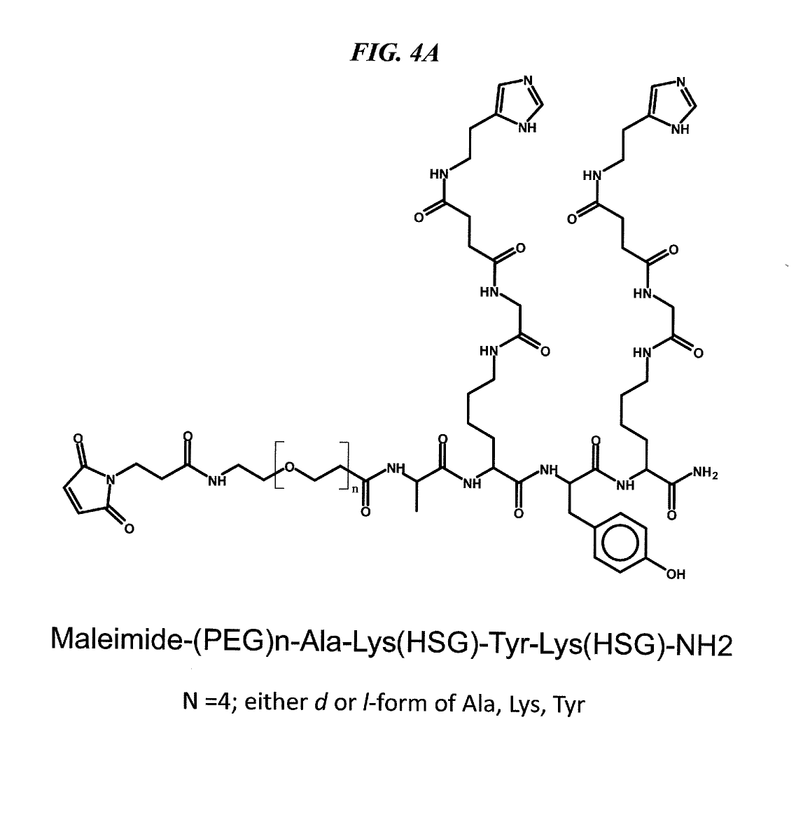

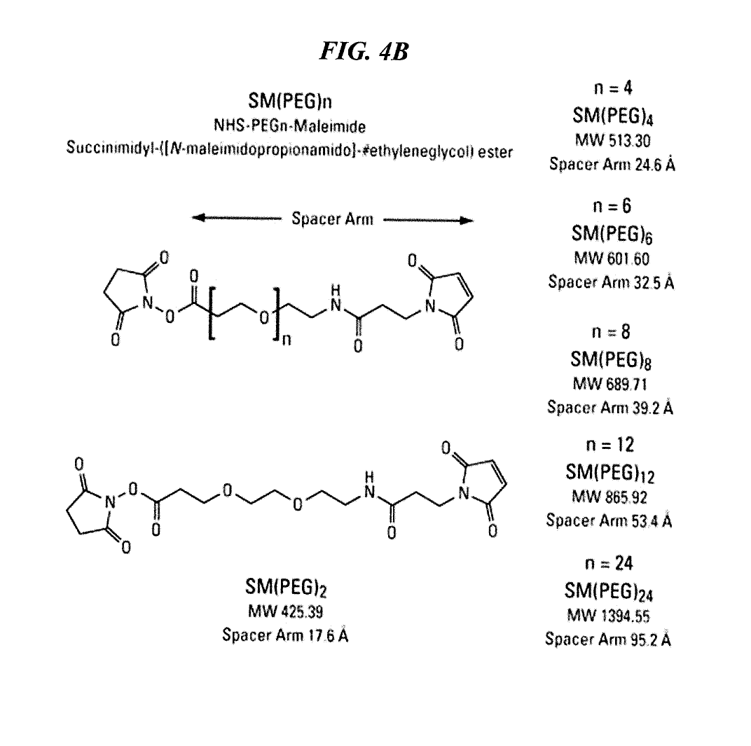

To produce an antibody labeled with the HSG hapten, humanized monoclonal IgG1 is mildly reduced with TCEP in 75 mM sodium acetate buffer (pH 6.5), followed by in situ conjugation at room temperature for 20 min to 10-15-fold molar excess of maleimide-PEG₄-Ala-dLys(HSG)-dTyr-dLys(HSG)-NH₂ (FIG. 4A), and purified using a desalting column. The di-HSG moiety is prepared by reacting SM(PEG)₄, a crosslinking agent of the SM(PEG)n family (FIG. 4B) available from Thermo Scientific, with Ala-dLys(HSG)-dTyr-dLys(HSG)-NH₂.

### Example 9

### Preparation of DTPA-In-Conjugated IgG

A humanized monoclonal IgG1 is mildly reduced with TCEP in 75 mM sodium acetate buffer (pH 6.5), followed by in situ conjugation at room temperature for 20 min to 10-15-fold molar excess indium-complexed maleimide-PEG₄-dPhe-dLys(DTPA)-dTyr-dLys(DTPA)-NH₂, and purified using a desalting column. The di-DTPA-In-moiety is prepared by reacting SM(PEG)₄ (FIG. 4B) with dPhe-dLys(DTPA)-dTyr-dLys(DTPA)-NH₂.

### Example 10

### Therapy of Trop-2-Positive Human Cancer Xenografts Using CAR-T Cells Transduced to Express hRS7-28-BB-z

A Trop-2-positive xenograft model is established by implanting BxPC-3 pancreatic cancer cells in the flanks of NOG mice. After the tumor volume reaches ˜500 mm³, the mice are treated with two intratumoral injections of 15×10⁶ CAR-T cells (˜70 to 80% transgene positive) one week apart. A potent antitumor effect is observed in all mice receiving the relevant CAR-T cells, but not the irrelevant CAR-T cells.

### Example 11

### Therapy of CEACAM5-Positive Human Cancer Xenografts Via Sequential Targeting of HSG-Conjugated hMN-14 IgG and CAR-T Cells Transduced to Express h679-28-BB-z

A CEACAM5-positive xenograft model is established by implanting BxPC-3 pancreatic cancer cells in the flanks of NOG mice. After the tumor volume reaches ˜250 mm³, the mice are injected i.v. with HSG-conjugated hMN-14 IgG, followed by intratumoral injections of 15×10⁶ CAR-T cells (˜70 to 80% transgene positive) on day-3 and day-10. A potent antitumor effect is observed in all mice receiving the sequential treatment, but not in mice receiving only the CAR-T cells.

### Example 12

### Therapy of CEACAM5-Positive Human Cancer Xenografts by Predosing with Unconjugated hMN-14 IgG, Followed by Sequential Targeting of HSG-Conjugated hMN-14 IgG and CAR-T Cells Transduced to Express h679-28-BB-z

A CEACAM5-positive xenograft model is established by implanting BxPC-3 pancreatic cancer cells in the flanks of NOG mice. After the tumor volume reaches ˜250 mm³, the mice are separated into two groups. One group receives a predose of 12.5 mg/kg of unconjugated hMN-14 IgG 1 day prior to the administration of HSG-conjugated hMN-14 IgG, followed by intravenous injections of 15×10⁶ HSG-binding CAR-T cells (˜70 to 80% transgene positive) on day-3 and day-10. The other group receives the same treatment except the predosing step is omitted. A potent antitumor effect is observed in both groups, which indicates that predosing does not affect the subsequent targeting of CAR-T to CEA-expressing tumor tagged with HSG-conjugated hMN-14. Predosing protects normal tissues that express CEACAM5 and decreases systemic toxicity of the CAR-T administration.

### Example 13

### Generation of Genetically Engineered NK Cells with HSG-Binding CAR

NK cells amenable to genetic engineering with HSG-binding CAR or other CAR of interest include primary NK cells and several NK-like human cell lines such as NK-92 (Gong et al., Leukemia 8: 652-8, 1994), NK-92MI (Tam et al., Hum Gene Ther 10: 1359-73, 1999), NK-92fc (Binyamin et al., J Immunol 180: 6392-6401, 2008), NKL (Robertson et al., Exp Hematol 24: 406-15, 1996), NKG (Cheng et al., Cell transplant 20: 1731-46, 2011), NK-YS (Tsuchiyama et al., Blood 92: 1374-83, 1998), KHYG-1 (Yagita et al., Leukemia 14: 922-30, 2000), and YT (Yodoi et al., J Immunol 134: 1623-30, 1985).

Transduction of Primary NK Cells by mRNA Electroporation.

PBMCs are obtained from healthy donors by leukapheresis, washed, and cryopreserved until use. Primary NK cells are purified by depleting non-NK cells from thawed PBMCs using a Miltenyi NK cell isolation kit (Auburn, Calif.), expanded, and transfected with the mRNA transcribed from the transgene encoding HSG-binding CAR of Example 2 by electroporation (100 μg/mL per 1 to 3×10⁸ cells/mL) as described by Li et al (Cancer Gene Ther 17: 147-54, 2010). Immediately after electroporation, cells are recovered from the processing chamber, placed at 37° C., 5% CO₂ for 20 min, resuspended in RPMI-1640 media with 10% FBS and 100 IU/mL IL-2, and cultured at 37° C., 5% CO2 until analysis for the expression of HSG-binding CAR, viability, IFN-γ production, and cytotoxicity.

Transduction of NK-92 Cells by Lentiviral Vector.

The NK-92 cell line is purchased from ATCC (CRL-2407) and maintained in MyeloCult medium (Stem Cell Technology, Vancouver, Canada) supplemented with 500 U/mL Proleukin (Chiron, Emeryville, Calif.). NK-92 cells are transduced with p-CLPS-h679-28-BB-z (Example 2) using the spinfection protocol as described by Boissel et al (Leuk Lymphoma 53: 958-65, 2012), Transduced cells are expanded in MyeloCult medium supplemented with 1000 U/mL Proleukin for 48 to 72 h and analyzed for transduction efficiency, expression of HSG-binding CAR, and cytotoxicity.

CAR-NK Therapy.

CEACAM5-positive colorectal cancer patients are predosed with 12.5 mg/kg of unconjugated hMN-14 IgG 1 day prior to the administration of HSG-conjugated hMN-14 IgG, followed by intravenous injections of 5×10⁷ HSG-binding CAR-NK cells per kg (˜70 to 80% transgene positive) on day-3 and day-10. A potent antitumor effect is observed, indicating that predosing does not affect the subsequent targeting of CAR-NK to CEA-expressing tumor tagged with HSG-conjugated hMN-14. Predosing protects normal tissues that express CEACAM5 and decreases systemic toxicity of the CAR-T administration.

Additional CAR sequences for NK cell transfection are shown below.

HSG-targeting 2^{nd}-generation CAR for NK-92
(491 AAs)
SP_{CD8α}-VKₕ₆₇₉-(GGGGS)3-VHₕ₆₇₉-Hinge_{CD8α}-TM_{CD8α}-
ICD_{4-1BB}-ICD_{CD3ξ}(“(GGGGS)₃”disclosed as SEQ ID
NO: 18)

(SEQ ID NO: 21)

MALPVTALLLPLALLLHAARPDIQLTQSPSSLAVSPGERVTLTCKSSQSL
FNSRTRKNYLGWYQQKPGQSPKLLIYWASTRESGVPDRFSGSGSGTDFTL
TINSLQAEDVAVYYCTQVYYLCTFGAGTKLEIKRGGGGSGGGGSGGGGSQ
VQLQESGGDLVKPGGSLKLSCAASGFTFSIYTMSWLRQTPGKGLEWVATL
SGDGDDIYYPDSVKGRFTISRDNAKNSLYLQMNSLRAEDTALYYCARVRL
GDWDFDVWGQGTTVTVSSTTTPAPRPPTPAPTIASQPLSLRPEACRPAAG
GAVHTRGLDFACDIYIWAPLAGTCGVLLLSLVITLYCKRGRKKLLYIFKQ
PFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR
HSG-targeting 3^{rd} generation CAR for
NK-92MI (537 AAs)
SP_{CD8α}-VKₕ₆₇₉-(GGGGS)3-VHₕ₆₇₉-Hinge_{CD8α}-TM_{CD28}-
ICD_{CD28}-ICD_{4-1BB}-ICD_{CD3ξ}(“(GGGGS)₃” disclosed as
SEQ ID NO: 18)

(SEQ ID NO: 22)

MALPVTALLLPLALLLHAARPDIQLTQSPSSLAVSPGERVTLTCKSSQSL
FNSRTRKNYLGWYQQKPGQSPKLLIYWASTRESGVPDRFSGSGSGTDFTL
TINSLQAEDVAVYYCTQVYYLCTFGAGTKLEIKRGGGGSGGGGSGGGGSQ
VQLQESGGDLVKPGGSLKLSCAASGFTFSIYTMSWLRQTPGKGLEWVATL
SGDGDDIYYPDSVKGRFTISRDNAKNSLYLQMNSLRAEDTALYYCARVRL
GDWDFDVWGQGTTVTVSSTTTPAPRPPTPAPTIASQPLSLRPEACRPAAG
GAVHTRGLDFACDFWVLVVVGGVLACYSLLVTVAFIIFWVRSKRSRLLHS
DYMNMTPRRPGPTRKHYQPYAPPRDFAAYRSIDKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR

### Example 14

### Use of ADC (IMMU-132 or hRS7-SN-38) to Treat Therapy-Refractive Metastatic Colonic Cancer (mCRC)

The patient was a 62-year-old woman with mCRC who originally presented with metastatic disease in January 2012. She had laparoscopic ileal transverse colectomy as the first therapy a couple of weeks after diagnosis, and then received 4 cycles of FOLFOX (leucovorin, 5-fluorouracil, oxaliplatin) chemotherapy in a neoadjuvant setting prior to right hepatectomy in March 2012 for removal of metastatic lesions in the right lobe of the liver. This was followed by an adjuvant FOLFOX regimen that resumed in June, 2012, for a total of 12 cycles of FOLFOX. In August, oxaliplatin was dropped from the regimen due to worsening neurotoxicity. Her last cycle of 5-FU was on 09/25/12.

CT done in January 2013 showed metastases to liver. She was then assessed as a good candidate for enrollment to IMMU-132 (hRS7-SN-38) investigational study. Comorbidities in her medical history include asthma, diabetes mellitus, hypertension, hypercholesteremia, heart murmur, hiatal hernia, hypothyroidism, carpel tunnel syndrome, glaucoma, depression, restless leg syndrome, and neuropathy. Her surgical history includes tubo-ligation (1975), thyroidectomy (1983), cholescystectomy (2001), carpel tunnel release (2008), and glaucoma surgery.

At the time of entry into this therapy, her target lesion was a 3.1-cm tumor in the left lobe of the liver. Non-target lesions included several hypo-attenuated masses in the liver. Her baseline CEA was 781 ng/mL.

IMMU-132 was given on a once-weekly schedule by infusion for 2 consecutive weeks, then a rest of one week, this constituting a treatment cycle. These cycles were repeated as tolerated. The first infusion of IMMU-132 (8 mg/kg) was started on Feb. 15, 2013, and completed without notable events. She experienced nausea (Grade 2) and fatigue (Grade 2) during the course of the first cycle and has been continuing the treatment since then without major adverse events. She reported alopecia and constipation in March 2013. The first response assessment done (after 6 doses) on Mar. 8, 2013 showed a shrinkage of target lesion by 29% by computed tomography (CT). Her CEA level decreased to 230 ng/mL on Mar. 25, 2013. In the second response assessment (after 10 doses) on May 23, 2013, the target lesion shrank by 39%, thus constituting a partial response by RECIST criteria. She has been continuing treatment, receiving 6 cycles constituting 12 doses of hRS7-SN-38 (IMMU-132) at 8 mg/kg. Her overall health and clinical symptoms improved considerably since starting this investigational treatment.

### Example 15

### ADC Therapy with IMMU-132 for Metastatic Solid Cancers

IMMU-132 is an ADC comprising the active metabolite of CPT-11, SN-38, conjugated by a pH-sensitive linker (average drug-antibody ratio=7.6) to the hRS7 anti-Trop-2 humanized monoclonal antibody, which exhibits rapid internalization when bound to Trop-2. IMMU-132 targets Trop-2, a type I transmembrane protein expressed in high prevalence and specificity by many carcinomas. This Example reports a Phase I clinical trial of 25 patients with different metastatic cancers (pancreatic, 7; triple-negative breast [TNBC], 4; colorectal [CRC], 3; gastric, 3, esophageal, prostatic, ovarian, non-small-cell lung, small-cell lung [SCLC], renal, tonsillar, urinary bladder, 1 each) after failing a median of 3 prior treatments (some including topoisomerase-I and -II inhibiting drugs).

IMMU-132 was administered in repeated 21-day cycles, with each treatment given on days 1 and 8. Dosing started at 8 mg/kg/dose (i.e., 16 mg/kg/cycle), and escalated to 18 mg/kg before encountering dose-limiting neutropenia, in a 3+3 trial design. Fatigue, alopecia, and occasional mild to moderate diarrhea were some of the more common non-hematological toxicities, with 2 patients also reporting a rash. Over 80% of 24 assessable patients had stable disease or tumor shrinkage (SD and PR) among the various metastatic cancers as best response by CT. Three patients (CRC, TNBC, SCLC) have PRs by RECIST; median TTP for all patients, excluding those with pancreatic cancer, is >18 weeks. Neutropenia has been controlled by dose reduction to 8-10 mg/kg/dose (16-20 mg/kg/cycle).

Immunohistochemistry showed strong expression of Trop-2 in most archived patient tumors, but it was not detected in serum. Corresponding reductions in blood tumor marker titers (e.g., CEA, CA19-9) reflected tumor responses. No anti-antibody or anti-SN-38 antibodies have been detected despite repeated dosing. Peak and trough assessments of IMMU-132 concentrations in the serum show that the conjugate cleared completely within 7 days, an expected finding based on in vitro studies showing 50% of the SN-38 is released in the serum every day. These results indicate that this novel ADC, given in doses ranging from 16-24 mg/kg per cycle, shows a high therapeutic index in diverse metastatic solid cancers.

### Example 16

### IMMU-130, an SN-38 ADC that Targets CEACAM5, is Therapeutically Active in Metastatic Colorectal Cancer (mCRC)

IMMU-130, an ADC of SN-38 conjugated by a pH-sensitive linker (7.6 average drug-antibody ratio) to the humanized anti-CEACAM5 antibody (labetuzumab), is completing two Phase I trials. In both, eligible patients with advanced mCRC were required to have failed/relapsed standard treatments, one being the topoisomerase-I inhibiting drug, CPT-11 (irinotecan), and an elevated plasma CEA (>5 ng/mL).

IMMU-130 was administered every 14 days (EOW) at doses starting from 2.0 mg/kg in the first protocol (IMMU-130-01). Febrile neutropenia occurred in 2 of 3 patients at 24 mg/kg; otherwise at ≦16 mg/kg, neutropenia (≧Grade 2) was observed in 7 patients, with one also experiencing thrombocytopenia. One patient [of 8 who received ≧4 doses (2 cycles)] showed a 40.6% decrease in liver (starting at 7 cm) and lung target lesions (PR by RECIST) for 4.7 months, with no major toxicity, tolerating a total of 18 doses at 16 mg/kg. The study expanded at 12 mg/kg EOW.

Since SN-38 is most effective in S-phase cells, a more protracted exposure could improve efficacy. Thus, in a second Phase I trial (IMMU-130-02), dosing was intensified to twice-weekly, starting at 6 mg/kg/dose for 2 weeks (4 doses) with 1 week off, as a treatment cycle, in a 3+3 trial design. Neutropenia and manageable diarrhea were the major side effects, until dose reduction to 4.0 mg/kg twice-weekly, with early results indicating multiple cycles are well-tolerated. Currently, tumor shrinkage occurred in 3 patients, with 1 in continuing PR (−46%) by RECIST, among 6 patients who completed ≧4 doses (1 cycle). In both trials, CEA blood titers correlated with tumor response, and high levels did not interfere with therapy. There have been no anti-antibody or anti-SN-38 antibody reactions, based on ELISA tests. In each study, the ADC was cleared by 50% within the first 24 h, which is much longer exposure than with typical doses of the parental molecule, CPT-11. These results indicate that this novel ADC, given in different regimens averaging ˜16-24 mg/kg/cycle, shows a high therapeutic index in advanced mCRC patients. Since CEACAM-5 has elevated expression in breast and lung cancers, as well as other epithelial tumors, it may be a useful target in other cancers as well.

### Example 17

### Antitumor Activity of Checkpoint Inhibitor Antibody Alone or Combined with CAR-T

To determine if the antitumor activity of the exemplary checkpoint inhibitor antibody, ipilimumab (anti-CTLA4) is synergistic with or inhibited by the addition of CAR-T treatment, CTLA4 mAb is evaluated alone or in combination with the exemplary anti-Trop-2 CAR-T disclosed in Example 2 or Example 3 above. M109 lung carcinoma, SA1N fibrosarcoma, and CT26 colon carcinoma models are chosen based on different sensitivity to the various agents and CTLA4 blockade.

All compounds are tested at their optimal dose and schedule. When used in combination, CTLA4 mAb is initiated one day after the first dose of CAR-T. Percent tumor growth inhibition and number of days to reach target tumor size are used to evaluate efficacy. Antitumor activity is scored as: complete regression (CR; non-palpable tumor) or partial regression (PR; 50% reduction in tumor volume). Synergy is defined as antitumor activity significantly superior (p<0.05) to the activity of monotherapy with each agent.

In the SA1N fibrosarcoma tumor model, which is sensitive to CTLA4 blockade and modestly sensitive to anti-Trop-2 CAR-T, borderline synergy is evident with the combination of CTLA4 mAb and anti-Trop-2 CAR-T. In the M109 lung metastasis model and CT26 colon carcinoma model, synergy is detected for CTLA4 mAb combined with anti-Trop-2 CAR-T.

In summary, addition of CTLA4 mAb to anti-Trop-2 CAR-T results in model-dependent synergistic activities. Synergy is observed regardless of the immunogenicity of the tumor and only when at least one of the therapies is active. The combination regimen is well-tolerated and does not appear to inhibit CTLA4 mAb activity. Synergy is observed in tumors unresponsive to CTLA4 mAb alone, suggesting that the addition of CAR-T might induce immunogenic cell death.

### Example 18

### Combination Therapy with ADC (IMMU-132) and Interferon-α (PEGINTERFERON®) to Treat Refractory, Metastatic, Non-Small Cell Lung Cancer

The patient is a 60-year-old man diagnosed with non-small cell lung cancer. The patient is given chemotherapy regimens of carboplatin, bevacizumab for 6 months and shows a response, and then after progressing, receives further courses of chemotherapy with carboplatin, etoposide, TAXOTERE®, gemcitabine over the next 2 years, with occasional responses lasting no more than 2 months. The patient then presents with a left mediastinal mass measuring 6.5×4 cm and pleural effusion.

After signing informed consent, the patient is given IMMU-132 at a dose of 10 mg/kg on days 1 and 8 of a 21-day cycle. After the first week of treatment, the patient is given combination therapy with IMMU-132 and PEGINTERFERON®. During the first two injections, brief periods of neutropenia and diarrhea are experienced, with 4 bowel movements within 4 hours, but these resolve or respond to symptomatic medications within 2 days. After a total of 6 infusions of IMMU-132 and 5 infusions of PEGINTERFERON®, CT evaluation of the index lesion shows a 22% reduction, just below a partial response but definite tumor shrinkage. The patient continues with this therapy for another three months, when a partial response of 45% tumor shrinkage of the sum of the diameters of the index lesion is noted by CT, thus constituting a partial response by RECIST criteria. The combination therapy appears to provide a synergistic response, compared to the two agents administered separately.

### Example 19

### Combination Therapy with ADC (IMMU-130) and Anti-CEACAM5 CAR-T to Treat Advanced Colonic Cancer

The patient is a 75-year-old woman initially diagnosed with metastatic colonic cancer (Stage IV). She has a right partial hemicolectomy and partial resection of her small intestine and then receives FOLFOX, FOLFOX+bevacizumab, FOLFIRI+ramucirumab, and FOLFIRI+cetuximab therapies for a year and a half, when she shows progression of disease, with spread of disease to the posterior cul-de-sac, omentum, with ascites in her pelvis and a pleural effusion on the right side of her chest cavity. Her baseline CEA titer just before this therapy is 15 ng/mL. She is given 6 mg/kg IMMU-130 (anti-CEACAM5-SN-38) twice weekly for 2 consecutive weeks, and then one week rest (3-week cycle). After the first cycle, the patient is given combination therapy with IMMU-130 and anti-Trop-2 CAR-T, which is administered by continuous infusion on the same 3-week cycle. After 5 cycles, which are tolerated very well, without any major hematological or non-hematological toxicities, her plasma CEA titer shrinks modestly to 1.3 ng/mL, but at the 8-week evaluation she shows a 21% shrinkage of the index tumor lesions, which increases to a 27% shrinkage at 13 weeks. Surprisingly, the patient's ascites and pleural effusion both decrease (with the latter disappearing) at this time, thus improving the patient's overall status remarkably. The combination therapy appears to provide a synergistic response, compared to the two agents administered separately.

### Example 20

### Combination Therapy with ADC (IMMU-130), Anti-Trop-2 CAR-T and Interferon-α to Treat Gastric Cancer Patient with Stage IV Metastatic Disease

The patient is a 52-year-old male who sought medical attention because of gastric discomfort and pain related to eating for about 6 years, and with weight loss during the past 12 months. Palpation of the stomach area reveals a firm lump which is then gastroscoped, revealing an ulcerous mass at the lower part of his stomach. This is biopsied and diagnosed as a gastric adenocarcinoma. Laboratory testing reveals no specific abnormal changes, except that liver function tests, LDH, and CEA are elevated, the latter being 10.2 ng/mL. The patent then undergoes a total-body PET scan, which discloses, in addition to the gastric tumor, metastatic disease in the left axilla and in the right lobe of the liver (2 small metastases). The patient has his gastric tumor resected, and then has baseline CT measurements of his metastatic tumors. Four weeks after surgery, he receives 3 courses of combination chemotherapy consisting of a regimen of cisplatin and 5-fluorouracil (CF), but does not tolerate this well, so is switched to treatment with docetaxel. It appears that the disease is stabilized for about 4 months, based on CT scans, but then the patient complains of further weight loss, abdominal pain, loss of appetite, and extreme fatigue cause repeated CT studies, which show increase in size of the metastases by a sum of 20% and a suspicious lesion at the site of the original gastric resection.

The patient is then given experimental therapy with IMMU-130 (anti-CEACAM5-SN-38) on a weekly schedule of 8 mg/kg. After the first week, combination therapy with IMMU-130, anti-Trop-2 CAR-T and interferon-α is initiated. The patient exhibits no evidence of diarrhea or neutropenia over the following 4 weeks. The patient then undergoes a CT study to measure his metastatic tumor sizes and to view the original area of gastric resection. The radiologist measures, according to RECIST criteria, a decrease of the sum of the metastatic lesions, compared to baseline prior to therapy, of 23%. There does not seem to be any clear lesion in the area of the original gastric resection. The patient's CEA titer at this time is 7.2 ng/mL, which is much reduced from the baseline value of 14.5 ng/mL. The patient continues on weekly combination therapy, and after a total of 13 infusions, his CT studies show that one liver metastasis has disappeared and the sum of all metastatic lesions is decreased by 41%, constituting a partial response by RECIST. The patient's general condition improves and he resumes his usual activities while continuing to receive maintenance therapy every third week. At the last measurement of blood CEA, the value is 4.8 ng/mL, which is within the normal range for a smoker, which is the case for this patient.

### Example 21

### Combination of Anti-HLA-DR Antibody and Anti-CEACAM5 CAR-T to Treat Advanced Colonic Cancer

The patient is a 70-year-old man initially diagnosed with metastatic colonic cancer (Stage IV). He has a right partial hemicolectomy and partial resection of his small intestine and then receives FOLFOX, FOLFOX+bevacizumab, FOLFIRI+ramucirumab, and FOLFIRI+cetuximab therapies for a year and a half, when he shows progression of disease, with spread of disease to the posterior cul-de-sac, omentum, with ascites in his pelvis and a pleural effusion on the right side of his chest cavity. His baseline CEA titer just before this therapy is 15 ng/mL. He is given anti-Trop-2 CAR-T, which is administered by continuous infusion twice weekly for 2 consecutive weeks, and then one week rest (3-week cycle). After the first administration of CAR-T in each cycle, a dose of 5 mg/kg of the anti-HLA DR hL243 antibody is administered to prevent development of a cytokine storm. After 5 cycles, which well tolerated, without any major hematological or non-hematological toxicities, his plasma CEA titer decreases to 1.3 ng/mL. At the 8-week evaluation he shows a 31% shrinkage of the index tumor lesions, which increases to a 40% shrinkage at 13 weeks. Surprisingly, the patient's ascites and pleural effusion both decrease (with the latter disappearing) at this time, thus improving the patient's overall status remarkably. The use of anti-HLA-DR antibody is effective to prevent immunotoxicities induced by CAR-T administration.

### Example 22

### Generation of Genetically Engineered NK Cells with HSG-Binding CAR

In certain embodiments, the CAR-NK or CAR-T cells may be engineered with an antibody moiety that binds a hapten, such as HSG. The HSG-binding moiety may be used to target cells that have been previously tagged with a hapten-labeled antibody. In this way, a single CAR construct may be targeted to multiple target cells expressing different antigens, by using different HSG-labeled antibodies to tag the appropriate target cell.

NK cells amenable to genetic engineering with HSG-binding CAR or other CAR of interest include primary NK cells and several NK-like human cell lines such as NK-92 (Gong et al., Leukemia 8: 652-8, 1994), NK-92MI (Tam et al., Hum Gene Ther 10: 1359-73, 1999), NK-92fc (Binyamin et al., J Immunol 180: 6392-6401, 2008), NKL (Robertson et al., Exp Hematol 24: 406-15, 1996), NKG (Cheng et al., Cell transplant 20: 1731-46, 2011), NK-YS (Tsuchiyama et al., Blood 92: 1374-83, 1998), KHYG-1 (Yagita et al., Leukemia 14: 922-30, 2000), and YT (Yodoi et al., J Immunol 134: 1623-30, 1985).

Transduction of Primary NK Cells by mRNA Electroporation

PBMCs are obtained from healthy donors by leukapheresis, washed, and cryopreserved until use. Primary NK cells are purified by depleting non-NK cells from thawed PBMCs using a Miltenyi NK cell isolation kit (Auburn, Calif.), expanded, and transfected with the mRNA transcribed from the transgene encoding HSG-binding CAR by electroporation (100 μg/mL per 1 to 3×10⁸ cells/mL) as described by Li et al (Cancer Gene Ther 17: 147-54, 2010). Immediately after electroporation, cells are recovered from the processing chamber, placed at 37° C., 5% CO2 for 20 min, resuspended in RPMI-1640 media with 10% FBS and 100 IU/mL IL-2, and cultured at 37° C., 5% CO2 until analysis for the expression of HSG-binding CAR, viability, IFN-γ production, and cytotoxicity.

Transduction of NK-92 Cells by Lentiviral Vector

The NK-92 cell line is purchased from ATCC (CRL-2407) and maintained in MyeloCult medium (Stem Cell Technology, Vancouver, Canada) supplemented with 500 U/mL Proleukin (Chiron, Emeryville, Calif.). NK-92 cells are transduced with p-CLPS-h679-28-BB-z (Example 2) using the spinfection protocol as described by Boissel et al (Leuk Lymphoma 53: 958-65, 2012), Transduced cells are expanded in MyeloCult medium supplemented with 1000 U/mL Proleukin for 48 to 72 h and analyzed for transduction efficiency, expression of HSG-binding CAR, and cytotoxicity.

### Example 23

### Design and Construction of hRS7-CAR

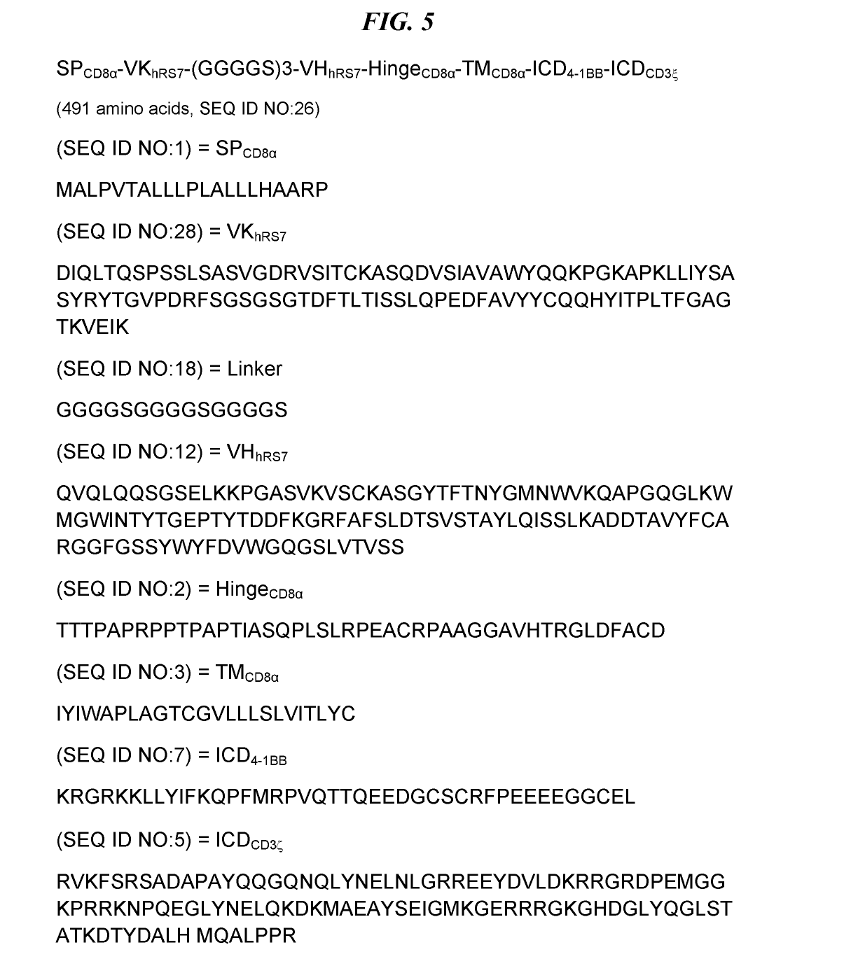

A schematic diagram showing the design of hRS7-CAR, a human Trop-2-targeting CAR, is provided below, with the corresponding amino acid sequence provided in FIG. 5.

SP_{CD8α}-VK_{hRS7}-(GGGGS)3-VH_{hRS7}-Hinge_{CD8α}-TM_{CD8α}-
ICD_{4-1BB}-ICD_{CD3} (“(GGGGS)₃”disclosed as SEQ ID
NO: 18)

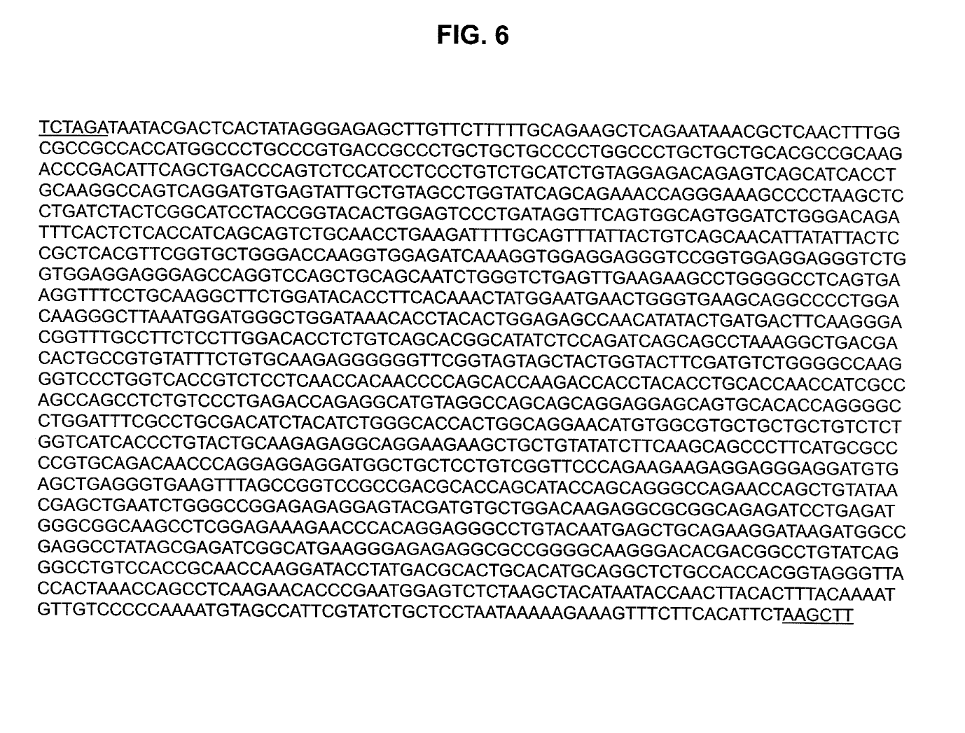

The hRS7-CAR construct consists of the CD8α signal peptide sequence, the V_{K} and V_{H} of hRS7 (a humanized anti-human Trop-2 mAb), the hinge region and transmembrane domain of CD8α, intracellular domain of 4-1BB, and intracellular domain of CD3ξ. A schematic diagram showing the DNA template for in vitro synthesis of hRS7-CAR mRNA is provided below, with the corresponding nucleotide sequence provided in FIG. 6.

Xba I-T7 Promoter-5′-UTR-Kozak Sequence-hRS7-CAR-3′-UTR-Hind III

The template comprises the DNA sequence encoding hRS7-CAR, which is added to the 5′end, a T7 promoter, a 5′-untranslated region (UTR) sequence of human globin gene, and a Kozak sequence, and to the 3′end, a 3′-UTR sequence of human globin gene. To facilitate cloning, the Xba I and Hind III restriction sites are added to the 5′ and 3′ ends, respectively. All DNA sequences were synthesized by Genscript (Piscataway, N.J.).

Synthesis of hRS7-CAR mRNA

The DNA template for hRS7-CAR was cloned into Xba I and Hind III sites of PUC57. The resulting vector (PUC57-hRS7-CAR) was linearized at the Hind III site, and in vitro mRNA synthesis was performed using the mMESSAGE mMACHINE® T7 Ultra Kit (Thermo Fisher Scientific, Carlsbad, Calif.) according to the manufacturer's instructions. This kit couples in vitro transcription with 5′-capping and 3′-polyadenylation in order to increase mRNA stability and translation. The yield was determined by Nanodrop UV-Vis Spectrophotometer (Thermo Scientific, Wilmington, Del.), and the integrity of the final mRNA products was examined by gel electrophoresis, which showed essentially a single band (not shown).

Lentiviral Vector Construction

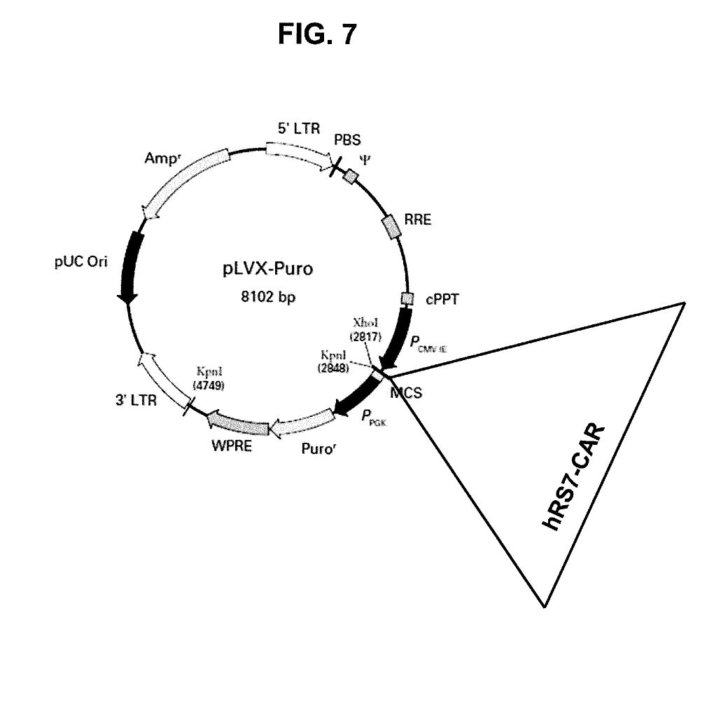

The DNA sequence encoding hRS7-CAR was amplified from PUC57-hRS7-CAR by PCR using a high-fidelity Phusion DNA polymerase (New England Biolabs, Ipswich, Mass.) and the following primers: Forward: 5′-TCAACTCGAGCGCCGCCACCATGGCC-3′ (SEQ ID NO: 24), Reverse: 5′-CTGGTCTAGAGGTAACCCTACCGTGGTGG-3′ (SEQ ID NO: 25). The PCR product was cloned into the pLVX-puro vector (Clontech Laboratories, Mountain View, Calif.) at the restriction sites XhoI and XbaI, and the resulting vector (pLVX-puro-hRS7-CAR) sequenced for accuracy. A schematic of pLVX-puro-hRS7-CAR is provided in FIG. 7.

The new construct of pLVX-Puro-hRS7-CAR (1493 bp) was verified by digestion with restriction enzymes of XbaI and XhoI and gel electrophoresis (not shown), and sequenced after maxiprep.

### Example 24

### Generation of hRS7-CAR-NK-92MI Using mRNA Electroporation Electroporation of hRS7-CAR mRNA

NK-92MI cells were grown to log phase in Myelocult medium (STEMCELL Technologies, Vancouver, Canada), washed and resuspended in serum-free MEM medium (Thermo Fisher Scientific) at a concentration of 1.67×10⁷ cells/ml. A mixture of 1×10⁷ cells in 600 μl MEM medium and 30 μg mRNA in 100 μl water was transferred into a 4-mm electroporation cuvette (BioRad, Hercules, Calif.). After incubation on ice for 10 min, electroporation was performed using the conditions of 300 V, 150 μF, and 200 [symbol] . Cells were incubated for another 10 min, then transferred back into Myelocult medium and cultured at 37° C. and 5% CO₂ for 24 to 48 h before analysis for the expression of hRS7 by WU, a rat anti-id mAb to hRS7.

Expression of hRS7-CAR on hRS7-CAR-NK-92MI

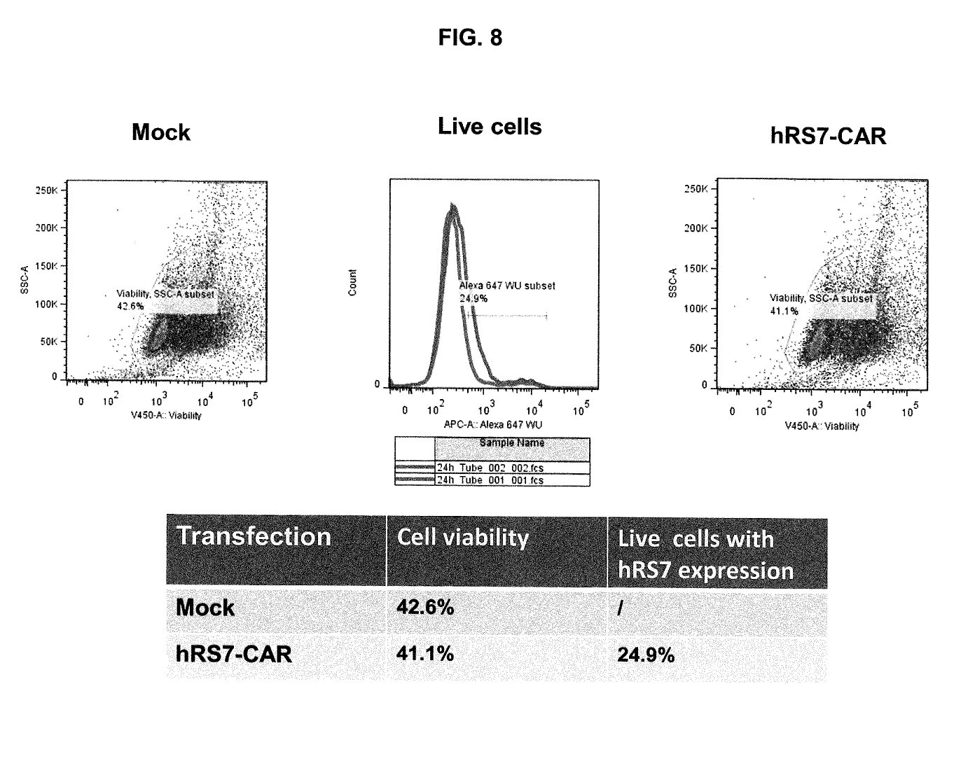

NK-92MI cells were transfected with hRS7-CAR mRNA or with buffer only (mock). Total protein was extracted with RIPA buffer, separated on SDS-PAGE, and probed with WU-HRP by Western blot (not shown). A distinct band of about 50 kDa was observed for the cell lysates of NK-92MI transfected with hRS7-CAR mRNA, but not for the mock-transfected NK-92MI. As the calculated molecular weight of hRS7-CAR is about 51 kDa, these results confirm that hRS7-CAR was produced in NK-92MI cells transfected with hRS7-CAR mRNA. The expression of hRS7 on the cell surface of live hRS7-CAR-NK-92MI was also demonstrated by flow cytometry in FIG. 8, which shows about 41% of NK-92MI cells transfected with hRS7-CAR by electroporation to be alive at the time of analysis and 25% of this subpopulation to express hRS7.

### Example 25

### Cytotoxicity Assay by MTS

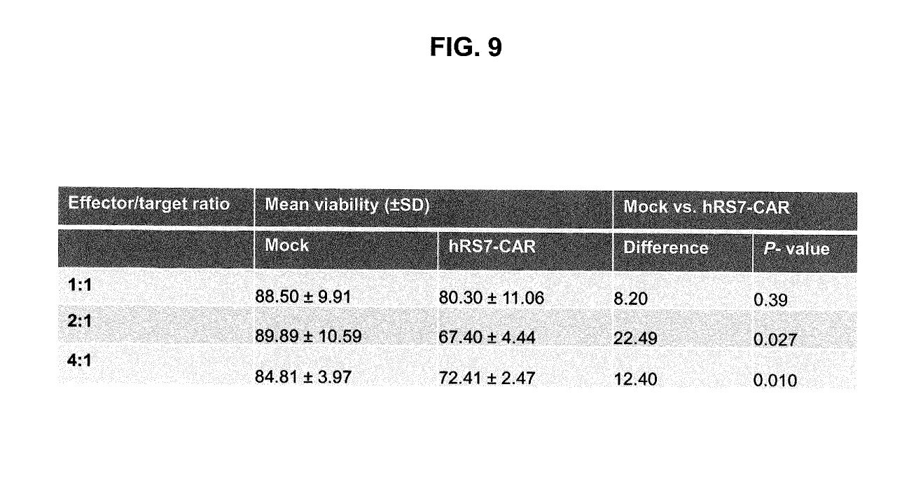

NK-92MI cells were transfected with and without (mock) hRS7-CAR mRNA. After 24-h incubation, they were mixed with Trop-2-expressing HCC1806 (4,500 cells/well) in a 96-well plate at three different effector to target ratios (1:1, 2:1, or 4:1), and incubated overnight. On the next day, NK-92MI and dead HCC1806 cells, both being non-adherent, were washed off. The adhered, vital HCC1806 cells were cultured for two additional days, and the viability was determined by MTS assay. The results summarized in FIG. 9 indicate NK-92MI cells transfected with hRS7-CAR mRNA significantly killed more HCC1806 cells at the effector to target ratio of 2:1 or 4:1, in comparison to mock-transfected NK-92MI.

### Example 26

### Cytotoxicity by Flow Cytometry

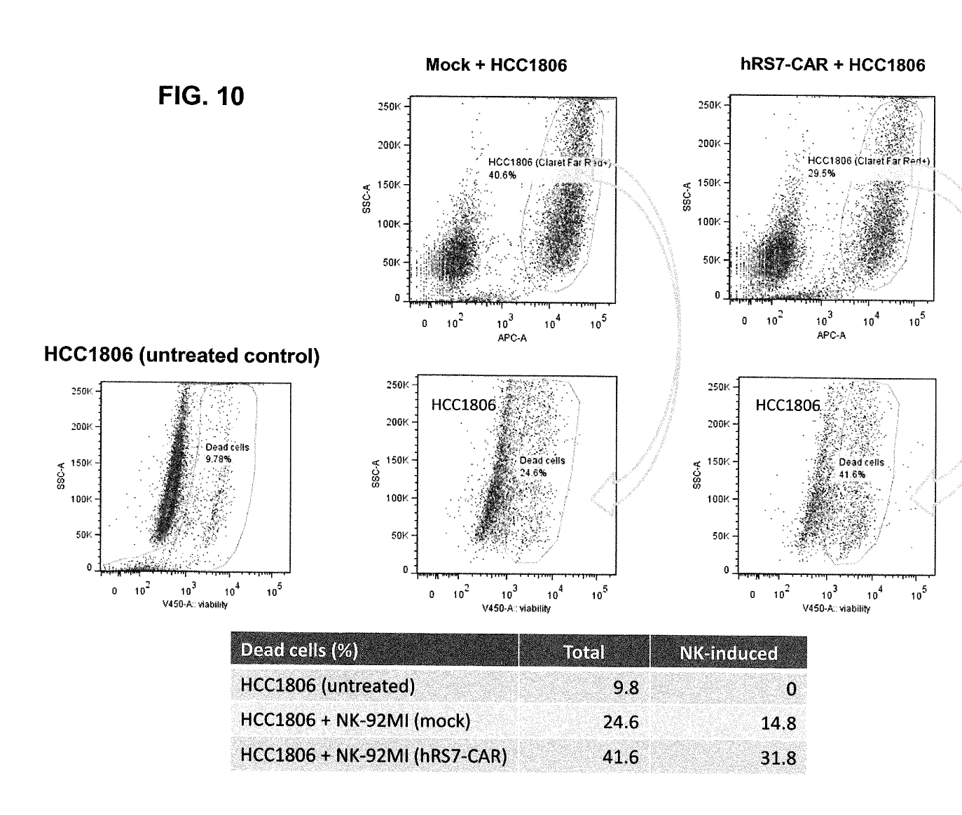

To further demonstrate the enhanced cytotoxicity of NK-92MI transfected with hRS7-CAR mRNA on targeted cells, HCC1806 cells were labeled with the CellVue Claret Far Red Fluorescent Cell Linker Kit (Sigma-Aldrich, Louis, Mo.) and incubated with NK-92MI cells at an effector to target ratio of 3:1 for 3 h at 37° C. Cells were then stained with BD V450 and analyzed by flow cytometry for viability of HCC1806. As shown in FIG. 10, about 42% of HCC1806 cells were killed by NK-92MI cells transfected with hRS7-CAR mRNA, in comparison to about 25% by mock-transfected NK-92 MI cells. Because about 10% of untreated HCC1806 cells were found not viable in the same experiment, the specific lysis of HCC1806 cells by NK-92MI cells transfected with hRS7-CAR mRNA was about 2-fold higher than that observed for mock-transfected NK-92MI.

### Example 27

### Generation of hRS7-CAR-NK-92ML Using Lentiviral Transduction

Lentiviral Packaging and Transduction

Lenti-X 293T cells were seeded overnight at 5×10⁶ cells/10-cm dish in 8 ml of growth medium, and reached 80-90% confluent at the time of transfection. A solution of the lentiviral vector, pLVX-puro-hRS7-CAR or pLVX-puro, was prepared in sterile water to contain 7 μg DNA in 600 μl, which was added to a tube of Lenti-X Packaging Single Shots (Clontech Laboratories). Samples were vortexed, incubated for 10 min at room temperature, centrifuged for 2 sec, and then added dropwise to the 8 ml of cell culture. After 4 h to overnight incubation at 37° C./5% CO₂, 6 ml of fresh complete growth medium was added and supernatants were harvested 48 h after the addition of viral vector.

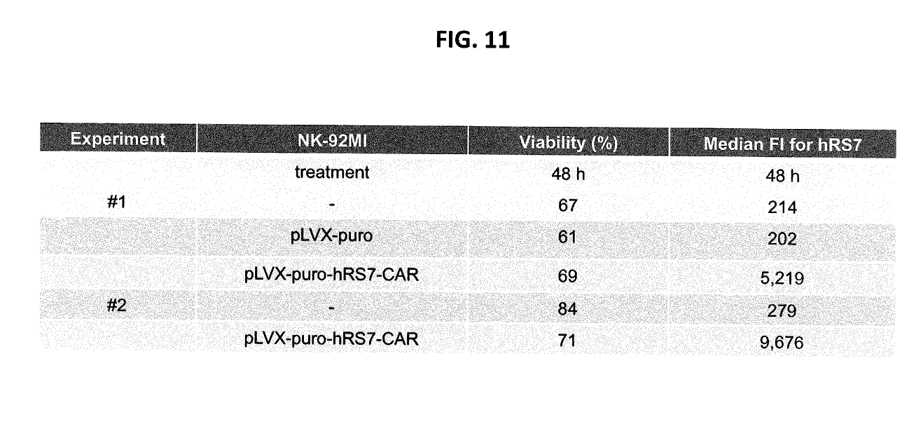

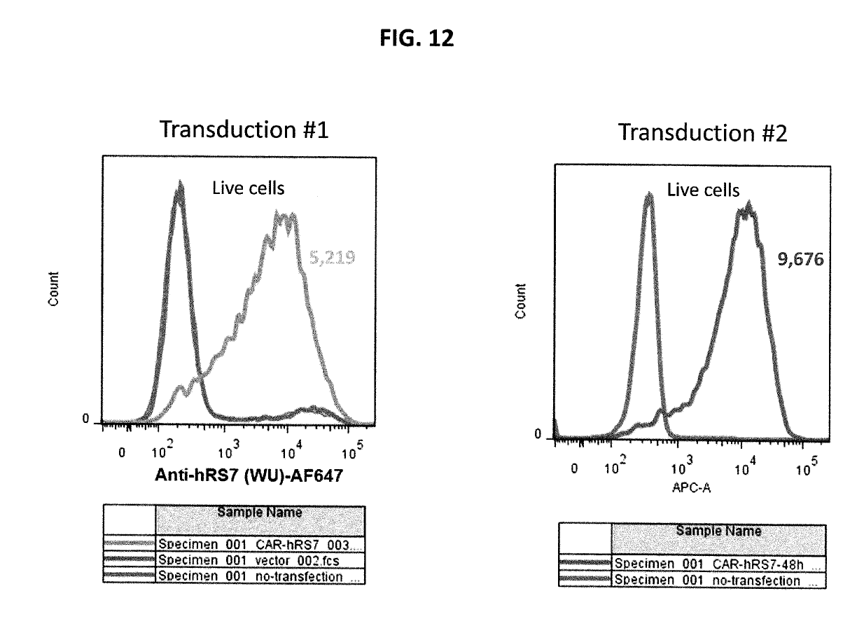

To transduce NK-92MI cells, harvested lentiviral supernatants were mixed with ¼ volumes of Lenti-X concentrator, incubated at 4° C. overnight, and added 1 ml of NK-92ML cells (2×10⁵) at log-phase on the next day. The cell-virus mixture was centrifuged for 15 min at 3,000 rpm, suspended in 1 ml of Myelocult medium supplemented with 4 g/ml retronectin (Clontech Laboratories) and incubated at 37° C./5% CO₂ for 24 h, followed by adding 1 ml of fresh Myelocult medium. After a further incubation for 24 h, spent media was discarded and replaced with 8 ml fresh media, from which portions of cells were removed, stained with BD V450 (BD Biosciences, San Jose, Calif.) and WU-AF-647 sequentially, washed with PBS plus 1% BSA, and assessed for viability and expression of hRS7 by flow cytometry. The results of two transductions are summarized in FIG. 11. The viability of NK-92MI cells transduced with pLVX-puro-hRS7-CAR was about 70% in both experiments, with similar viability observed for NK-92MI cells transduced with pLVX-puro (61%, experiment 1) and not transduced (67, experiment 1; 84%, experiment 2) (data not shown). The histograms presented in FIG. 12 show hRS7 was expressed (MFI>5,000) in the live population of NK-92MI cells transduced with pLVX-puro-hRS7-CAR, but not in the live population of NK-92MI cells transduced with pLVX-puro or not transduced.

All of the COMPOSITIONS and METHODS disclosed and claimed herein can be made and used without undue experimentation in light of the present disclosure. While the compositions and methods have been described in terms of preferred embodiments, it is apparent to those of skill in the art that variations maybe applied to the COMPOSITIONS and METHODS and in the steps or in the sequence of steps of the METHODS described herein without departing from the concept, spirit and scope of the invention. More specifically, certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of inducing an immune response to a disease comprising:
a) predosing a subject with an unconjugated antibody against a disease-associated antigen; and
b) administering to the subject a chimeric antigen receptor transfected T cell (CAR-T) or chimeric antigen receptor transfected NK cell (CAR-NK), wherein the chimeric antigen receptor (CAR) comprises a targeting antibody fragment against the same antigen.

2. The method of claim 1, wherein the unconjugated antibody and the targeting antibody fragment bind to the same epitope of the antigen.

3. The method of claim 1, wherein the same antibody is used for the targeting antibody fragment and the unconjugated antibody.

4. The method of claim 1, wherein the antigen is a B-cell antigen and the disease is selected from the group consisting of a hematopoietic cancer, an autoimmune disease and immune system dysfunction.

5. The method of claim 4, wherein the B-cell antigen is selected from the group consisting of CD19, CD20, CD21, CD22, CD44, CD62L, CD74, CD79b, HLA-DR, β7-integrin and BCR.

6. The method of claim 1, wherein the antigen is a tumor-associated antigen (TAA) and the disease is cancer.

7. The method of claim 6, wherein the TAA is selected from the group consisting of alpha-fetoprotein (AFP), α-actinin-4, A3, antigen specific for A33 antibody, ART-4, B7, Ba 733, BAGE, BrE3-antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CTLA4, CXCR4, CXCR7, CXCL12, HIF-1α, colon-specific antigen-p (CSAp), CEA (CEACAM-5), CEACAM-6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-β, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, PD1 receptor, placental growth factor, p53, PLAGL2, prostatic acid phosphatase, PSA, PRAME, PSMA, PlGF, ILGF, ILGF-R, L-6, IL-25, RS5, RANTES, T101, SAGE, S100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker and an oncogene product.

8. The method of claim 3, wherein the antibody is selected from the group consisting of hR1 (anti-IGF-1R), hPAM4 (anti-mucin), KC4 (anti-mucin), hA20 (anti-CD20), hA19 (anti-CD19), hIMMU31 (anti-AFP), hLL1 (anti-CD74), hLL2 (anti-CD22), RFB4 (anti-CD22), hMu-9 (anti-CSAp), hL243 (anti-HLA-DR), hMN-14 (anti-CEACAM-5), hMN-15 (anti-CEACAM-6), hRS7 (anti-TROP-2), hMN-3 (anti-CEACAM-6), CC49 (anti-TAG-72), J591 (anti-PSMA), D2/B (anti-PSMA), G250 (anti-carbonic anhydrase IX), infliximab (anti-TNF-α), certolizumab pegol (anti-TNF-α), adalimumab (anti-TNF-α), alemtuzumab (anti-CD52), bevacizumab (anti-VEGF), cetuximab (anti-EGFR), gemtuzumab (anti-CD33), ibritumomab tiuxetan (anti-CD20), panitumumab (anti-EGFR), rituximab (anti-CD20), tositumomab (anti-CD20), GA101 (anti-CD20), trastuzumab (anti-HER2/neu), tocilizumab (anti-IL-6 receptor), basiliximab (anti-CD25), daclizumab (anti-CD25), efalizumab (anti-CD11a), muromonab-CD3 (anti-CD3 receptor), natalizumab (anti-α4 integrin), BWA-3 (anti-histone H2A/H4), LG2-1 (anti-histone H3), MRA12 (anti-histone H1), PR1-1 (anti-histone H2B), LG11-2 (anti-histone H2B), and LG2-2 (anti-histone H2B).

9. The method of claim 1, wherein the CAR further comprises one or more elements selected from the group consisting of a leader peptide, a linker sequence, a transmembrane domain, an endodomain and a co-stimulatory domain.

10. The method of claim 9, wherein the leader peptide is a CD8α leader peptide.

11. The method of claim 9, wherein the leader peptide has an amino acid sequence of SEQ ID NO:18.

12. The method of claim 9, wherein the linker sequence comprises a CD8α hinge.

13. The method of claim 9, wherein the endodomain is selected from the group consisting of CD28 endodomain and CD3ζ endodomain.

14. The method of claim 9, wherein the co-stimulatory domain is selected from the group consisting of 4-1BB, OX40, Lck, DAP10 and ICOS.

15. The method of claim 1, further comprising administering to the subject at least one therapeutic agent selected from the group consisting of (i) an interferon; (ii) a checkpoint inhibitor antibody; (iii) an antibody-drug conjugate (ADC); (iv) an anti-HLA-DR antibody; and (v) an anti-CD74 antibody.

16. The method of claim 15, wherein the interferon is interferon-α.

17. The method of claim 15, wherein the checkpoint inhibitor antibody is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101 and tremelimumab.

18. The method of claim 15, wherein the antibody-drug conjugate is selected from the group consisting of hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine.

19. The method of claim 15, wherein the anti-CD74 antibody is hLL1 (milatuzumab) or the anti-HLA-DR antibody is hL243.

20. The method of claim 4, wherein the hematopoietic cancer is selected from the group consisting of B-cell leukemia, B-cell lymphoma, Hodgkin lymphoma, non-Hodgkin's lymphoma, Burkitt lymphoma, mantle cell lymphoma, acute lymphocytic leukemia, chronic lymphocytic leukemia, hairy cell leukemia, multiple myeloma and Waldenstrom's macroglobulinemia.

21. The method of claim 20, wherein the hematopoietic cancer is non-Hodgkin's lymphoma or chronic lymphocytic leukemia.

22. The method of claim 4, wherein the autoimmune disease is selected from the group consisting of acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, ANCA-associated vasculitides, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, bullous pemphigoid, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis and fibrosing alveolitis.

23. The method of claim 4, wherein the autoimmune disease is SLE (systemic lupus erythematosus).

24. The method of claim 4, wherein the immune dysfunction disease is selected from the group consisting of graft-versus-host disease, organ transplant rejection, septicemia, sepsis and inflammation.

25. The method of claim 6, wherein the cancer is selected from the group consisting of B-cell lymphoma, B-cell leukemia, colon cancer, stomach cancer, esophageal cancer, medullary thyroid cancer, kidney cancer, breast cancer, lung cancer, pancreatic cancer, urinary bladder cancer, ovarian cancer, uterine cancer, cervical cancer, testicular cancer, prostate cancer, liver cancer, skin cancer, bone cancer, brain cancer, rectal cancer, and melanoma.

26. The method of claim 1, further comprising administering to the subject a therapeutic agent selected from the group consisting of a second antibody or antigen-binding fragment thereof, a drug, a toxin, an enzyme, a cytotoxic agent, an anti-angiogenic agent, a pro-apoptotic agent, an antibiotic, a hormone, an immunomodulator, a cytokine, a chemokine, an antisense oligonucleotide, a small interfering RNA (siRNA), a boron compound and a radioisotope.

27. The method of claim 1, wherein predosing with unconjugated antibody reduces cytotoxicity to normal cells, but does not prevent an immune response against disease-associated cells.

28. The method of claim 1, wherein the predose is administered between 1 and 10 days prior to administration of CAR-T or CAR-NK.

29. The method of claim 28, wherein the predose is administered between 3 to 7 days prior to administration of CAR-T or CAR-NK.

30. The method of claim 26, wherein the predose is administered between 4 to 6 days prior to administration of CAR-T or CAR-NK.

31. The method of claim 28, wherein the administration of predose is repeated, after a delay of up to 7 days.

32. The method of claim 1, wherein the unconjugated antibody is a chimeric, humanized or human antibody.

33. The method of claim 1, wherein the unconjugated antibody is has IgG1, IgG2 or IgG4 constant region sequences.

34. The method of claim 1, wherein the unconjugated antibody has IgG4 constant regions and a Ser241Pro hinge mutation.

35. The method of claim 1, wherein the antibody fragment is a scFv or Fab antibody fragment.

36. The method of claim 1, wherein the CAR-T comprises a transfected CD8+ T cell and/or a CD8+ memory T cell.

37. The method of claim 1, wherein the CAR-NK comprises an NK cell selected from the group consisting of primary NK cells, NK-92, NK-92.26.5, NK 92.MI, NK-92Ci, NK-92Fc, NK3.3, NKL, NKG, NK-YT, NK-YTS, KHYG-1 and HATAK cells.

38. A method of inducing an immune response to a disease comprising:
a) predosing a subject with an unconjugated antibody against a disease-associated antigen;
b) administering to the subject a hapten-conjugated antibody against the same antigen; and
c) administering to the subject a CAR-T or CAR-NK, wherein the chimeric antigen receptor (CAR) comprises an anti-hapten antibody fragment.

39. The method of claim 38, wherein the hapten is HSG or In-DTPA.

40. The method of claim 39, wherein the anti-hapten antibody is h679 or h734.

41. The method of claim 38, wherein the unconjugated antibody and the hapten-conjugated antibody fragment bind to the same epitope of the disease-associated antigen.

42. The method of claim 38, wherein the same antibody is used for the hapten-conjugated antibody fragment and the unconjugated antibody.

43. The method of claim 38, wherein the antigen is a B-cell antigen and the disease is selected from the group consisting of a hematopoietic cancer, an autoimmune disease and immune system dysfunction.

44. The method of claim 43, wherein the B-cell antigen is selected from the group consisting of CD19, CD20, CD21, CD22, CD44, CD62L, CD74, CD79b, HLA-DR, β7-integrin and BCR.

45. The method of claim 38, wherein the antigen is a tumor-associated antigen (TAA) and the disease is cancer.

46. The method of claim 45, wherein the TAA is selected from the group consisting of alpha-fetoprotein (AFP), α-actinin-4, A3, antigen specific for A33 antibody, ART-4, B7, Ba 733, BAGE, BrE3-antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CTLA4, CXCR4, CXCR7, CXCL12, HIF-1α, colon-specific antigen-p (CSAp), CEA (CEACAM-5), CEACAM-6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-β, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, PD1 receptor, placental growth factor, p53, PLAGL2, prostatic acid phosphatase, PSA, PRAME, PSMA, PlGF, ILGF, ILGF-R, L-6, IL-25, RS5, RANTES, T101, SAGE, S100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker and an oncogene product.

47. The method of claim 42, wherein the antibody is selected from the group consisting of hR1 (anti-IGF-1R), hPAM4 (anti-mucin), KC4 (anti-mucin), hA20 (anti-CD20), hA19 (anti-CD19), hIMMU31 (anti-AFP), hLL1 (anti-CD74), hLL2 (anti-CD22), RFB4 (anti-CD22), hMu-9 (anti-CSAp), hL243 (anti-HLA-DR), hMN-14 (anti-CEACAM-5), hMN-15 (anti-CEACAM-6), hRS7 (anti-TROP-2), hMN-3 (anti-CEACAM-6), CC49 (anti-TAG-72), J591 (anti-PSMA), D2/B (anti-PSMA), G250 (anti-carbonic anhydrase IX), infliximab (anti-TNF-α), certolizumab pegol (anti-TNF-α), adalimumab (anti-TNF-α), alemtuzumab (anti-CD52), bevacizumab (anti-VEGF), cetuximab (anti-EGFR), gemtuzumab (anti-CD33), ibritumomab tiuxetan (anti-CD20), panitumumab (anti-EGFR), rituximab (anti-CD20), tositumomab (anti-CD20), GA101 (anti-CD20), trastuzumab (anti-HER2/neu), tocilizumab (anti-IL-6 receptor), basiliximab (anti-CD25), daclizumab (anti-CD25), efalizumab (anti-CD11a), muromonab-CD3 (anti-CD3 receptor), natalizumab (anti-α4 integrin), BWA-3 (anti-histone H2A/H4), LG2-1 (anti-histone H3), MRA12 (anti-histone H1), PR1-1 (anti-histone H2B), LG11-2 (anti-histone H2B), and LG2-2 (anti-histone H2B).

48. The method of claim 38, further comprising administering to the subject at least one therapeutic agent selected from the group consisting of (i) an interferon; (ii) a checkpoint inhibitor antibody; and (iii) an antibody-drug conjugate (ADC).

49. The method of claim 38, further comprising administering to the subject a therapeutic agent selected from the group consisting of a drug, a toxin, an enzyme, a cytotoxic agent, an anti-angiogenic agent, a pro-apoptotic agent, an antibiotic, a hormone, an immunomodulator, a cytokine, a chemokine, an antisense oligonucleotide, a small interfering RNA (siRNA), a boron compound and a radioisotope.

50. The method of claim 38, wherein predosing with unconjugated antibody reduces cytotoxicity to normal cells, but does not prevent an immune response against disease-associated cells.

51. The method of claim 38, wherein the predose is administered between 1 and 10 days prior to administration of CAR-T or CAR-NK.

52. The method of claim 51, wherein the predose is administered between 3 to 7 days prior to administration of CAR-T or CAR-NK.

53. The method of claim 51, wherein the predose is administered between 4 to 6 days prior to administration of CAR-T or CAR-NK.

54. The method of claim 38, wherein the administration of predose is repeated, after a delay of up to 7 days.

55. The method of claim 38, wherein the unconjugated antibody is a chimeric, humanized or human antibody.

56. The method of claim 38, wherein the unconjugated antibody is has IgG1, IgG2 or IgG4 constant region sequences.

57. The method of claim 38, wherein the unconjugated antibody has IgG4 constant regions and a Ser241Pro hinge mutation.

58. A CAR construct comprising an anti-hapten antibody fragment.

59. A T-cell (CAR-T) or NK cell (CAR-NK) transduced with a CAR construct according to claim 58.

60. A pharmaceutical composition comprising a CAR-T or CAR-NK according to claim 56.

61. The method of claim 38, wherein the CAR further comprises one or more elements selected from the group consisting of a leader peptide, a linker sequence, a transmembrane domain, an endodomain and a co-stimulatory domain.

62. The method of claim 61, wherein the leader peptide is a CD8α leader peptide, the linker sequence comprises a CD8α hinge, the endodomain is selected from the group consisting of CD28 endodomain and CD3ζ endodomain, and/or the co-stimulatory domain is selected from the group consisting of 4-1BB, OX40, Lck, DAP10 and ICOS.

63. A method of inducing an immune response to a Trop-2 expressing cancer comprising administering to a subject with a Trop-2 expressing cancer a CAR-T or CAR-NK, wherein the chimeric antigen receptor (CAR) comprises an anti-Trop-2 antibody fragment.

64. The method of claim 63, further comprising administering to the subject at least one therapeutic agent selected from the group consisting of (i) an interferon; (ii) a checkpoint inhibitor antibody; (iii) an antibody-drug conjugate (ADC); (iv) an anti-HLA-DR antibody; and (v) an anti-CD74 antibody.

65. The method of claim 64, wherein the interferon is interferon-α, the checkpoint inhibitor antibody is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101 and tremelimumab, and/or the antibody-drug conjugate is selected from the group consisting of hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine.

66. The method of claim 64, wherein the anti-CD74 antibody is hLL1 (milatuzumab) or the anti-HLA-DR antibody is hL243.

67. The method of claim 63, wherein the Trop-2 expressing cancer is a carcinoma of the esophagus, pancreas, lung, stomach, colon, rectum, urinary bladder, breast, ovary, uterus, kidney or prostate.

68. The method of claim 63, further comprising administering to the subject a therapeutic agent selected from the group consisting of a second antibody or antigen-binding fragment thereof, a drug, a toxin, an enzyme, a cytotoxic agent, an anti-angiogenic agent, a pro-apoptotic agent, an antibiotic, a hormone, an immunomodulator, a cytokine, a chemokine, an antisense oligonucleotide, a small interfering RNA (siRNA), a boron compound and a radioisotope.

69. A CAR construct comprising an anti-Trop-2 antibody fragment.

70. The CAR construct of claim 69, wherein the CAR further comprises one or more elements selected from the group consisting of a leader peptide, a linker sequence, a transmembrane domain, an endodomain and a co-stimulatory domain.

71. A T-cell (CAR-T) or NK cell (CAR-NK) transduced with a CAR construct according to claim 70.

72. A pharmaceutical composition comprising a CAR-T or CAR-NK according to claim 70.

73. A method of inducing an immune response to a tumor-associated antigen (TAA) expressing cancer, comprising
a) administering to a subject with cancer a hapten-conjugated anti-TAA antibody; and
b) administering to the subject a CAR-T or CAR-NK, wherein the chimeric antigen receptor (CAR) comprises an anti-hapten antibody fragment.

74. The method of claim 73, wherein the hapten is HSG or In-DTPA.

75. The method of claim 74, wherein the anti-hapten antibody is h679 or h734.

76. The method of claim 73, wherein the TAA is selected from the group consisting of carbonic anhydrase IX, alpha-fetoprotein, α-actinin-4, A3, antigen specific for A33 antibody, ART-4, B7, Ba 733, BAGE, BrE3-antigen, CA125, CAMEL, CAP-1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CXCR4, CXCR7, CXCL12, HIF-1α, colon-specific antigen-p (CSAp), CEA (CEACAM-5), CEACAM-6, c-met, DAM, EGFR, EGFRvIII, EGP-1, EGP-2, ELF2-M, Ep-CAM, Flt-1, Flt-3, folate receptor, G250 antigen, GAGE, gp100, GROB, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, placental growth factor, p53, PLAGL2, prostatic acid phosphatase, PSA, PRAME, PSMA, PlGF, ILGF, ILGF-R, IL-6, IL-25, RS5, RANTES, T101, SAGE, S100, survivin, survivin-2B, TAC, TAG-72, tenascin, TRAIL receptors, TNF-α, Tn antigen, Thomson-Friedenreich antigens, tumor necrosis antigens, TROP-2, VEGFR, ED-B fibronectin, WT-1, 17-1A-antigen, complement factors C3, C3a, C3b, C5a, C5, an angiogenesis marker, bcl-2, bcl-6, Kras, cMET, and an oncogene product.

77. The method of claim 76, wherein the anti-TAA antibody is selected from the group consisting of hR1 (anti-IGF-1R), hPAM4 (anti-mucin), KC4 (anti-mucin), hA20 (anti-CD20), hA19 (anti-CD19), hIMMU31 (anti-AFP), hLL1 (anti-CD74), hLL2 (anti-CD22), RFB4 (anti-CD22), hMu-9 (anti-CSAp), hL243 (anti-HLA-DR), hMN-14 (anti-CEACAM-5), hMN-15 (anti-CEACAM-6), hRS7 (anti-TROP-2), hMN-3 (anti-CEACAM-6), CC49 (anti-TAG-72), J591 (anti-PSMA), D2/B (anti-PSMA), G250 (anti-carbonic anhydrase IX), infliximab (anti-TNF-α), certolizumab pegol (anti-TNF-α), adalimumab (anti-TNF-α), alemtuzumab (anti-CD52), bevacizumab (anti-VEGF), cetuximab (anti-EGFR), gemtuzumab (anti-CD33), ibritumomab tiuxetan (anti-CD20), panitumumab (anti-EGFR), rituximab (anti-CD20), tositumomab (anti-CD20), GA101 (anti-CD20), trastuzumab (anti-HER2/neu), tocilizumab (anti-IL-6 receptor), basiliximab (anti-CD25), daclizumab (anti-CD25), efalizumab (anti-CD11a), muromonab-CD3 (anti-CD3 receptor), natalizumab (anti-α4 integrin), BWA-3 (anti-histone H2A/H4), LG2-1 (anti-histone H3), MRA12 (anti-histone H1), PR1-1 (anti-histone H2B), LG11-2 (anti-histone H2B), and LG2-2 (anti-histone H2B).

78. The method of claim 73, further comprising administering to the subject at least one therapeutic agent selected from the group consisting of (i) an interferon; (ii) a checkpoint inhibitor antibody; (iii) an antibody-drug conjugate (ADC); (iv) an anti-HLA-DR antibody; and (v) an anti-CD74 antibody.

79. The method of claim 73, further comprising administering to the subject a therapeutic agent selected from the group consisting of a drug, a toxin, an enzyme, a cytotoxic agent, an anti-angiogenic agent, a pro-apoptotic agent, an antibiotic, a hormone, an immunomodulator, a cytokine, a chemokine, an antisense oligonucleotide, a small interfering RNA (siRNA), a boron compound and a radioisotope.


SEQUENCE LISTING (29 sequences)

SEQ ID NO: 1 - protein, 21 aa, Artificial Sequence
  Description of Artificial Sequence Synthetic peptide
MALPVTALLLPLALLLHAARP

SEQ ID NO: 2 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 3 - protein, 24 aa, Artificial Sequence
  Description of Artificial Sequence Synthetic peptide
IYIWAPLAGTCGVLLLSLVITLYC

SEQ ID NO: 4 - protein, 27 aa, Artificial Sequence
  Description of Artificial Sequence Synthetic peptide
FWVLVVVGGVLACYSLLVTVAFIIFWV

SEQ ID NO: 5 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 43 aa, Artificial Sequence
  Description of Artificial Sequence Synthetic polypeptide
RSKRSRLLHSDYMNMTPRRPGPTRKHYQPYAPPRDFAAYRSID

SEQ ID NO: 7 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 8 - protein, 117 aa, Artificial Sequence
  Description of Artificial Sequence Synthetic polypeptide
QVQLQESGGGLVQPGGSLRLSCAASGFTFSHYTMSWVRQAPGKGLEWVTYITNGGVSSYHPDTVKGRFTVSRDNSKNTLYLQMNSLRAEDTAVYFCTRHAVYAFAYWGQGSLVTVSS

SEQ ID NO: 9 - protein, 112 aa, Artificial Sequence
  Description of Artificial Sequence Synthetic polypeptide
DIQLVVTQEPSFSVSPGGTVTFTCRSSAGAVTTSNYANWVQEKPGQAPRGLIGGTTNRAPGVPARFSGSILGNKAALTITGAQADDESIYFCVLWYSDRWVFGGGTKLKIKR

SEQ ID NO: 10 - protein, 119 aa, Artificial Sequence
  Description of Artificial Sequence Synthetic polypeptide
QVQLQESGGDLVKPGGSLKLSCAASGFTFSIYTMSWLRQTPGKGLEWVATLSGDGDDIYYPDSVKGRFTISRDNAKNSLYLQMNSLRAEDTALYYCARVRLGDWDFDVWGQGTTVTVSS

SEQ ID NO: 11 - protein, 113 aa, Artificial Sequence
  Description of Artificial Sequence Synthetic polypeptide
DIQLTQSPSSLAVSPGERVTLTCKSSQSLFNSRTRKNYLGWYQQKPGQSPKLLIYWASTRESGVPDRFSGSGSGTDFTLTINSLQAEDVAVYYCTQVYYLCTFGAGTKLEIKR

SEQ ID NO: 12 - protein, 121 aa, Artificial Sequence
  Description of Artificial Sequence Synthetic polypeptide
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 13 - protein, 104 aa, Artificial Sequence
  Description of Artificial Sequence Synthetic polypeptide
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKV

SEQ ID NO: 14 - protein, 121 aa, Artificial Sequence
  Description of Artificial Sequence Synthetic polypeptide
QVQLQESGGGVVQPGRSLRLSCSSSGFALTDYYMSWVRQAPGKGLEWLGFIANKANGHTTDYSPSVKGRFTISRDNSKNTLFLQMDSLRPEDTGVYFCARDMGIRWNFDVWGQGTPVTVSS

SEQ ID NO: 15 - protein, 107 aa, Artificial Sequence
  Description of Artificial Sequence Synthetic polypeptide
DIQLTQSPSSLSASVGDRVTMTCSASSRVSYIHWYQQKPGKAPKRWIYGTSTLASGVPARFSGSGSGTDFTFTISSLQPEDIATYYCQQWSYNPPTFGQGTKVEIKR

SEQ ID NO: 16 - protein, 119 aa, Artificial Sequence
  Description of Artificial Sequence Synthetic polypeptide
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 17 - protein, 107 aa, Artificial Sequence
  Description of Artificial Sequence Synthetic polypeptide
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKR

SEQ ID NO: 18 - protein, 15 aa, Artificial Sequence
  Description of Artificial Sequence Synthetic peptide
GGGGSGGGGSGGGGS

SEQ ID NO: 19 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 491 aa, Artificial Sequence
  Description of Artificial Sequence Synthetic polypeptide
MALPVTALLLPLALLLHAARPDIQLTQSPSSLAVSPGERVTLTCKSSQSLFNSRTRKNYLGWYQQKPGQSPKLLIYWASTRESGVPDRFSGSGSGTDFTLTINSLQAEDVAVYYCTQVYYLCTFGAGTKLEIKRGGGGSGGGGSGGGGSQVQLQESGGDLVKPGGSLKLSCAASGFTFSIYTMSWLRQTPGKGLEWVATLSGDGDDIYYPDSVKGRFTISRDNAKNSLYLQMNSLRAEDTALYYCARVRLGDWDFDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 537 aa, Artificial Sequence
  Description of Artificial Sequence Synthetic polypeptide
MALPVTALLLPLALLLHAARPDIQLTQSPSSLAVSPGERVTLTCKSSQSLFNSRTRKNYLGWYQQKPGQSPKLLIYWASTRESGVPDRFSGSGSGTDFTLTINSLQAEDVAVYYCTQVYYLCTFGAGTKLEIKRGGGGSGGGGSGGGGSQVQLQESGGDLVKPGGSLKLSCAASGFTFSIYTMSWLRQTPGKGLEWVATLSGDGDDIYYPDSVKGRFTISRDNAKNSLYLQMNSLRAEDTALYYCARVRLGDWDFDVWGQGTTVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 4 aa, Artificial Sequence
  Description of Artificial Sequence Synthetic peptide
AKYK

SEQ ID NO: 24 - DNA, 26 bp, Artificial Sequence
  Description of Artificial Sequence Synthetic primer
tcaactcgagcgccgccaccatggcc

SEQ ID NO: 25 - DNA, 29 bp, Artificial Sequence
  Description of Artificial Sequence Synthetic primer
ctggtctagaggtaaccctaccgtggtgg

SEQ ID NO: 26 - protein, 487 aa, Artificial Sequence
  Description of Artificial Sequence Synthetic polypeptide
MALPVTALLLPLALLLHAARPDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1695 bp, Artificial Sequence
  Description of Artificial Sequence Synthetic polynucleotide
tctagataatacgactcactatagggagagcttgttctttttgcagaagctcagaataaacgctcaactttggcgccgccaccatggccctgcccgtgaccgccctgctgctgcccctggccctgctgctgcacgccgcaagacccgacattcagctgacccagtctccatcctccctgtctgcatctgtaggagacagagtcagcatcacctgcaaggccagtcaggatgtgagtattgctgtagcctggtatcagcagaaaccagggaaagcccctaagctcctgatctactcggcatcctaccggtacactggagtccctgataggttcagtggcagtggatctgggacagatttcactctcaccatcagcagtctgcaacctgaagattttgcagtttattactgtcagcaacattatattactccgctcacgttcggtgctgggaccaaggtggagatcaaaggtggaggagggtccggtggaggagggtctggtggaggagggagccaggtccagctgcagcaatctgggtctgagttgaagaagcctggggcctcagtgaaggtttcctgcaaggcttctggatacaccttcacaaactatggaatgaactgggtgaagcaggcccctggacaagggcttaaatggatgggctggataaacacctacactggagagccaacatatactgatgacttcaagggacggtttgccttctccttggacacctctgtcagcacggcatatctccagatcagcagcctaaaggctgacgacactgccgtgtatttctgtgcaagaggggggttcggtagtagctactggtacttcgatgtctggggccaagggtccctggtcaccgtctcctcaaccacaaccccagcaccaagaccacctacacctgcaccaaccatcgccagccagcctctgtccctgagaccagaggcatgtaggccagcagcaggaggagcagtgcacaccaggggcctggatttcgcctgcgacatctacatctgggcaccactggcaggaacatgtggcgtgctgctgctgtctctggtcatcaccctgtactgcaagagaggcaggaagaagctgctgtatatcttcaagcagcccttcatgcgccccgtgcagacaacccaggaggaggatggctgctcctgtcggttcccagaagaagaggagggaggatgtgagctgagggtgaagtttagccggtccgccgacgcaccagcataccagcagggccagaaccagctgtataacgagctgaatctgggccggagagaggagtacgatgtgctggacaagaggcgcggcagagatcctgagatgggcggcaagcctcggagaaagaacccacaggagggcctgtacaatgagctgcagaaggataagatggccgaggcctatagcgagatcggcatgaagggagagaggcgccggggcaagggacacgacggcctgtatcagggcctgtccaccgcaaccaaggatacctatgacgcactgcacatgcaggctctgccaccacggtagggttaccactaaaccagcctcaagaacacccgaatggagtctctaagctacataataccaacttacactttacaaaatgttgtcccccaaaatgtagccattcgtatctgctcctaataaaaagaaagtttcttcacattctaagctt

SEQ ID NO: 28 - protein, 107 aa, Artificial Sequence
  Description of Artificial Sequence Synthetic polypeptide
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 29 - protein, 41 aa, Artificial Sequence
  Description of Artificial Sequence Synthetic polypeptide
TTTPAPRPPTPAPTIASQPLSLRPEACRPAAGGAVHTRGLD